(19) United States
(12) Patent Application Publication
(10) Pub. No.: US 2025/0313577 A1
(43) Pub. Date: Oct. 9, 2025

(54) TRICYCLIC PYRIDONES AND PYRIMIDONES

(71) Applicant: ERASCA, INC, San Diego, CA (US)

(72) Inventors: Marcos Gonzalez-Lopez, San Diego, CA (US); Jun Feng, San Diego, CA (US); Jean-Michel Vernier, San Diego, CA (US); Nicholas A. Isley, San Diego, CA (US); Ping Chen, San Diego, CA (US)

(21) Appl. No.: 18/860,263

(22) PCT Filed: Apr. 25, 2023
(86) PCT No.: PCT/US2023/066170

§ 371 Date: Oct. 25, 2024

Related U.S. Application Data
(60) Provisional application No. 63/363,624, filed on Apr. 26, 2022.
(60) Provisional application No. 63/364,027, filed on May 2, 2022.

(51) Int. Cl. C07D 513/06; A61K 31/554

(52) U.S. Cl. CPC C07D 513/06; A61K 31/554

(57) ABSTRACT

Compounds with KRAS G12C inhibitory active are disclosed and methods of using the same to treat a cancer comprising a K-Ras G12C mutation.

### REFERENCE TO SEQUENCE LISTING

This application incorporates by reference a Computer Readable Form (CRF) of a Sequence Listing in ASCII text format submitted with this application, entitled 2023-04-24 Sequence_Listing_ST26 055745-542001WO.xml, was created on Apr. 24, 2023, and is 5,290 bytes in size.

### BACKGROUND

Embodiments herein relate to compounds and methods for the treatment of RAS-mediated disease. In particular, embodiments herein relate to compounds and methods for treating diseases such as cancer via targeting oncogenic mutants of the K-RAS isoform.

Ras proteins are small guanine nucleotide-binding proteins that act as molecular switches by cycling between active GTP-bound and inactive GDP-bound conformations. Ras signaling is regulated through a balance between activation by guanine nucleotide exchange factors (GEFs), most commonly son of sevenless (SOS), and inactivation by GTPase-activating proteins (GAPs) such as neurofibromin or p120GAP. The Ras proteins play an important role in the regulation of cell proliferation, differentiation, and survival. Dysregulation of the Ras signaling pathway is almost invariably associated with disease. Hyper-activating somatic mutations in Ras are among the most common lesions found in human cancer. Most of these mutations have been shown to decrease the sensitivity of Ras to GAP stimulation and decrease its intrinsic GTPase activity, leading to an increase in the active GTP-bound population. Although mutation of any one of the three Ras isoforms (K-Ras, N-Ras, or H-Ras) has been shown to lead to oncogenic transformation, K-Ras mutations are by far the most common in human cancer. For example, K-Ras mutations are known to be often associated with pancreatic, colorectal and non-small-cell lung carcinomas. Similarly, H-Ras mutations are common in cancers such as papillary thyroid cancer, lung cancers and skin cancers. Finally, N-Ras mutations occur frequently in hepatocellular carcinoma.

There is a need for effective Ras inhibitors, which may provide a new class of anticancer compounds. These and other advantages will be apparent to those skilled in the art based upon embodiments and disclosures herein.

### SUMMARY

In some aspects, embodiments disclosed herein relate to compounds of Formula (I)

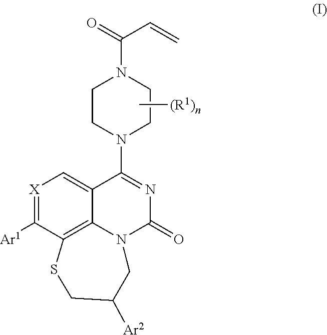

wherein:
   each R¹ is independently methyl or cyanomethyl;
   n is an integer from 0 to 2;
   X is —CCF₃ or —C-halogen;
   Ar¹ is aryl or heteroaryl, optionally substituted with one or more alkyl, halogen, hydroxy or amino; and
      Ar² is a C-linked aryl or heteroaryl, optionally substituted with alkyl or halogen.

In some aspects, embodiments herein relate to methods of treating a subject with cancer associated with a G12C Kras mutation comprising administering to the subject a compound, as disclosed herein, in a pharmaceutically acceptable vehicle.

### DETAILED DESCRIPTION

### I. General

Disclosed herein are potent and selective tricyclic quinazoline-2-ones compounds, which have been found to be useful as inhibitors of oncogenic mutants of RAS proteins. Among various advantages, the compounds disclosed herein are selective for oncogenic RAS mutants over wild-type RAS proteins. Further, compounds disclosed herein may exhibit selectivity for oncogenic mutants of K-RAS over other mutated K-RAS proteins, as well as mutants of the N-RAS and H-RAS isoforms. In particular, the compounds disclosed herein may exhibit selectivity for K-RAS, N-RAS, and H-RAS mutants having a common G12C mutation. Also disclosed herein are pharmaceutical compositions comprising these compounds, and their application in the treatment of disease, such as cancer. Methods of inhibition of oncogenic mutant K-RAS, N-RAS, and H-RAS activity are also provided, as well as methods for the treatment of oncogenic mutant RAS-mediated diseases, especially those involving elevated levels of oncogenic mutated RAS, in particular cancer.

Disclosed herein is a class of compounds useful in treating oncogenic RAS-mediated disorders and conditions, defined by structural Formula (I):

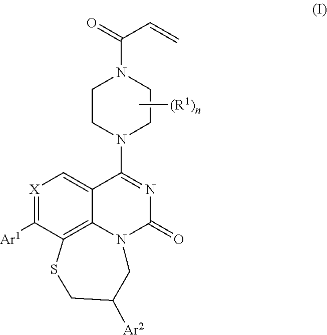

wherein:
   each R¹ is independently methyl or cyanomethyl;
   n is an integer from 0 to 2;
   X is —CCF₃ or —C-halogen;
   Ar¹ is aryl or heteroaryl, optionally substituted with one or more alkyl, halogen, hydroxy or amino; and
      Ar² is a C-linked aryl or heteroaryl, optionally substituted with alkyl or halogen.

Compounds according to the various embodiments disclosed herein possess useful oncogenic mutant RAS inhibiting or modulating activity, and may be used in the treatment or prophylaxis of a disease or condition in which oncogenic mutant RAS plays an active role. Thus, in a broad aspect, embodiments disclosed herein also provide pharmaceutical compositions comprising one or more compounds disclosed herein together with a pharmaceutically acceptable carrier, as well as methods of making and using the compounds and compositions. Embodiments disclosed herein provide methods for selectively inhibiting the RAS that are oncogenic mutants having the G12C mutation. In some embodiments, there are provided methods for treating an oncogenic mutant K-RAS-mediated disorder in a subject, comprising administering to the subject a therapeutically effective amount of a compound or pharmaceutical composition according to the various embodiments disclosed herein. Related embodiments disclose the use of the compounds disclosed herein as therapeutic agents, for example, in treating cancer and other diseases involving elevated levels of oncogenic mutant K-RAS. The various embodiments disclosed herein also contemplate the use of the compounds disclosed herein for use in the manufacture of a medicament for the treatment of a disease or condition ameliorated by the inhibition of oncogenic mutant K-RAS. In some such embodiments, the disease or condition is cancer. Each of the aforementioned methods apply equally to the similar mutation in N-RAS and H-RAS bearing the G12C mutation.

Compounds of the various embodiments disclosed herein may be selective amongst the RAS oncogenic mutant forms in various ways. For example, compounds described herein may be selective for G12C mutants of K-RAS, N-RAS, or H-RAS. In certain embodiments, compounds of the various embodiments disclosed herein may be selective for K-RAS G12C over other K-RAS mutants and Wild Type K-RAS. Likewise, compounds of various embodiments disclosed herein may be selective for N-RAS and H-RAS bearing the same G12C mutation.

The various embodiments disclosed herein also relate to methods of inhibiting at least one RAS function comprising the step of contacting an oncogenic mutant RAS with a compound of Formula I, as described herein. The cell phenotype, cell proliferation, activity of the mutant RAS, change in biochemical output produced by active mutant RAS, expression of mutant RAS, or binding of mutant RAS with a natural binding partner may be affected. Such methods may be embrace modes of treatment of disease, biological assays, cellular assays, biochemical assays, or the like.

### II. Definitions

### A. General Definitions

As used herein, the terms below have the meanings indicated.

When ranges of number values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure, taking into account significant figures.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

### B. Chemical Definitions

The following chemical functional group definitions are provided to give guidance in understanding their meaning and scope. Those skilled in the art will recognize that these functional groups are being used in a manner consistent with practice of the chemical arts. Any of the following chemical functional groups may be optionally substituted as defined below and each chemical functional group below may itself be an optional substitution.

The term “acyl,” as used herein, alone or in combination, refers to a carbonyl (C═O) attached to an alkenyl, alkyl, aryl, cycloalkyl, heteroaryl, heterocycloalkyl, or any other moiety were the atom attached to the carbonyl is carbon. An “acetyl” group, which is a type of acyl, refers to a (—C(═O)CH₃) group. An “alkylcarbonyl” or “alkanoyl” group refers to an alkyl group attached to the parent molecular moiety through a carbonyl group. Examples of such groups include, without limitation, methylcarbonyl and ethylcarbonyl. Similarly, an “arylcarbonyl” or “aroyl” group refers to an aryl group attached to the parent molecular moiety through a carbonyl group. Examples of such groups include, without limitation, benzoyl and naphthoyl. Accordingly, generic examples of acyl groups include alkanoyl, aroyl, heteroaroyl, and so on. Specific examples of acyl groups include, without limitation, formyl, acetyl, acryloyl, benzoyl, trifluoroacetyl and the like.

The term “alkenyl,” as used herein, alone or in combination, refers to a straight-chain or branched-chain hydrocarbon radical having one or more double bonds and containing from 2 to 20 carbon atoms. In certain embodiments, the alkenyl may comprise from 2 to 6 carbon atoms, or from 2 to 4 carbons, either of which may be referred to as “lower alkenyl.” The term “alkenylene” refers to a carbon-carbon double bond system attached at two or more positions such as ethenylene (—CH═CH—). Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆, and so on up to 20 carbon atoms. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted. Unless otherwise specified, the term “alkenyl” may include “alkenylene” groups.

The term “alkoxy,” as used herein, alone or in combination, refers to an alkyl ether radical, wherein the term alkyl is as defined below. Alkoxy groups may have the general formula: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, and the like. The alkoxy groups can be further optionally substituted as defined herein.

The term “alkyl,” as used herein, alone or in combination, (sometimes abbreviated Alk) refers to a straight-chain or branched-chain alkyl radical containing from 1 to 20 carbon atoms. In certain embodiments, the alkyl may comprise from 1 to 10 carbon atoms. In further embodiments, the alkyl may comprise from 1 to 6 carbon atoms, or from 1 to 4 carbon atoms. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups. When the alkyl is methyl, it may be represented structurally as CH₃, Me, or just a single bond terminating with no end group substitution.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino (—NHMe), N-ethylamino (—NHEt), N,N-dimethylamino (—NMe₂), N,N-ethylmethylamino (—NMeEt) and the like. The term “aminoalkyl” refers to reverse orientation in which the amino group appears distal to the parent molecular moiety and attachment to the parent molecular moiety is through the alkyl group. For example, NH₂(CH₂)ₙ-describes an aminoalkyl group with a terminal amine at the end of an alkyl group attached to the parent molecular moiety. The two terms alkylamino and aminoalkyl can be combined to describe an “alkylaminoalkyl” group in which an alkyl group resides on a nitrogen atom distal to the parent molecular moiety, such as MeNH(CH₂)ₙ—. In a similar manner, an aryl group, as defined herein, may combine in a similar fashion providing an arylaminoalkyl group ArNH(CH₂)ₙ—. For additional clarity nomenclature may be provided where the group that is attached to nitrogen is indicated so by use of “N-” in the name, such as N-arylaminoalkyl, which is understood to mean that the aryl group is a substituent on the nitrogen atom of the aminoalkyl group, the alkyl being attached the parent molecular moiety.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (AlkS-) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like. Similarly, “arylthio” refers to arylthioether (ArS-) radical wherein the term aryl is as defined herein and wherein the sulfur may be singly or double oxidized.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted. Unless otherwise specified, the term “alkynyl” may include “alkynylene” groups.

The terms “amido,” as used herein, alone or in combination, refer to an amino group as described below attached to the parent molecular moiety through a carbonyl group. The term “C-amido” as used herein, alone or in combination, refers to a —C(═O)N(R)₂ group where is R as defined herein. The term “N-amido” as used herein, alone or in combination, refers to RC(═O)N(R′)— group, with R and R′ as defined herein.

The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —N(R)(R′) or —N⁺(R)(R′)(R″), wherein R, R′ and R″ are independently selected from the group consisting of hydrogen, alkyl, acyl, heteroalkyl, aryl, cycloalkyl, heteroaryl, and heterocycloalkyl, any of which may themselves be optionally substituted.

The term “amino acid,” as used herein, alone or in combination, means a substituent of the form —NRCH(R′)C(O)OH, wherein R is typically hydrogen, but may be cyclized with N (for example, as in the case of the amino acid proline), and R′ is selected from the group consisting of hydrogen, alkyl, heteroalkyl, cycloalkyl, heterocycloalkyl, aryl, heteroaryl, amino, amido, cycloalkylalkyl, heterocycloalkylalkyl, arylalkyl, heteroarylalkyl, aminoalkyl, amidoalkyl, hydroxyalkyl, thiol, thioalkyl, alkylthioalkyl, and alkylthio, any of which may be optionally substituted. The term “amino acid” includes all naturally occurring amino acids as well as synthetic analogues.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄— derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid (oxygen) end, and which may be optionally substituted as defined herein.

The term “O-carbamyl” as used herein, alone or in combination, refers to a —OC(O)NRR′, group, with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(═O)H] and in combination is a —C(═O)— group.

The term “carboxyl” or “carboxyl,” as used herein, refers to —C(═O)OH, O-carboxy, C-carboxy, or the corresponding “carboxylate” anion, such as is in a carboxylic acid salt. An “O-carboxy” group refers to a RC(═O)O— group, where R is as defined herein. A “C-carboxy” group refers to a —C(═O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl radical wherein each cyclic moiety contains from 3 to 12 carbon atom ring members and which may optionally be a benzo fused ring system which is optionally substituted as defined herein. In some embodiments, a cycloalkyl may comprise from from 3 to 7 carbon atoms, or from 5 to 7 carbon atoms. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1.1.1]pentane, camphor, adamantane, and bicyclo[3.2.1]octane.

The term “electrophilic moiety,” as used herein, is used in accordance with its plain ordinary chemical meaning and refers to a chemical group that is electrophilic.

Exemplary electrophilic moieties include, without limitation, unsaturated carbonyl containing compounds such as acrylamides, acrylates, unsaturated (i.e., vinyl) sulfones or phosphates, epoxides, and vinyl epoxides.

The term “ester,” as used herein, alone or in combination, refers to a carboxyl group bridging two moieties linked at carbon atoms (—CRR′C(═O)OCRR′—), where each R and R′ are independent and defined herein.

The term “ether,” as used herein, alone or in combination, typically refers to an oxy group bridging two moieties linked at carbon atoms. “Ether” may also include polyethers, such as, for example, —RO(CH₂)₂O(CH₂)₂O(CH₂)₂OR′, —RO(CH₂)₂O(CH₂)₂OR′, —RO(CH₂)₂OR′, and —RO(CH₂)₂OH.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl, trihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals.

Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized (i.e. bond to 4 groups). The heteroatom(s) O, N and S may be placed at any interior position of the heteroalkyl group. Up to two heteroatoms may be consecutive, such as, for example, —CH₂NHOCH₃. The term heteroalkyl may include ethers.

The term “heteroaryl,” or “heteroaromatic,” as used herein, alone or in combination, refers to 3 to 7 membered unsaturated heteromonocyclic rings, or fused polycyclic rings, each of which is 3 to 7 membered, in which at least one of the fused rings is unsaturated, wherein at least one atom is selected from the group consisting of O, S, and N. In some embodiments, a heteroaryl may comprise from 5 to 7 carbon atoms. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Non-limiting examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

Heteroaryl or heteroaromatic groups can include any number of ring atoms, such as, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl or heteroaromatic groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6- pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl or heteroaromatic groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

The terms “heterocycloalkyl” and, interchangeably, “heterocycle,” or “heterocyclyl” as used herein, alone or in combination, each refer to a saturated, partially unsaturated, or fully unsaturated monocyclic, bicyclic, or tricyclic heterocyclic radical containing at least one heteroatom as ring members, wherein each heteroatom may be independently selected from the group consisting of nitrogen, oxygen, and sulfur. In certain embodiments, a heterocycloalkyl may comprise from 1 to 4 heteroatoms as ring members. In further embodiments, a heterocycloalkyl may comprise from 1 to 2 heteroatoms ring members. In some embodiments, a heterocycloalkyl may comprise from 3 to 8 ring members in each ring. In further embodiments, a heterocycloalkyl may comprise from 3 to 7 ring members in each ring. In yet further embodiments, a heterocycloalkyl may comprise from 5 to 6 ring members in each ring. “Heterocycloalkyl” and “heterocycle” are intended to include sugars, sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycloalkyl groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, epoxy, isoindolinyl, morpholinyl, piperazinyl, pyrrolidinyl, tetrahydropyridinyl, piperidinyl, thiomorpholinyl, and the like. The heterocycloalkyl groups may be optionally substituted unless specifically prohibited.

“Heterocycloalkyl” may refer to a saturated ring system having from 3 to 12 ring members and from 1 to 5 heteroatoms of N, O and S. The heteroatoms can also be oxidized, such as, but not limited to, S(O) and S(O)₂. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, 4, or 5, or 1 to 2, 1 to 3, 1 to 4, 1 to 5, 2 to 3, 2 to 4, 2 to 5, 3 to 4 or 3 to 5. The heterocycloalkyl group can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. The heterocycloalkyl group can include groups such as aziridine, azetidine, pyrrolidine, piperidine, azepane, diazepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline, diazabicycloheptane, diazabicyclooctane, diazaspirooctane or diazaspirononane. Heterocycloalkyl groups can be unsubstituted or substituted. For example, heterocycloalkyl groups can be substituted with C1 6 alkyl or oxo (═O), among many others. Heterocycloalkyl groups can also include a double bond or a triple bond, such as, but not limited to dihydropyridine or 1,2,3,6-tetrahydropyridine.

The heterocycloalkyl groups can be linked via any position on the ring. For example, aziridine can be 1- or 2-aziridine, azetidine can be 1- or 2- azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5- isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

The term “hydrazinyl” as used herein, alone or in combination, refers to two amino groups joined by a single bond, i.e., —N—N—. In general, the hydrazinyl group has optional substitution on at least one NH hydrogen to confer stability.

The term “hydroxamic acid” or its ester as used herein, refers to —C(O)ON(R)O(R′), wherein R and R′ are as defined herein, or the corresponding “hydroxamate” anion, including any corresponding hydroxamic acid salt.

The term “hydroxy,” as used herein, alone or in combination, refers to OH.

The term “hydroxyalkyl,” as used herein, alone or in combination, refers to a hydroxy group attached to the parent molecular moiety through an alkyl group. “Hydroxyalkyl” or “alkylhydroxy” refers to an alkyl group, as defined above, where at least one of the hydrogen atoms is replaced with a hydroxy group. As for the alkyl group, hydroxyalkyl or alkylhydroxy groups can have any suitable number of carbon atoms, such as C₁₋₆. Exemplary C₁₋₄ hydroxyalkyl groups include, but are not limited to, hydroxymethyl, hydroxyethyl (where the hydroxy is in the 1- or 2-position), hydroxypropyl (where the hydroxy is in the 1-, 2- or 3-position), hydroxybutyl (where the hydroxy is in the 1-, 2-, 3- or 4-position), 1,2-dihydroxyethyl, and the like.

The term “imino,” as used herein, alone or in combination, refers to C=NR.

The term “iminohydroxy,” as used herein, alone or in combination, refers to C═N(OH) and it O-ether C═N—OR.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “linking group,” as used herein refers to any nitrogen containing organic fragment that serves to connect the pyrimidine or pyridone core of the compounds disclosed herein to the electrophilic moiety E, as defined herein. Exemplary linking groups include piperazines, aminoalkyls, alkyl- or aryl-based diamines, aminocycloalkyls, amine-containing spirocyclics, any of which may be optionally substituted as defined herein. In some embodiments, linking groups may comprise the substructure L-Q-L′-E wherein Q is a monocyclic 4 to 7 membered ring or a bicyclic, bridged, or fused, or spiro 6-11 membered ring, any of which optionally include one or more nitrogen atoms, E is the electrophilic group, L is bond, C₁₋₆ alkylene, —O—C₀₋₅ alkylene, —S—C₀₋₅ alkylene, or —NH—C₀₋₅ alkylene, and for C₂₋₆ alkylene, —O—C₂₋₅ alkylene, —S—C₂₋₅ alkylene, and NH—C₂_₅ alkylene, one carbon atom of any of the alkylene groups can optionally be replaced with O, S, or NH; and L′ is bond when Q comprises a nitrogen to link to E, otherwise L′ is NR, where R is hydrogen or alkyl.

The term “lower,” as used herein, alone or in combination, means containing from 1 to and including 6 carbon atoms, or from 1 to 4 carbon atoms.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “phosphoamide” as used herein, alone or in combination, refers to a phosphate group [(OH)₂P(═O)O—] in which one or more of the hydroxyl groups has been replaced by nitrogen, amino, or amido.

The term “phosphonate” as used herein, alone or in combination, refers to a group of the form ROP(OR′)(OR)O— wherein R and R′ are selected from the group consisting of hydrogen, alkyl, acyl, heteroalkyl, aryl, cycloalkyl, heteroaryl, and heterocycloalkyl, any of which may themselves be optionally substituted. “Phosphonate” includes “phosphate [(OH)₂P(O)O—] and related phosphoric acid anions which may form salts.

The terms “sulfonate,” “sulfonic acid,” and “sulfonic,” as used herein, alone or in combination, refers to the —SO₃H group and its anion as the sulfonic acid is used in salt formation or sulfonate ester where OH is replaced by OR, where R is not hydrogen, but otherwise is as defined herein, and typically being alkyl or aryl.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(═S)H and in combination is a —C(═S)— group.

The term “N-thiocarbamyl” refers to an ROC(═S)NR′— group, with R and R′ as defined herein.

The term “O-thiocarbamyl” refers to a —OC(═S)NRR′, group with R and R′ as defined herein.

The term “thiocyanato” refers to a —CNS group.

The term “trihalomethanesulfonamido” refers to a X₃CS(═O)₂NR— group with X is a halogen and R as defined herein.

The term “trihalomethanesulfonyl” refers to a X₃CS(═O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

In embodiments of the disclosure, any one of the positions that is understood to have a hydrogen may also exist or understood to be isotopically enriched. In the compounds of this disclosure any atom not specifically designated as a particular isotope is meant to represent any stable isotope of that atom. Obtaining 100% deuteration at any relevant site of a compound in an amount of milligram or greater can be difficult.

Therefore, it is understood that some percentage of hydrogen may still be present, even though a deuterium atom is specifically shown in a chemical structure. Thus, when a chemical structure contains a “D,” the compound represented by the structure is deuterium-enriched at the site represented by “D.” Unless otherwise stated, when a position is designated specifically as “H” or “hydrogen,” the position is understood to have hydrogen at its natural abundance isotopic composition. Also unless otherwise stated, when a position is designated specifically as “D” or “deuterium,” the position is understood to have deuterium at an abundance that is at least 3340 times greater than the natural abundance of deuterium, which is 0.015% (i.e., the term “D” or “deuterium” indicates at least 50.10% incorporation of deuterium). In embodiments, a benzene ring may be optionally exist as —C₆D₅, —C₆DH₄, —C₆D₂H₃, —C₆D₃H₂, and —C₆D₄H. In embodiments, a cyclohexyl group may optionally exist as —C₆D₁₁.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent. A “null” group occurring between two other group may also be understood to be a collapsing of flanking groups. For example, if in —(CH₂)ₓG¹G²G³, the element G² were null, said group would become —(CH₂)ₓG¹G³.

The term “optionally substituted” means the anteceding group or groups may be substituted or unsubstituted. Groups constituting optional substitution may themselves be optionally substituted. For example, where an alkyl group is embraced by an optional substitution, that alkyl group itself may also be optionally substituted. When substituted, the substituents of an “optionally substituted” group may include, without limitation, one or more substituents independently selected from the following groups or a particular designated set of groups, alone or in combination: alkyl, alkenyl, alkynyl, alkanoyl, heteroalkyl, heterocycloalkyl, haloalkyl, haloalkenyl, haloalkynyl, lower perhaloalkyl, perhaloalkoxy, cycloalkyl, phenyl, aryl, aryloxy, alkoxy, haloalkoxy, oxo, acyloxy, carbonyl, carboxyl, alkylcarbonyl, carboxyester, carboxamido, cyano, hydrogen, halogen, hydroxy, amino, alkylamino, arylamino, amido, nitro, thiol, alkylthio, haloalkylthio, perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, carbamate, and urea. Particular subsets of optional substitution include, without limitation: (1) alkyl, halo, and alkoxy; (2) alkyl and halo; (3) alkyl and alkoxy; (4) alkyl, aryl, and heteroaryl; (5) halo and alkoxy; and (6) hydroxyl, alkyl, halo, alkoxy, and cyano. Where an optional substitution comprises a heteroatom-hydrogen bond (—NH-, SH, OH), further optional substitution of the heteroatom hydrogen is contemplated and includes, without limitation optional substitution with alkyl, acyl, alkoxymethyl, alkoxyethyl, arylsulfonyl, alkyl sulfonyl, any of which are further optionally substituted. These subsets of optional substitutions are intended to be merely exemplary and any combination of 2 to 5, or 2 to 10, or 2 to 20 of the groups recited above up to all the group recited above and any subrange in between are contemplated. “Optionally substituted” may include any of the chemical functional groups defined hereinabove and throughout this disclosure. Two optional substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃).

The various optional substitutions need not be the same and any combination of optional substituent groups may be combined. For example, a carbon chain may be substituted with an alkyl group, a halo group, and an alkoxy group. Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, hydroxyl, halogen, alkyl, cycloalkyl, heteroalkyl, aryl, heteroaryl and heterocycloalkyl, any of which may be optionally substituted. Each such R and R′ groups should be understood to be optionally substituted as defined herein. Each incidence of R and R′ should be understood to be independent. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers, axial asymmetry (non-interchanging rotamers), or the like may exist in the compounds of the various embodiments disclosed herein. Such chirality may be designated by the symbols “R” or “S,” depending on the configuration of substituents around the chiral carbon atom or the relevant axis. It should be understood that embodiments encompasses all stereochemical isomeric forms, including diastereomeric, enantiomeric, and epimeric forms, d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the various embodiments disclosed herein may exist as geometric isomers. The various embodiments disclosed herein includes all cis, trans, syn, anti, entgegen (E), and zusammen (Z) isomers as well as the appropriate mixtures thereof. Additionally, compounds may exist as tautomers, including keto-enol tautomers; all tautomeric isomers are embraced by the embodiments disclosed herein.

Additionally, the compounds of the various embodiments disclosed herein can exist in unsolvated as well as solvated forms with pharmaceutically acceptable solvents such as water, ethanol, and the like. In general, the solvated forms are considered equivalent to the unsolvated forms for the purposes of the various embodiments disclosed herein.

The term “bond” refers to a covalent linkage between two atoms, or two moieties when the atoms joined by the bond are considered part of larger substructure. A bond may be single, double, or triple unless otherwise specified. A dashed line between two atoms in a drawing of a molecule indicates that an additional bond may be present or absent at that position.

### 1. Salts of Compounds

The compounds disclosed herein can exist as pharmaceutically acceptable salts, including acid addition salts. Suitable salts include those formed with both organic and inorganic acids. Such acid addition salts will normally be pharmaceutically acceptable.

However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002). It is understood that each of the compounds disclosed herein, and each embodiment of the compounds set forth herein, include pharmaceutically acceptable salts of such compounds.

The term “pharmaceutically acceptable salt,” as used herein, represents salts or zwitterionic forms of the compounds disclosed herein which are water or oil-soluble or dispersible and pharmaceutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the various embodiments disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the various embodiments disclosed herein contemplates sodium, potassium, magnesium, and calcium salts of the compounds disclosed herein, and the like.

Basic addition salts can be prepared during the final isolation and purification of the compounds by reacting a carboxyl group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

### C. Treatment-Related Definitions

The term “disease” as used herein is intended to be generally synonymous, and is used interchangeably with, the terms “disorder” and “condition” (as in medical condition), in that all reflect an abnormal condition of the body or of one of its parts that impairs normal functioning and is typically manifested by distinguishing signs and symptoms.

As used herein, the term “cancer” refers to all types of cancer, neoplasm or malignant tumors found in mammals (e.g., but not limited to, humans), including leukemia, lymphomas, carcinomas and sarcomas. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi′ssarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

“Ras associated cancer” (also referred to herein as “Ras related cancer”) refers to a cancer caused by aberrant Ras activity or signaling. A “cancer associated with aberrant K-Ras activity” (also referred to herein as “K-Ras related cancer”) is a cancer caused by aberrant K-Ras activity or signaling (e.g. a mutant K-Ras). K-Ras related cancers may include lung cancer, non-small cell lung cancer, breast cancer, leukemia, pancreatic cancer, colon cancer, colorectal cancer. Other cancers that are associated with aberrant activity of one or more of Ras, K-Ras, H-Ras, N-Ras, mutant K-Ras (including K-Ras G12C, K-Ras G12V, K-Ras G13C, K-Ras G12D, K-Ras G13D mutants), mutant N-Ras, and mutant H-Ras are well known in the art, including G12C in both N-Ras and H-Ras, and determining such cancers are within the skill of a person of skill in the art.

The term term “administer (or administering) a Ras inhibitor” means administering a compound that inhibits the activity or level (e.g. amount) or level of a signaling pathway of one or more Ras proteins (e.g. a Ras inhibitor, K-Ras inhibitor, N-Ras inhibitor, H-Ras inhibitor, mutant K-Ras inhibitor, K-Ras G12C inhibitor, K-Ras G12V inhibitor, K-Ras G13C inhibitor, K-Ras G12D inhibitor, K-Ras G13D inhibitor) to a subject. Administration may include, without being limited by mechanism, allowing sufficient time for the Ras inhibitor to reduce the activity of one or more Ras proteins or for the Ras inhibitor to reduce one or more symptoms of a disease (e.g. cancer, wherein the Ras inhibitor may arrest the cell cycle, slow the cell cycle, reduce DNA replication, reduce cell replication, reduce cell growth, reduce metastasis, or cause cell death). The term “administer (or administering) a K-Ras inhibitor” means administering a compound that inhibits the activity or level (e.g. amount) or level of a signaling pathway of one or more K-Ras proteins (K-Ras, mutant K-Ras, K-Ras G12C, K-Ras G12V, K-Ras G12D, K-Ras G13C, K-Ras G13D). In embodiments, the administering does not include administration of any active agent other than the recited active agent.

The term “associated” or “associated with” in the context of a substance or substance activity or function associated with a disease (e.g. Ras (e.g., human K-Ras or human H-Ras) activity, a protein associated disease, a cancer associated with aberrant Ras activity, K-Ras associated cancer, mutant K-Ras associated cancer, activated K-Ras associated cancer, K-RasG12C associated cancer, K-Ras G12V associated cancer, K-Ras G13C associated cancer, K-Ras G12D associated cancer, K-Ras G13D associated cancer) means that the disease (e.g. cancer) is caused by (in whole or in part), or a symptom of the disease is caused by (in whole or inpart) the substance or substance activity or function. For example, a cancer associated with aberrant Ras activity or function may be a cancer that results (entirely or partially) from aberrant Ras activity or function (e.g. enzyme activity, protein-protein binding, signaling pathway) or a cancer wherein a particular symptom of the disease is caused (entirely or partially) by aberrant Ras activity or function. As used herein, what is described as being associated with a disease, if a causative agent, could be a target for treatment of the disease. For example, a cancer associated with aberrant Ras activity or function or a Ras associated cancer, may be treated with a Ras modulator or Ras inhibitor, in the instance where increased Ras activity or function (e.g., signaling pathway activity) causes the cancer. For example, a cancer associated with K-Ras G12C may be a cancer that a subject with K-Ras G12C is at higher risk of developing as compared to a subject without K-Ras G12C. For example, a cancer associated with K-Ras G12V may be a cancer that a subject with K-Ras G12V is at higher risk of developing as compared to a subject without K-Ras G12V.

The term “Ras” refers to one or more of the family of human Ras GTPase proteins (e.g. K-Ras, H-Ras, N-Ras). The term “K-Ras” refers to the nucleotide sequences or proteins of human K-Ras (e.g. human K-Ras4A (NP_203524.1), human K-Ras4B (NP_004976.2), or both K-Ras4A and K-Ras4B). The term “K-Ras” includes both the wild-type form of the nucleotide sequences or proteins as well as any mutants thereof. In some embodiments, “K-Ras” is wild-type K-Ras. In some embodiments, “K-Ras” is one or more mutant forms. The term “K-Ras” XYZ refers to a nucleotide sequence or protein of a mutant K-Ras wherein the Y numbered amino acid of K-Ras that has an X amino acid in the wildtype instead has a Z amino acid in the mutant (e.g. K-Ras G12C has a G in wildtype protein but a C in the K-Ras G12C mutantprotein). In some embodiments K-Ras refers to K-Ras4A and K-Ras4B. In some embodiments, K-Ras refers to K-Ras4A. In some embodiments, K-Ras refers to K-Ras4B (e.g., NM_004985.4 or NP_004976.2). In some embodiments, K-Ras refers to the protein including (e.g., consisting of) the amino acid sequence below or including the sequence below with one or more mutations (e.g., G12C, G12V, or G13C):

(SEQ ID NO: 1)
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHHYRE
QIKRVKDSEDVPMVLVGNKCDLPSRTVDTKQAQDLARSYGIP
FIETSAKTRQGVDDAFYTLVREIRKHKEK

In some embodiments, K-Ras refers to the protein including (e.g., consisting of) the amino acid sequence below or including (e.g., consisting of) the sequence below with one or more mutations (e.g., G12C, G12V, or G13C):

(SEQ ID NO: 2)
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHHYRE
QIKRVKDSEDVPMVLVGNKCDLPSRTVDTKQAQDLARSYGIPFIETSAK
TRQGVDDAFYTLVREIRKHKEKMSKDGKKKKKKSKTKCVIM

1 mteyklvvvg aggvgksalt iqlignhfvd eydptiedsy rkqvvidget clldildtag 61 qeeysamrdq ymrtgegflc vfainntksf edihhyreqi krvkdsedvp mvlvgnkcdl 121 psrtvdtkqa qdlarsygip fietsaktrq gvddafytly reirkhkekm skdgkkkkkk 181 sktkcvim (SEQ ID NO:3)

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“K-RAS inhibitor” is used herein to refer to a compound that exhibits an IC₅₀ with respect to K-RAS activity of no more than about 100 mM and more typically not more than about 50 mM, as measured in the K-RAS assay described generally hereinbelow. “IC₅₀” is that concentration of inhibitor that reduces the activity of an enzyme (e.g., K-RAS) to half-maximal level. Compounds of the various embodiments disclosed herein have been discovered to exhibit inhibition against oncogenic mutant K-RAS isoforms. In some embodiments, compounds will exhibit an IC₅₀ with respect to oncogenic mutant K-RAS of no more than about 10 mM; in further embodiments, compounds will exhibit an IC₅₀ with respect to K-RAS of no more than about 5 mM; in yet further embodiments, compounds will exhibit an IC₅₀ with respect to K-RAS of not more than about 1 mM, as measured in the K-RAS assay described herein. In yet further embodiments, compounds will exhibit an IC₅₀ with respect to K-RAS of not more than about 200 nM. Without being bound by theory, in some embodiments, the K-RAS inhibitor is an irreversible inhibitor by way of covalent bond formation to the cysteine at the G12C mutation site.

The phrase “therapeutically effective” is intended to qualify the amount of active ingredients used in the treatment of a disease or disorder. This amount will achieve the goal of reducing or eliminating the the disease or disorder.

As used herein, reference to “treatment” of a subject is intended to include prophylaxis. The term “subject” means all mammals, including humans. Examples of subjects include humans, cows, dogs, cats, goats, sheep, pigs, and rabbits. In some embodiments, the subject is a human.

The term “prodrug” refers to a compound that is made active in vivo through chemical reaction in vivo thereby releasing an active compound. Compounds disclosed herein can be modified to exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the active compound. Additionally, prodrugs can be converted to the active compounds by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the active compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug is a compound which is administered as an ester (the “prodrug”), which is then metabolically hydrolyzed to the carboxylic acid, as the active entity. Additional examples include peptidyl derivatives of a compound. The term “therapeutically acceptable prodrug,” refers to those prodrugs or zwitterions which are suitable for use in contact with the tissues of subjects without undue toxicity, irritation, and allergic response, are commensurate with a reasonable benefit/risk ratio, and are effective for their intended use.

### III. Compound Embodiments

### A. Genus I—General

In some embodiments, there are provided compounds of Formula (I):

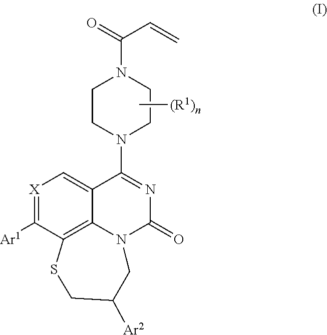

wherein:
each R¹ is independently methyl or cyanomethyl;
n is an integer from 0 to 2;
X is —CCF₃ or —C-halogen;
Ar¹ is aryl or heteroaryl, optionally substituted with one or more alkyl, halogen, hydroxy or amino; and
   Ar² is a C-linked aryl or heteroaryl, optionally substituted with alkyl or halogen.

In some embodiments, Ar¹ is phenyl optionally substituted with one or more halogens.

In one or more of the preceding embodiments, X is —CCl or —CCF₃.

In one or more of the preceding embodiments, Ar¹ is a phenyl substituted with one or more halogens.

In one or more of the preceding embodiments, n is 2 and each R¹ is methyl.

In one or more of the preceding embodiments, Ar² is selected from:

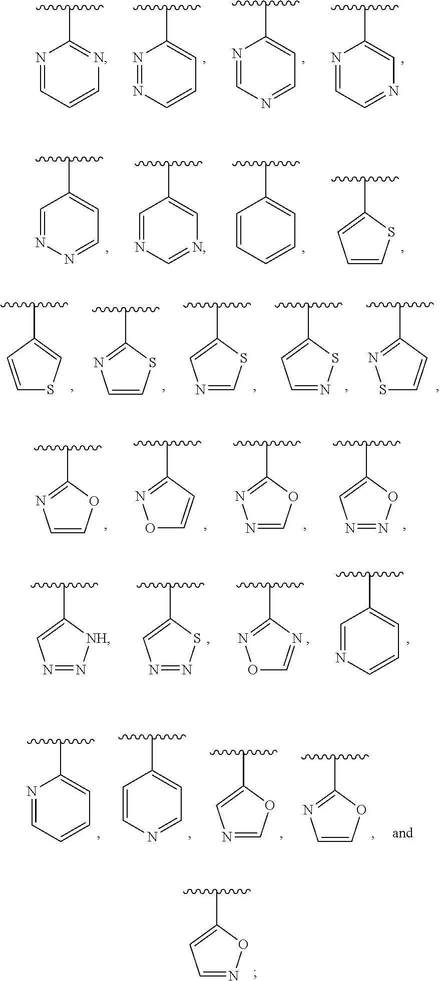

any of which are optionally substituted with halogen.

In embodiments, there are provided further compounds:

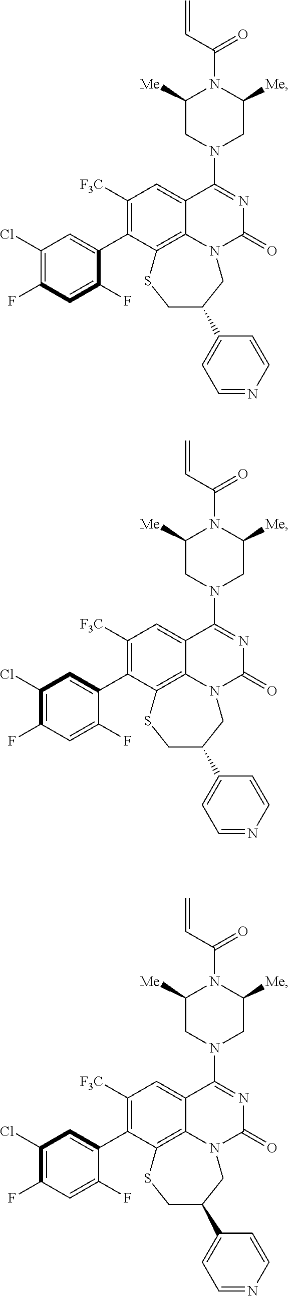

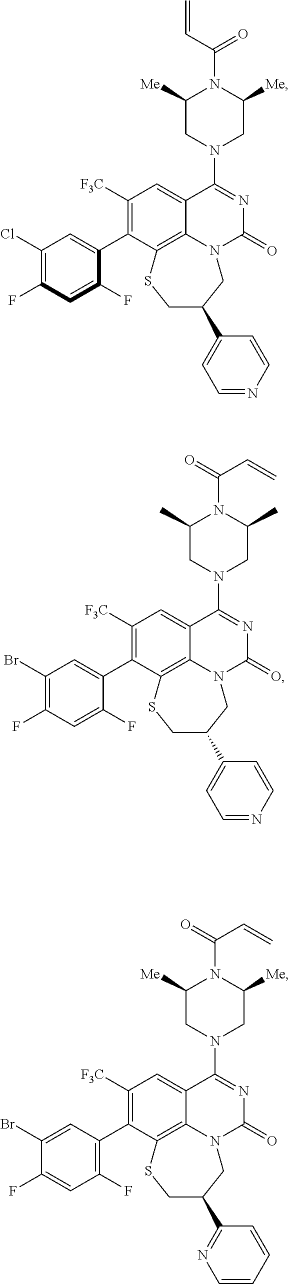

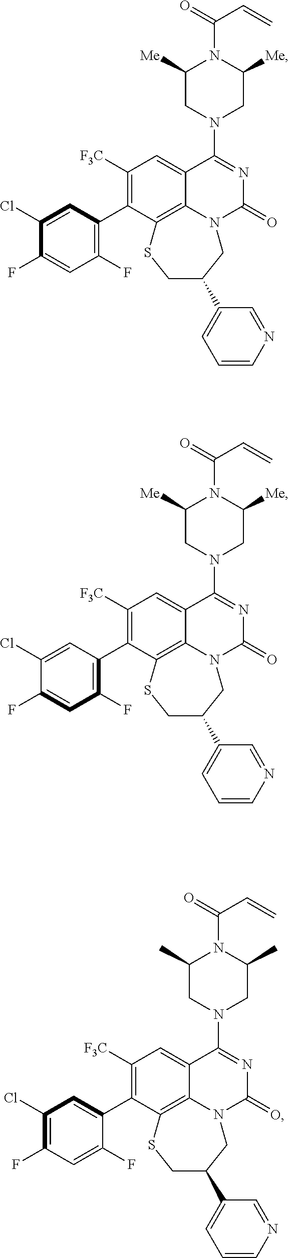

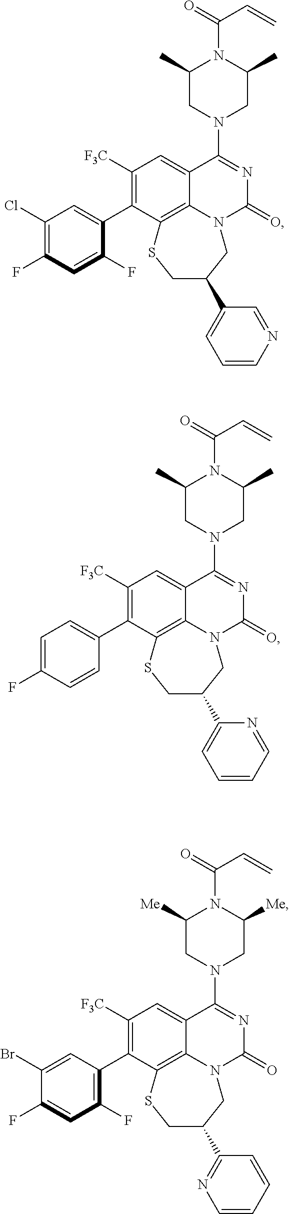

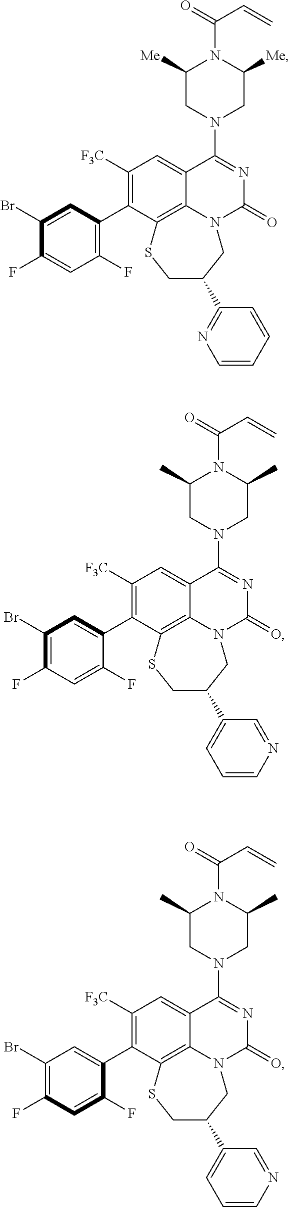

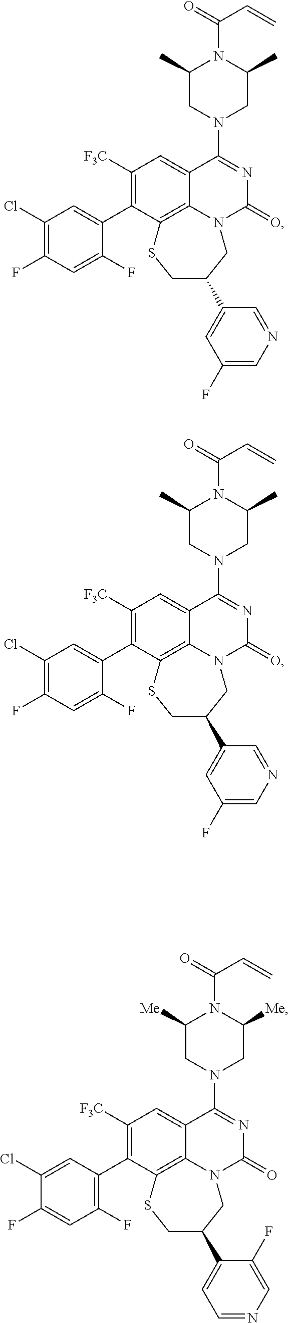 [SMILES: C=CC(=O)N1[C@H](C)CN(c2nc(=O)n3c4c(c(-c5cc(Cl)c(F)cc5F)c(C(F)(F)F)cc24)SC[C@@H](c2cncc(F)c2)C3)C[C@@H]1C ; C=CC(=O)N1[C@H](C)CN(c2nc(=O)n3c4c(c(-c5cc(Cl)c(F)cc5F)c(C(F)(F)F)cc24)SC[C@H](c2cncc(F)c2)C3)C[C@@H]1C ; C=CC(=O)N1[C@H](C)CN(c2nc(=O)n3c4c(c(-c5cc(Cl)c(F)cc5F)c(C(F)(F)F)cc24)SC[C@H](c2ccncc2F)C3)C[C@@H]1C]

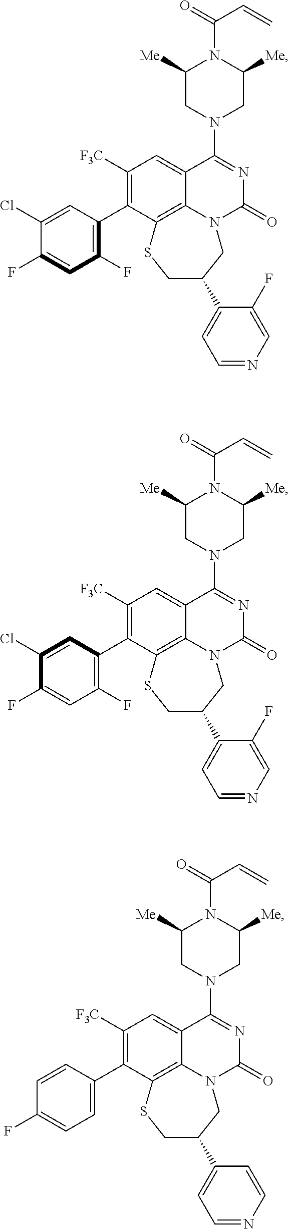

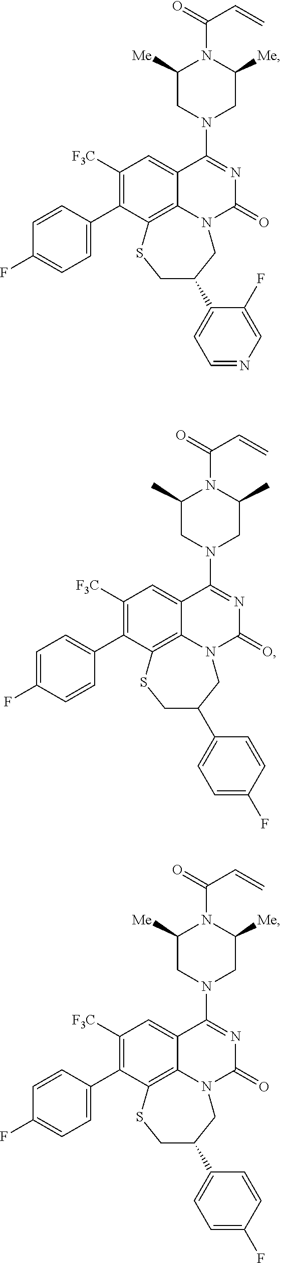 [SMILES: C=CC(=O)N1[C@H](C)CN(c2nc(=O)n3c4c(c(-c5ccc(F)cc5)c(C(F)(F)F)cc24)SC[C@@H](c2ccncc2F)C3)C[C@@H]1C ; C=CC(=O)N1[C@H](C)CN(c2nc(=O)n3c4c(c(-c5ccc(F)cc5)c(C(F)(F)F)cc24)SCC(c2ccc(F)cc2)C3)C[C@@H]1C ; C=CC(=O)N1[C@H](C)CN(c2nc(=O)n3c4c(c(-c5ccc(F)cc5)c(C(F)(F)F)cc24)SC[C@@H](c2ccc(F)cc2)C3)C[C@@H]1C]

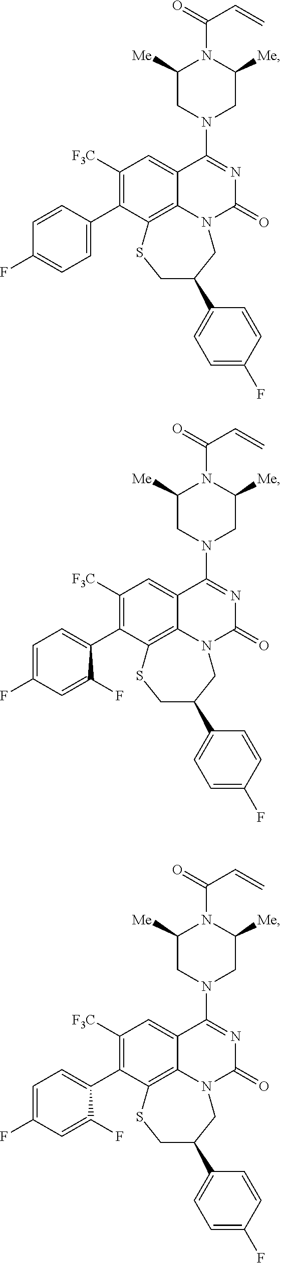

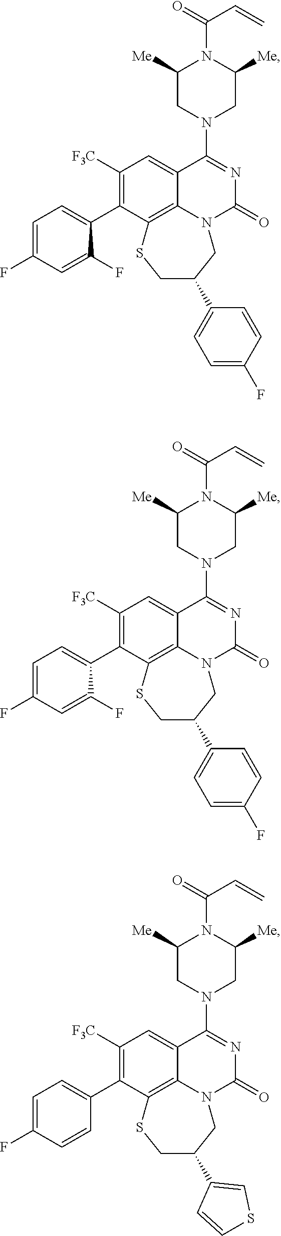

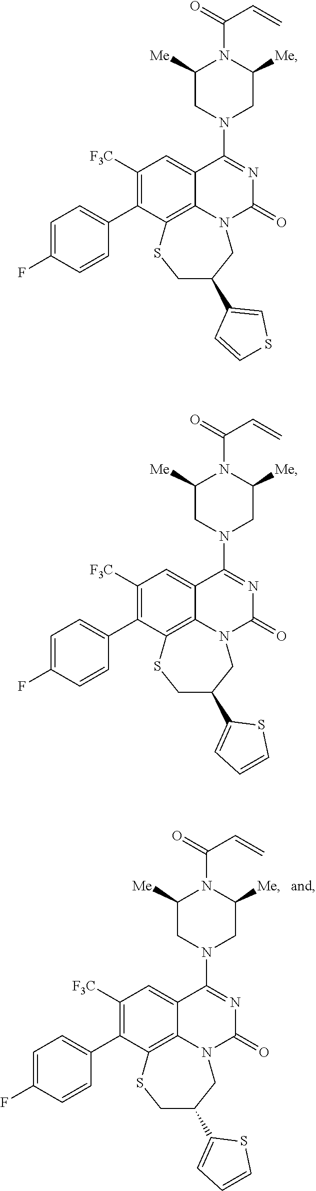

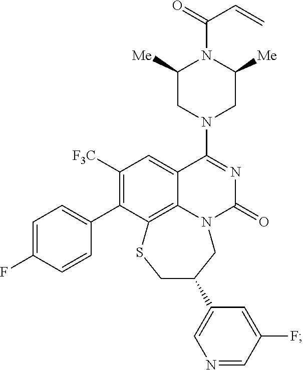 [SMILES: C=CC(=O)N1[C@H](C)CN(c2nc(=O)n3c4c(c(-c5ccc(F)cc5)c(C(F)(F)F)cc24)SC[C@@H](c2cncc(F)c2)C3)C[C@@H]1C]

or a pharmaceutically acceptable salt thereof.

### IV. General Synthetic Methods for Preparing Compounds

The following schemes can be used to practice the various embodiments disclosed herein. It will be understood that these schemes are merely exemplary and that they provide ready access to core structures with variable functionality.

### V. Modes of Administration

While it may be possible for the compounds disclosed herein to be administered as the raw chemical, it is also possible to present them as a pharmaceutical composition (i.e., as a formulation). Accordingly, provided herein are pharmaceutical compositions which comprise one or more of the compounds disclosed herein, or one or more pharmaceutically acceptable salts, esters, prodrugs, amides, or solvates thereof, together with one or more pharmaceutically acceptable carriers and optionally one or more other therapeutic ingredients. The carrier(s) should be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The pharmaceutical compositions may include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The pharmaceutical composition may conveniently be presented in unit dosage form and may be prepared by any of the methods well known in the art of pharmacy. Typically, these methods include the step of bringing into association a compound disclosed herein or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Pharmaceutical compositions of the various embodiments disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical compositions that can be used orally include tablets. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration.

The compounds disclosed herein may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

### Dosage

The compounds disclosed herein may be administered orally or via injection at a dose of from 0.1 to 500 mg/kg per day. A common dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds disclosed herein can be administered in various modes, e.g. orally, topically, or by injection. The precise amount of compound administered to a subject will be the responsibility of the attendant physician. The specific dose level for any particular subject will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer a In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound of the various embodiments disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

### VI. Methods of Treatment

In an aspect, embodiments herein provide a method of treating a subject with cancer comprising administering to the subject an amount of a compound of the various embodiments disclosed herein or pharmaceutically acceptable salt thereof. In embodiments, the cancer comprises a K-Ras G12 mutation. In embodiments, the G12 mutatoin is G12C. In embodiments, the cancer is one or more of pancreatic, lung, and colorectal cancer. In embodiments, the pancreatic cancer is pancreatic ductal adenocarcinoma (PDAC). In further embodiments, the cancer is a CNS cancer. In embodiments, the CNS cancer is a primary cancer. In embodiments, the primary cancer comprises one or more of a glioma, meningioma, medulloblastoma, ganblioglioma, schwannoma, and craniopharyngioma. In embodiments, the glioma comprises one or more of an astrocytoma, a glioblastoma, an oligodendroglioma, and a ependymoma. In embodiments, the CNS cancer comprises a metastatic or secondary cancer. In further embodiments, the CNS cancer comprises a cancer metastasized from one or more of melanoma, breast cancer, colon cancer, kidney cancer, nasopharyngeal cancer, leukemia, lymphoma, myeloma, and other of unknown primary site.

In another aspect, embodiments herein provide methods for treating K-RAS-mediated disorders in a human or animal subject in need of such treatment comprising administering to said subject an amount of a compound of the various embodiments disclosed herein effective to reduce or prevent said disorder in the subject optionally in combination with at least one additional agent for the treatment of said disorder that is known in the art. In a related aspect, the various embodiments disclosed herein provides therapeutic compositions comprising at least one compound of the various embodiments disclosed herein in combination with one or more additional agents for the treatment of K-RAS-mediated disorders. In some such embodiments, the K-RAS-mediated disease is cancer and the K-RAS presents in an oncogenic mutated form.

Compounds disclosed herein may be useful in treating K-RAS-mediated disease, disorders and conditions. In some embodiments, the compounds disclosed herein may be used in treating cancer, as disclosed hereinabove. In some such embodiments, the type of cancer may depend on presentation of a particular type of oncogenic mutation of K-RAS. For example, in some embodiments oncogenic K-RAS mutations may be tied to human cancer of the pancreas, lung, and/or colon.

### 1. Combination Therapies

Compounds disclosed herein may be used in combination therapies. For example, the compounds disclosed herein may be used in combination with inhibitors of mammalian target of rapamycin (mTOR), insulin growth factor 1 receptor (IGF1R), and combinations thereof. Such combination therapies may be particularly suited to certain cancer types such as lung cancer. See Molinas-Arcas et al. Sci. Trans. Med. 18 Sep. 2019 11:510 eaaw7999 at stm.sciencemag.org/content/11/510/eaaw7999. Compounds disclosed herein may be combined with modulators the ULK family of proteins, which regulate autophagy. Other compounds of interest in combination therapy include inhibitors of SHP2. Other SHP2 inhibitors include those disclosed in WO2016/203404, WO2018/136264, WO2018/057884, WO2019/067843, WO2019/183367, WO2016/203405, WO2019/051084, WO2018/081091, WO2019/165073, WO2017/216706, WO2018/218133, WO2019/183364, WO 2020061103, and WO2020061101. All references and patent applications, including compositions, methods of using, and methods of making compounds disclosed therein are incorporated herein by reference in their entirety.

In embodiments, compounds disclosed herein may be combined with an EGFR inihibitor. In embodiments, the EGFR inhibitor is selective for a mutant EGFR, including, without limitation, C797X, L718Q, G724S, S768I, G719X, L792X, G796X, T263P, A289D/V, G598V, and EGFRvIII high expression. In embodiments, the combination therapy with EGFR agents tracked by mutation and indication are shown in Table CT-1 below.

TABLE 1

| Mutation | Indication | EGFR agent |
|---|---|---|
| mEGFR | NSCLC | osimertinib |
| mEGFR | NSCLC | afatinib |
| mEGFR | NSCLC | erlotinib |
| mEGFR | NSCLC | gefitinib |
| mEGFR | NSCLC | lazertinib |
| mEGFR | NSCLC | nazartinib |
| mEGFR | NSCLC | dacomitinib |
| mEGFR | NSCLC | BLU-945 |
| mEGFR | NSCLC | icotinib |
| wtEGFR | Esophageal/CRC | cetuximab |
| wtEGFR | CRC | paninitumab |
| wtEGFR | NSCLC | amivantamab |
| wtHER2/wtEGFR | Breast cancer | lapatinib |
| wtHER2/wtEGFR | Breast cancer | neratinib |
| wtEGFR | NSCLC | zorifertinib |
| mEGFR | NSCLC | mobicertinib |

EGFR inhibitors include those disclosed in U.S. Pat. Nos. 5,747,498, 8,946,235, and 9,732,058, WO2002030926, US 20040048880, US20050165035, and WO2019067543. All patents and applications, including compositions, methods of using, and methods of making compounds disclosed therein are incorporated herein by reference in their entirety.

Other combination therapies based on target biomarkers are shown below in Table CT-2.

TABLE 2

| | Cancer | | Combination |
|---|---|---|---|
| Biomarker(s) | Type | Target | Agent |
| KRAS G12C | Solid | KRAS G12C | AMG 510 |
| | tumors | | |
| KRAS G12C | Solid | KRAS G12C | MRTX849 |
| | tumors | | |
| KRAS G12C | Solid | KRAS G12C | GDC-6036 |
| | tumors | | |
| BRAF V600E | CRC/ | BRAF V600E | encorafenib |
| | NSCLC | | |
| BRAF V600E | CRC/ | BRAF V600E | dabrafenib |
| | NSCLC | | |
| BRAF V600E | CRC/ | BRAF V600E | encorafenib and |
| | NSCLC | and MEK | binimetinib |
| BRAF V600E | CRC/ | BRAF V600E | dabrafenib and |
| | NSCLC | and MEK | trametinib |
| RB1 functional | Solid | CDK4 and | palbociclib |
| | tumors | CDK6 | |
| RB1 functional | Solid | CDK4 and | abemaciclib |
| | tumors | CDK6 | |
| RB1 functional | Solid | CDK4 and | ribociclib |
| | tumors | CDK6 | |
| RTK and/or RAS Driven | Solid | SHP2 | TNO155 |
| | tumors | | |
| RTK and/or RAS Driven | Solid | SHP2 | RMC-4630 |
| | tumors | | |
| RTK and/or RAS Driven | Solid | SHP2 | JAB-3068 |
| | tumors | | |
| RTK and/or RAS Driven | Solid | SHP2 | JAB-3312 |
| | tumors | | |
| RTK and/or RAS Driven | Solid | SHP2 | RLY-1971 |
| | tumors | | |
| RTK, RAS, BRAF, and/or | Solid | ERK | ulixertinib |
| MEK driven | tumors | | |
| RTK, RAS, BRAF, and/or | Solid | ERK | ASN007 |
| MEK driven | tumors | | |
| RTK, RAS, BRAF, and/or | Solid | ERK | LY3214996 |
| MEK driven | tumors | | |
| RTK, RAS, BRAF, and/or | Solid | ERK | LTT462 |
| MEK driven | tumors | | |
| RTK, RAS, and/or BRAF | Solid | MEK | trametinib |
| | tumors | | |
| RTK, RAS, and/or BRAF | Solid | MEK | binimetinib |
| | tumors | | |
| RTK, RAS, and/or BRAF | Solid | MEK | cobimetinib |
| | tumors | | |
| RTK, RAS, and/or BRAF | Solid | MEK | selumetinib |
| | tumors | | |
| MET-driven | Solid | MET | capmatinib |
| | tumors | | |
| MET-driven | Solid | MET | crizotinib |
| | tumors | | |
| MET-driven | Solid | MET | savolitinib |
| | tumors | | |

The second agent of the pharmaceutical combination formulation or dosing regimen may have complementary activities to the compounds disclosed herein such that they do not adversely affect each other. The compounds may be administered together in a unitary pharmaceutical composition or separately. In one embodiment a compound or a pharmaceutically acceptable salt can be co-administered with a cytotoxic agent to treat proliferative diseases and cancer.

The term “co-administering” refers to either simultaneous administration, or any manner of separate sequential administration, of a compound disclosed herein or a salt thereof, and a further active pharmaceutical ingredient or ingredients, including cytotoxic agents and radiation treatment. If the administration is not simultaneous, the compounds are administered in a close time proximity to each other. Furthermore, it does not matter if the compounds are administered in the same dosage form, e.g. one compound may be administered topically and another compound may be administered orally.

Those additional agents may be administered separately from an inventive compound-containing composition, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this disclosure. For example, a compound disclosed herein may be administered with another therapeutic agent simultaneously or sequentially in separate unit dosage forms or together in a single unit dosage form. Accordingly, the present disclosure provides a single unit dosage form comprising a compound of Formula I, an additional therapeutic agent, and a pharmaceutically acceptable carrier, adjuvant, or vehicle.

The amount of both the compound and additional therapeutic agent (in those compositions which comprise an additional therapeutic agent as described above) that may be combined with the carrier materials to produce a single dosage form will vary depending upon the host treated and the particular mode of administration. In certain embodiments, compositions of this disclosure are formulated such that a dosage of between 0.01-100 mg/kg body weight/day of an inventive can be administered.

Typically, any agent that has activity against a disease or condition being treated may be co-administered. Examples of such agents can be found in Cancer Principles and Practice of Oncology by V. T. Devita and S. Hellman (editors), 6 edition (Feb. 15, 2001), Lippincott Williams & Wilkins Publishers. A person of ordinary skill in the art would be able to discern which combinations of agents would be useful based on the particular characteristics of the drugs and the disease involved.

In one embodiment, the treatment method includes the co-administration of a compound disclosed herein or a pharmaceutically acceptable salt thereof and at least one cytotoxic agent. The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signalling inhibitors; HDAC inhibitors, proteasome inhibitors; and inhibitors of cancer metabolism.

“Chemotherapeutic agent” includes chemical compounds useful in the treatment of cancer. Examples of chemotherapeutic agents include erlotinib (TARCEVA*, Genentech/OSI Pharm.), bortezomib (VELCADE®, Millennium Pharm.), disulfiram, epigallocatechin gallate, salinosporamide A, carfilzomib, 17-AAG(geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®., Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1 and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the disclosure include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG₁ λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-IC11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTal/P2 blockers such as Anti-lymphotoxin alpha (LTa); radioactive isotopes (e.g., At²¹¹, I³¹I 1²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In certain embodiments, chemotherapeutic agents include, but are not limited to, doxorubicin, dexamethasone, vincristine, cyclophosphamide, fluorouracil, topotecan, interferons, platinum derivatives, taxanes (e.g., paclitaxel, docetaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, trimetrexate, metoprine, cyclosporine, daunorubicin, teniposide, amphotericin, alkylating agents (e.g., chlorambucil), 5-fluorouracil, campthothecin, cisplatin, metronidazole, and imatinib mesylate, among others. In other embodiments, a compound disclosed herein is administered in combination with a biologic agent, such as bevacizumab or panitumumab.

In certain embodiments, compounds disclosed herein, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG live, bevacuzimab, fluorouracil, bexarotene, bleomycin, bortezomib, busulfan, calusterone, capecitabine, camptothecin, carboplatin, carmustine, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, elotinib, estramustine, etoposide phosphate, etoposide, exemestane, filgrastim, floxuridine, fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

Chemotherapeutic agents also include treatments for Alzheimer's Disease such as donepezil hydrochloride and rivastigmine; treatments for Parkinson's Disease such as L-DOPA/carbidopa, entacapone, ropinrole, pramipexole, bromocriptine, pergolide, trihexephendyl, and amantadine; agents for treating multiple sclerosis (MS) such as beta interferon (e.g., Avonex® and Rebif®), glatiramer acetate, and mitoxantrone; treatments for asthma such as albuterol and montelukast sodium; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

Additionally, chemotherapeutic agents include pharmaceutically acceptable salts, acids or derivatives of any of chemotherapeutic agents, described herein, as well as combinations of two or more of them.

### VII. Examples

The following Examples are provided to illustrate exemplary embodiments of the compounds disclosed herein and their preparation.

Various starting materials and other reagents were purchased from commercial suppliers, such as Aldrich Chemical Company, and used without further purification, unless indicated otherwise. Compounds are prepared according to the exemplary procedures provided herein and modifications thereof known to those of skill in the art. The following abbreviations are used throughout the Examples: “Ac” means acetyl, “AcO” or “OAc” means acetoxy, “ACN” means acetonitrile, “aq” means aqueous, “atm” means atmosphere(s), “BOC”, “Boc” or “boc” means N-tert-butoxycarbonyl, “Bn” means benzyl, “Bu” means butyl, “nBu” means normal-butyl, “tBu” means tert-butyl, “Cbz” means benzyloxycarbonyl, “DBU” means 1,8-diazabicyclo[5.4.0]undec-7-ene, “DCM” (CH₂Cl₂) means methylene chloride/dichloromethane, “de” means diastereomeric excess, “DEA” means diethylamine, “DIPEA” means diisopropylethyl amine, “DMA” means N,N-dimethylacetamide, “DMAP” means 4-dimethylaminopyridine, “DMF” means N,N-dimethyl formamide, “DMSO” means dimethylsulfoxide, “DPPP” means 1,3-bis(diphenylphosphino)propane, “ee” means enantiomeric excess, “Et” means ethyl, “EtOAc” means ethyl acetate, “EtOH” means ethanol, “HATU” means 1-[bis(dimethylamino)methylene]-1H-1,2,3-triazolo[4,5-b]pyridinium 3-oxid hexafluorophosphate, “HOAc” or “AcOH” means acetic acid, “i-Pr” means isopropyl, “IPA” means isopropyl alcohol, “LDA” means lithium diisopropylamide, “LiHMDS” or “LHMDS” means lithium hexamethyldisilazide, “Me” means methyl, “MeOH” means methanol, “MgSO₄” means magnesium sulphate, “MS” means mass spectrometry, “MTBE” means methyl tert-butyl ether, Na₂SO₄” means sodium sulphate, “NMP” means 1-methyl 2-pyrrolidinone, “Ph” means phenyl, “sat.” means saturated, “SFC” means supercritical fluid chromatography, “TBME” or “MTBE” means tert-butyl methyl ether, “TEA” means triethyl amine, “TFA” means trifluoroacetic acid, “THF” means tetrahydrofuran, “TLC” means thin layer chromatography, “Rf” means retention fraction, “about” means approximately, “rt” means retention time, “RT” means room temperature, “h” means hours, “min” means minutes, “N” means Normal, “M” means molar, “mL” means milliliter, “mmol” means millimoles, “mol” means micromoles, “eq.” means equivalent, “° C.” means degrees Celsius, and “Pa” means pascals. ¹H-NMR spectra are reported in ppm, and were obtained as CDCl₃ solutions (7.25 ppm), DMSO-D₆ solutions (2.50 ppm), or CD₃OD solutions (3.4 ppm and 4.8 ppm), any may have used internal tetramethylsilane (0.00 ppm) as an internal standard when appropriate. Other NMR solvents were used as needed. When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz).

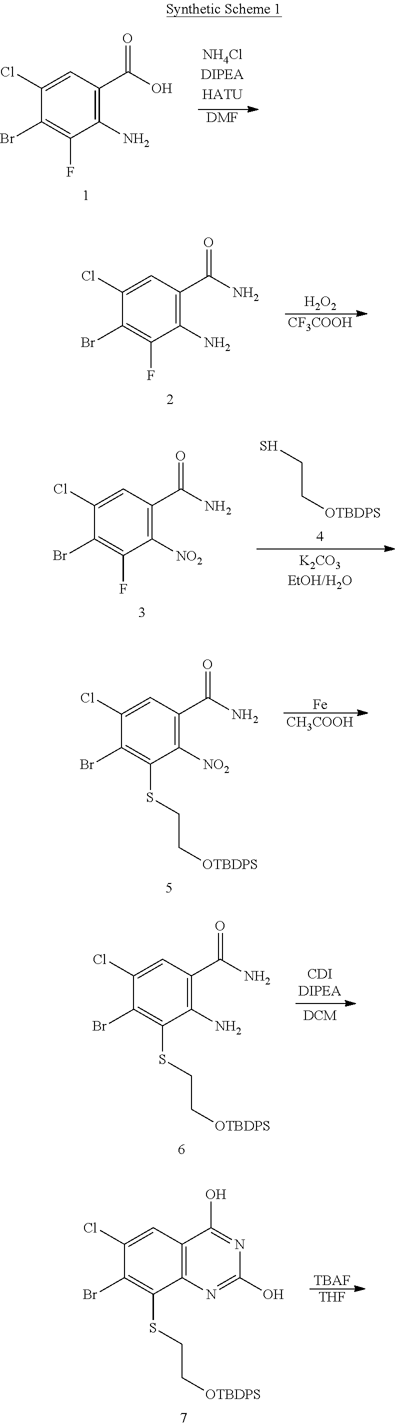

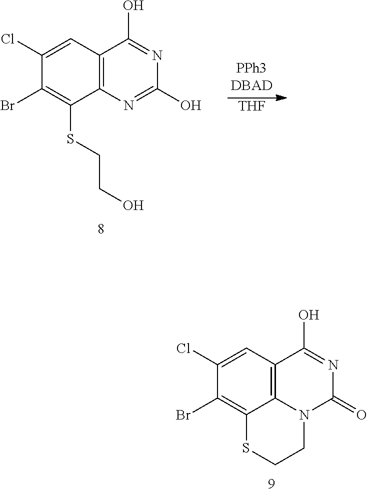 [SMILES: OCCSc1c(Br)c(Cl)cc2c(O)nc(O)nc12 ; O=c1nc(O)c2cc(Cl)c(Br)c3c2n1CCS3 ; "THF"]

### 2. Preparation of Compound 2

To a solution of 2-amino-4-bromo-5-chloro-3-fluorobenzoic acid (2.68 g, 10 mmol, 1.0 eq.) in DMF (27 mL) was added DIPEA (6.45 g, 50 mmol, 10 eq.), NH₄Cl (3.20 g, 60 mmol, 6 eq.) and HATU (7.6 g, 20 mmol, 2 eq.) under N₂ atmosphere at RT. Then the reaction mixture was stirred for 2 hours, diluted with MTBE (100 mL), washed with 0.5N HCl aq. (50 mL), brine (50 mL) and dried over Na₂SO₄. The organic layer was concentrated in vacuo. The residue obtained was purified by a chromatography (0-50% EtOAc/petroleum ether) to provide the product 2 as a yellow solid (2.3 g, yield: 86%). LC-MS: [M+H]⁺=267

### 3. Preparation of Compound 3

To a solution of 2-amino-4-bromo-5-chloro-3-fluorobenzamide (2.67 g, 10 mmol, 1.0 eq.) in CF₃COOH (27 mL) was added hydrogen peroxide (5.7 g, 5 0 mmol, 5 eq.). The reaction was stirred at 50° C. for 0.5 hour. Then diluted with MTBE (150 mL), washed with water (100 mL), brine (100 mL), and then dried over Na₂SO₄. The organic layer was concentrated in vacuo, the residue obtained was purified by a chromatography (0-100% EtOAc/petroleum ether) to provide the product 3 as a yellow solid (1.8 g, yield: 60%). LC-MS: [M+H]⁺=297

### 4. Preparation of Compound 5

To a solution of 4-bromo-5-chloro-3-fluoro-2-nitrobenzamide (3.0 g, 10 mmol, 1.0 eq) in EtOH (30 ml) and water (6 mL) was added 2-((tert-butyldiphenylsilyl)oxy)ethane-1-thiol (3.2 g, 10 mmol, 1.0 eq), potassium carbonate (4.2 g, 30 mmol, 3.0 eq). Then the reaction mixture was stirred at 50° C. for 2 hours. The solvent was removed to afford 6.5 g of the crude product which was used in the subsequent step without further purification. LC-MS: [M+H]⁺=593

### 5. Preparation of Compound 6

To a solution of compound 5 (6.5 g crude, 10 mmol, 1.0 eq.) in CH₃COOH (120 mL) was added Iron powder (2.8 g, 50 mmol, 5 eq.). The reaction mixture was stirred at 50° C. for 2 h. After filtration, the collected solid was washed with EtOAc (500 mL). The organic phase was washed with water 300 mL, brine 300 mL and concentrated in vacuo. The residue obtained was purified by a chromatography (0-100% EtOAc/petroleum ether) to provide the product 6 as a yellow solid (2.8 g, yield: 50%). LC-MS: [M+H]⁺=563

### 6. Preparation of Compound 7

To a solution of compound 6 (5.6 g, 10 mmol, 1.0 eq.) in DCM (110 mL) was added DIPEA (2.6 g, 20 mmol, 2 eq.), CDI (4.9 g, 30 mmol, 3.0 eq.) at rt. The reaction mixture was stirred for 16 hours. After filtration, the filter cake was washed with petroleum ether (50 mL) and dried to afford the product 7 as an off-white solid (4.7 g, yield: 80%). LC-MS: [M+H]⁺=589

### 7. Preparation of Compound 8

To a solution of compound 7 (5.9 g, 10 mmol, 1.0 eq.) in THF (60 mL) was added tetrabutylammonium fluoride (10 mL, 10 mmol, 1.0 eq.). The reaction mixture was stirred for 3 hours. After diluted with EtOAc (150 mL), washed with H₂O (50 mL) and brine (50 mL). The organic layer was dried over Na₂SO₄ and concentrated in vacuo to give the crude product 8 as an off-white solid (3.16 g, yield: 90%).

### 8. Preparation of Compound 9

To a solution of 7-bromo-6-chloro-8-((2-hydroxyethyl)thio)quinazoline-2,4-diol (3.5 g, 10 mmol, 1.0 eq.) in THF (100 mL) was added PPh₃ (4.5 g, 17 mmol, 1.7 eq.), then DEAD (3.0 g, 17 mmol, 1.7 eq.) at −10˜0° C. The reaction mixture was stirred for 1 hour. After diluted with EtOAc (100 mL), washed with water (100 mL), brine (100 mL). The organic layer was dried over Na₂SO₄ and concentrated in vacuo. The residue obtained was diluted with DCM (100 mL) and stirred for 2 hours. After filtration, the filter cake was washed with DCM (50 mL) and dried to give the product 9 as an off-white solid (1.5 g, yield: 45%). LC-MS: [M+H]+=333.

Compounds of the present disclosure having a substituted, fused 7-membered ring can be prepared in a similar manner.

### EXAMPLES

### Example 100a and 100b: (3S,11S)-8-((3S,5R)-4-acryloyl-3,5-dimethylpiperazin-1-yl)-11-(5-chloro-2,4-difluorophenyl)-3-(pyridin-4-yl)-10-(trifluoromethyl)-3,4-dihydro-2H,6H-[1,4]thiazepino[2,3,4-ij]quinazolin-6-one (example 100a) and (3S)-8-((3S,5R)-4-acryloyl-3,5-dimethylpiperazin-1-yl)-11-(5-chloro-2,4-difluorophenyl)-3-(pyridin-4-yl)-10-(trifluoromethyl)-3,4-dihydro-2H,6H-[1,4]thiazepino[2,3,4-ij]quinazolin-6-one (example 100b)

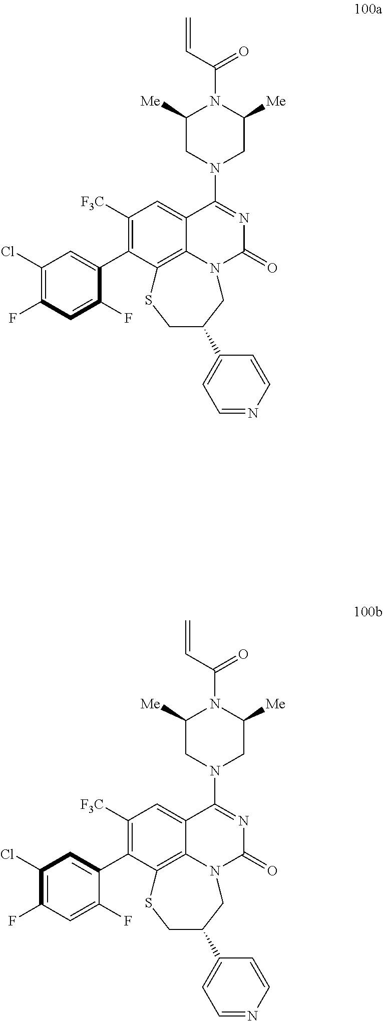

### Step 1: 2,4,7-Trichloro-8-iodo-6-(trifluoromethyl)quinazoline

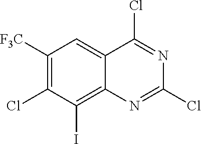 [SMILES: FC(F)(F)c1cc2c(Cl)nc(Cl)nc2c(I)c1Cl]

To a mixture of 7-chloro-8-iodo-6-(trifluoromethyl)quinazoline-2,4(1H,3H)-dione (5.1 mmol) in N,N-diisopropylethylamine (17.9 mmol) was added phosphoryl trichloride (12 mL). The mixture was stirred at 110° C. for 12 hours and the reaction mixture was concentrated under reduced pressure to afford a residue that was taken up in cold water and extracted with ethyl acetate three times. The combined organic layers were washed with brine, dried over anhydrous sodium sulfate, filtered and concentrated under reduced pressure. The resulting crude material was purified by silica gel chromatography (0-15% ethyl acetate in petroleum ether) to afford the title compound in 44% yield as a white solid. MS (ESI) m/z: 427.0 [M+1]+.

### Step 2: tert-Butyl (2S,6R)-4-(2,7-dichloro-8-iodo-6-(trifluoromethyl)quinazolin-4-yl)-2,6-dimethylpiperazine-1-carboxylate

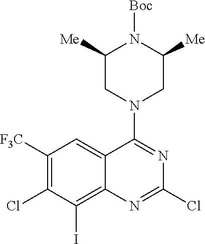 [SMILES: C[C@@H]1CN(c2nc(Cl)nc3c(I)c(Cl)c(C(F)(F)F)cc23)C[C@H](C)N1C(=O)OC(C)(C)C]

To a solution of 2,4,7-trichloro-8-iodo-6-(trifluoromethyl)quinazoline (2.04 mmol) and triethylamine (6.11 mmol) in dichloromethane (10 mL) was added tert-butyl (2S,6R)-2,6-dimethylpiperazine-1-carboxylate (1.83 mmol) at 0° C. The mixture was stirred at room temperature for 1 hour and the volatiles were removed under reduced pressure. The residue was purified by silica gel column chromatography (5-15% ethyl acetate in petroleum ether) to afford the title compound in 82% yield as white solid. MS (ESI) m/z: 605.2 [M+1]+.

### Step 3: tert-Butyl (2S,6R)-4-(7-chloro-8-iodo-2-oxo-6-(trifluoromethyl)-1,2-dihydroquinazolin-4-yl)-2,6-dimethylpiperazine-1-carboxylate

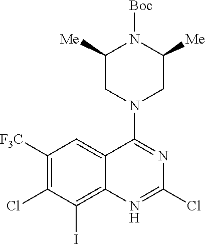 [SMILES: C[C@@H]1CN(c2nc(Cl)nc3c(I)c(Cl)c(C(F)(F)F)cc23)C[C@H](C)N1C(=O)OC(C)(C)C]

To a solution of tert-butyl (2S,6R)-4-(2,7-dichloro-8-iodo-6-(trifluoromethyl)quinazolin-4-yl)-2,6-dimethylpiperazine-1-carboxylate (1.73 mmol) in acetonitrile (50 mL) was added 2-(methylsulfonyl)ethan-1-ol (3.47 mmol), cesium carbonate (3.47 mmol) and 1,4-diazabicyclo[2.2.2]octane (0.17 mmol). The mixture was stirred at 80° C. for 2 hours and the volatiles were removed under reduced pressure to afford a solid that was redissolved in dichloromethane, washed with water, dried over sodium sulfate, filtered and concentrated under reduced pressure. The resulting residue was triturated with a 10:1 mixture of tert-butylmethylether and ethyl acetate (60 mL). The yellow solid was filtered and dried under vacuum to afford the title compound in 88% yield. MS (ESI) m/z: 587.2 [M+1]+.

### Step 4: 2-(Pyridin-4-yl)propane-1,3-diol

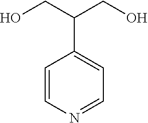 [SMILES: OCC(CO)c1ccncc1]

A mixture of 4-methylpyridine (430 mmol) in 37% formaldehyde (1.72 mol) was stirred at 100° C. for 12 hours. The reaction mixture was concentrated under reduced pressure to afford a residue that was purified by silica gel chromatography (1-10% methanol in ethyl acetate). The title compound was isolated in 88% yield as a colorless oil. 1H NMR (400 MHz, methanol-d4) δ ppm 2.99 (q, J=6.4 Hz, 1H) 3.77-3.91 (m, 4H) 7.29-7.47 (m, 2H) 8.33-8.54 (m, 2H).

### Step 5: 3-Hydroxy-2-(pyridin-4-yl)propyl 4-methylbenzenesulfonate

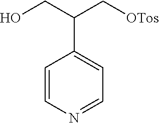

To a 0° C. solution of 2-(pyridin-4-yl)propane-1,3-diol (65.3 mmol) in dichloromethane (100 mL) was added p-toluensulfony lchloride (65.3 mmol), N,N-dimethylaminopyridine (6.53 mmol) and pyridine (65.3 mmol). The mixture was stirred at 25° C. for 3 hours, quenched with water and extracted with ethyl acetate three times. The organic layers were combined and dried over anhydrous sodium sulfate, filtered and concentrated under reduced pressure to afford a residue that was used into the next step without further purification.

### Step 6: S-(3-Hydroxy-2-(pyridin-4-yl)propyl) ethanethioate

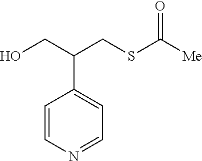 [SMILES: CC(=O)SCC(CO)c1ccncc1]

To a solution of 3-hydroxy-2-(pyridin-4-yl)propyl 4-methylbenzenesulfonate (55.3 mmol) in dimethyl formamide (150 mL) was added potassium thioacetate (81.9 mmol). The mixture was stirred at 50° C. for 2 hours, quenched with water and extracted with ethyl acetate three times. The combined organic layers were dried over anhydrous sodium sulfate, filtered and concentrated under reduced pressure to afford a residue that was purified by silica gel chromatography (50-100% ethyl acetate in petroleum ether). The title compound was isolated in 15% yield as a yellow oil. 1H NMR (400 MHz, CDCl3) δ ppm 2.35 (s, 3H) 2.99 (quin, J=6.4 Hz, 1H) 3.15-3.24 (m, 1H), 3.37 (dd, J=14.0, 7.2 Hz, 1H) 3.79-3.90 (m, 2H) 7.14-7.24 (m, 2H), 8.48-8.59 (m, 2H).

### Step 7: tert-butyl (2S,6R)-4-(7-chloro-8-((3-hydroxy-2-(pyridin-4-yl)propyl)thio)-2-oxo-6-(trifluoromethyl)-1,2-dihydroquinazolin-4-yl)-2,6-dimethylpiperazine-1-carboxylate

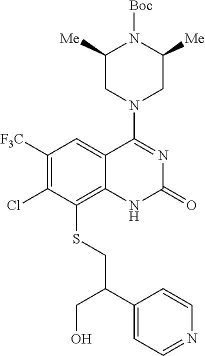 [SMILES: C[C@@H]1CN(c2nc(=O)[nH]c3c(SCC(CO)c4ccncc4)c(Cl)c(C(F)(F)F)cc23)C[C@H](C)N1C(=O)OC(C)(C)C]

To a solution of S-(3-hydroxy-2-(pyridin-4-yl)propyl) ethanethioate (0.95 mmol) and tert-butyl (2S,6R)-4-(7-chloro-8-iodo-2-oxo-6-(trifluoromethyl)-1,2-dihydroquinazolin-4-yl)-2,6-dimethylpiperazine-1-carboxylate (1.42 mmol) in 1,2-ethanediol (2 mL) and isopropanol (2 mL), was added potassium carbonate (2.84 mmol) and cuprous iodide (0.47 mmol). The mixture was stirred at 85° C. for 2 hours, quenched with water and extracted with ethyl acetate three times. The combined organic layers were dried over anhydrous sodium sulfate, filtered and concentrated under reduced pressure to afford a residue that was purified by preparative TLC. The title compound was isolated in 65% yield as a yellow solid. MS (ESI) m/z: 627.9 [M+1]+.

### Step 8: tert-Butyl (2S,6R)-4-((R)-11-chloro-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate (Int-1a) and tert-butyl (2S,6R)-4-((S)-11-chloro-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate (Int-1b)

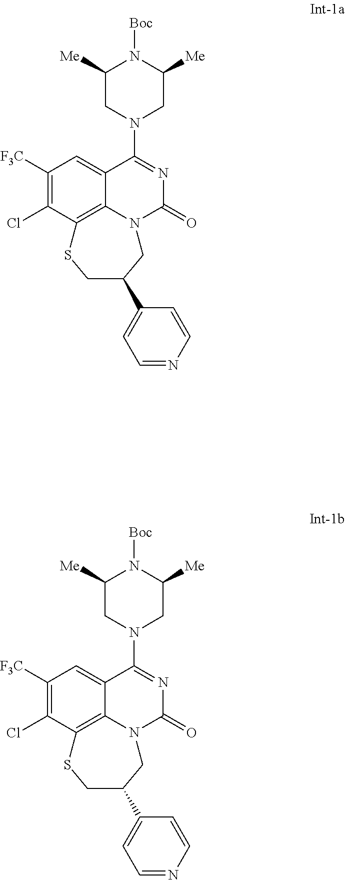

To a 0° C. solution of tert-butyl (2S,6R)-4-(7-chloro-8-((3-hydroxy-2-(pyridin-4-yl)propyl)thio)-2-oxo-6-(trifluoromethyl)-1,2-dihydroquinazolin-4-yl)-2,6-dimethylpiperazine-1-carboxylate (2.11 mmol) in tetrahydrofuran (100 mL) was added triphenylphosphine (10.6 mmol). The mixture was stirred at 0° C. for 30 minutes and diethyl azodicarboxylate (10.6 mmol) was added. After 12 hours, the volatiles were removed under reduced pressure to afford a residue that was purified by silica gel chromatography (0-10% methanol in ethyl acetate). Compounds Int-1a and Int-1b were isolated as a 1:1 mixture of diastereomers in 65% yield. Pure diastereomers Int-1a and Int-1b were obtained by purification of the aforementioned mixture by SFC (Daicel Chiralcel OD 250 mm×30 mm, 10 um using as mobile phase 50% methanol in CO2) and characterized by SFC (Chiracel OD-3, 50×4.6 mm, 3 um using as mobile phase 40% methanol (containing 0.05% diethylamine) in CO2 at 3 mL/min.)
Int-1a: SFC Rt=1.03 min; MS (ESI) m/z: 609.9 [M+1]⁺
Int-1b: SFC Rt=1.40 min; MS (ESI) m/z: 609.9 [M+1]⁺

### Step 9: tert-Butyl (2S,6R)-4-((3S)-11-(5-chloro-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate

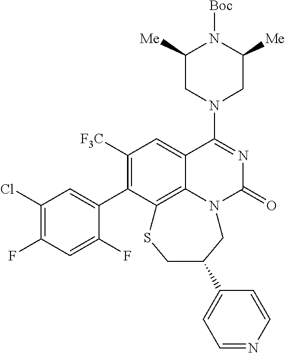 [SMILES: C[C@@H]1CN(c2nc(=O)n3c4c(c(-c5cc(Cl)c(F)cc5F)c(C(F)(F)F)cc24)SC[C@@H](c2ccncc2)C3)C[C@H](C)N1C(=O)OC(C)(C)C]

To a solution of tert-butyl (2S,6R)-4-((S)-11-chloro-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate (0.46 mmol) and (5-chloro-2,4-difluoro-phenyl)boronic acid (1.84 mmol) in dioxane (5 mL) and water (0.1 mL) was added potassium phosphate (1.38 mmol) and Ruphos Pd G4 (0.05 mmol). The mixture was stirred at 80° C. for 30 minutes and the reaction mixture was filtered and concentrated under reduced pressure to afford a residue that was purified by preparative TLC and semi-preparative reverse phase-HPLC. The title compound was isolated in 33% yield as a yellow solid. MS (ESI) m/z: 722.1 [M+1]+.

### Step 10: (3S)-11-(5-chloro-2,4-difluorophenyl)-8-((3S,5R)-3,5-dimethylpiperazin-1-yl)-3-(pyridin-4-yl)-10-(trifluoromethyl)-3,4-dihydro-2H,6H-[1,4]thiazepino[2,3,4-ij]quinazolin-6-one

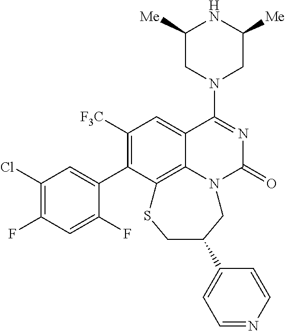 [SMILES: C[C@@H]1CN(c2nc(=O)n3c4c(c(-c5cc(Cl)c(F)cc5F)c(C(F)(F)F)cc24)SC[C@@H](c2ccncc2)C3)C[C@H](C)N1]

To a solution of tert-butyl (2S,6R)-4-((3S)-11-(5-chloro-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate (0.17 mmol) in dichloromethane (3 mL) was added trifluoroacetic acid (1 mL) at 0° C. The mixture was stirred at 0° C. for 30 minutes followed by evaporation of volatiles under reduced pressure. The title compound was isolated in 87% yield as a yellow oil and used in the next step without further purification. MS (ESI) m/z: 622.0 [M+1]+.

### Step 11: (3S,11S)-8-((3S,5R)-4-acryloyl-3,5-dimethylpiperazin-1-yl)-11-(5-chloro-2,4-difluorophenyl)-3-(pyridin-4-yl)-10-(trifluoromethyl)-3,4-dihydro-2H,6H-[1,4]thiazepino[2,3,4-ij]quinazolin-6-one (example 100a) and (3S)-8-((3S,5R)-4-acryloyl-3,5-dimethylpiperazin-1-yl)-11-(5-chloro-2,4-difluorophenyl)-3-(pyridin-4-yl)-10-(trifluoromethyl)-3,4-dihydro-2H,6H-[1,4]thiazepino[2,3,4-ij]quinazolin-6-one (example 100b)

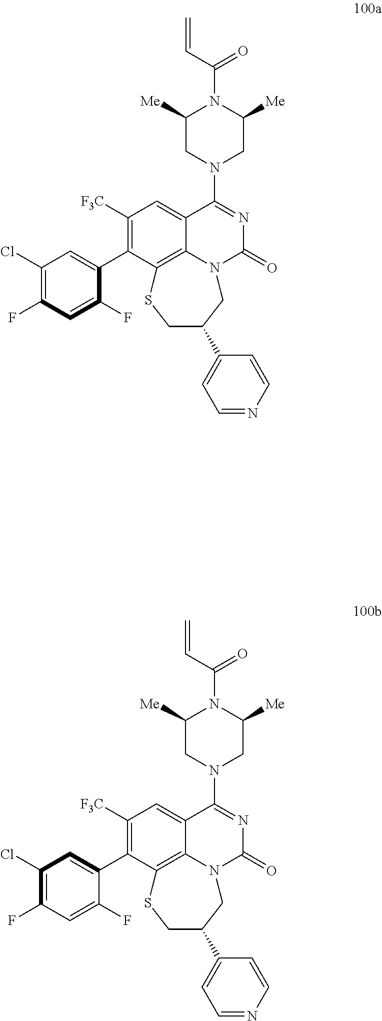

To a 0° C. solution of (3S)-11-(5-chloro-2,4-difluorophenyl)-8-((3S,5R)-3,5-dimethylpiperazin-1-yl)-3-(pyridin-4-yl)-10-(trifluoromethyl)-3,4-dihydro-2H,6H-[1,4]thiazepino[2,3,4-ij]quinazolin-6-one (0.16 mmol) in dichloromethane (3 mL) was added triethylamine (0.05 mmol) and prop-2-enoyl chloride (0.32 mmol). The mixture was stirred at 0° C. for 30 minutes and the volatiles were removed under reduced pressure to afford a residue that was purified by preparative TLC. Compounds 1a and 1b were isolated as a 1:1 mixture of diastereomers in 54% yield. Pure diastereomers 1a and 1b were obtained by purification of the aforementioned mixture by SFC (Daicel Chiralpak AD 250 mm×30 mm, 10 um, using as mobile phase 50% isopropanol in CO2) and characterized by SFC (Chiralpak AD-3, 50×4.6 mm, 3 um. Using as mobile phase: 5-40% isopropanol (containing 0.05% diethylamine) in CO2 at 3 mL/min.)

Compound 100a: SFC Rt=1.79 min; MS (ESI) m/z: 676.0 [M+1]+. ¹H NMR (400 MHz, CDCl3) δ ppm 1.51-1.59 (m, 6H) 3.15 (br d, J=13.6 Hz, 1H) 3.41 (td, J=12.8, 4.8 Hz, 2H) 3.70-3.87 (m, 2H) 4.12-4.27 (m, 2H) 4.53-4.91 (m, 4H) 5.80 (dd, J=10.4, 2.0 Hz, 1H) 6.43 (dd, J=16.8, 2.0 Hz, 1H) 6.64 (dd, J=16.4, 10.4 Hz, 1H) 7.06 (t, J=8.8 Hz, 1H) 7.28-7.34 (m, 3H) 8.14 (s, 1H) 8.49-8.67 (m, 2H).

Compound 100b: SFC Rt=3.30 min; MS (ESI) m/z: 676.0 [M+1]+. 1H NMR (400 MHz, CDCl3) δ ppm 1.53 (br d, J=6.8 Hz, 6H) 3.06-3.27 (m, 1H) 3.41 (ddd, J=17.6, 13.2, 4.4 Hz, 2H) 3.61-3.83 (m, 2H) 4.14-4.23 (m, 2H) 4.53-4.90 (m, 4H) 5.80 (dd, J=10.4, 2.0 Hz, 1H) 6.43 (dd, J=16.8, 1.6 Hz, 1H) 6.64 (dd, J=16.8, 10.4 Hz, 1H) 7.10 (t, J=8.8 Hz, 1H) 7.23-7.27 (m, 1H) 7.30 (br d, J=5.2 Hz, 2H) 8.15 (s, 1H) 8.59 (d, J=6.0 Hz, 2H).

### Example 101a and 101b: (3R)-8-((3S,5R)-4-acryloyl-3,5-dimethylpiperazin-1-yl)-11-(5-chloro-2,4-difluorophenyl)-3-(pyridin-4-yl)-10-(trifluoromethyl)-3,4-dihydro-2H,6H-[1,4]thiazepino[2,3,4-ij]quinazolin-6-one (101a) and (3R,11S)-8-((3S,5R)-4-acryloyl-3,5-dimethylpiperazin-1-yl)-11-(5-chloro-2,4-difluorophenyl)-3-((S)-3,4-dihydropyridin-4-yl)-10-(trifluoromethyl)-3,4-dihydro-2H,6H-[1,4]thiazepino[2,3,4-ij]quinazolin-6-one (101b)

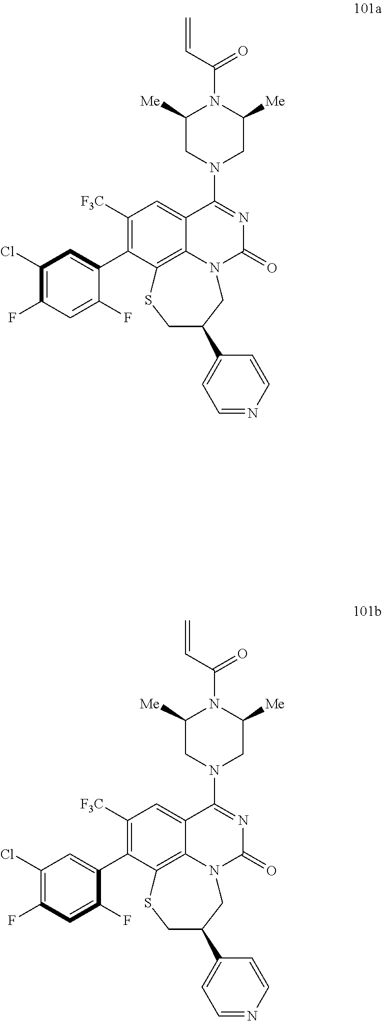

### Step 1: tert-Butyl (2S,6R)-4-((3R)-11-(5-chloro-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate

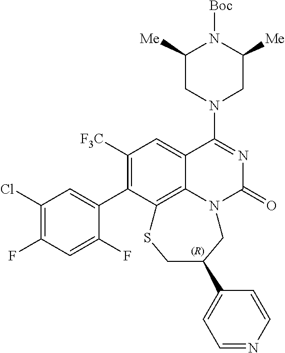

Potassium phosphate (1.18 mmol) and Ruphos Pd G4 (0.04 mmol) were added to a solution of tert-butyl (2S,6R)-4-((R)-11-chloro-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate (0.39 mmol) and (5-chloro-2,4-difluoro-phenyl)boronic acid (1.57 mmol) in dioxane (5 mL) and water (0.1 mL). The mixture was stirred at 80° C. for 30 minutes, cooled down to room temperature and the insoluble materials were filtered. Evaporation of volatiles under reduced pressure afforded a residue that was purified by preparative TLC. The title compound was isolated in 610% yield as a yellow solid. MS (ESI) m/z: 722.1 [M+1]+.

### Step 2: (3R)-11-(5-Chloro-2,4-difluorophenyl)-8-((3S,5R)-3,5-dimethylpiperazin-1-yl)-3-(pyridin-4-yl)-10-(trifluoromethyl)-3,4-dihydro-2H,6H-[1,4]thiazepino[2,3,4-ij]quinazolin-6-one

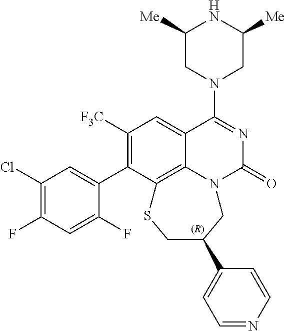

Trifluoroacetic acid (1 mL) was added to a 0° C. solution of tert-butyl (2S,6R)-4-((3R)-11-(5-chloro-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate (0.25 mmol) in dichloromethane (3 mL). The reaction was stirred at 0° C. for 30 minutes and the volatiles were removed under reduced pressure to afford the title compound in 97% yield as a yellow oil. MS (ESI) m/z: 622.0 [M+1]+.

### Step 3: (3R)-8-((3S,5R)-4-acryloyl-3,5-dimethylpiperazin-1-yl)-11-(5-chloro-2,4-difluorophenyl)-3-(pyridin-4-yl)-10-(trifluoromethyl)-3,4-dihydro-2H,6H-[1,4]thiazepino[2,3,4-ij]quinazolin-6-one (101a) and (3R,11S)-8-((3S,5R)-4-acryloyl-3,5-dimethylpiperazin-1-yl)-11-(5-chloro-2,4-difluorophenyl)-3-((S)-3,4-dihydropyridin-4-yl)-10-(trifluoromethyl)-3,4-dihydro-2H,6H-[1,4]thiazepino[2,3,4-ij]quinazolin-6-one (101b)

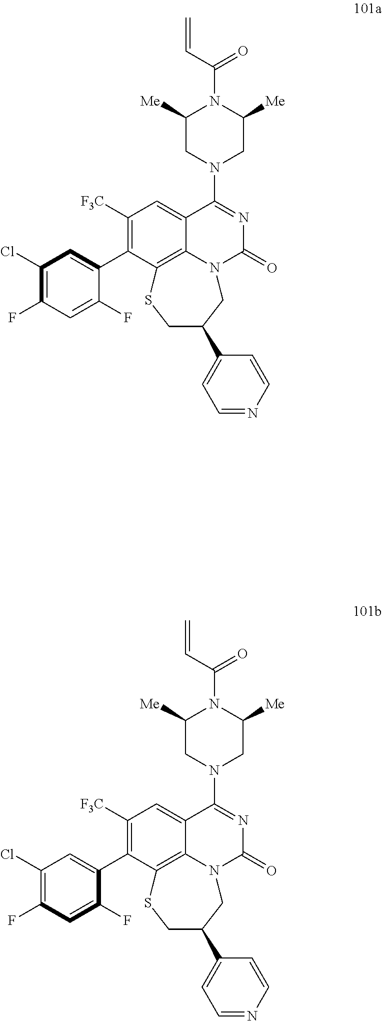

To a 0° C. solution of (3R)-11-(5-Chloro-2,4-difluorophenyl)-8-((3S,5R)-3,5-dimethylpiperazin-1-yl)-3-(pyridin-4-yl)-10-(trifluoromethyl)-3,4-dihydro-2H,6H-[1,4]thiazepino[2,3,4-ij]quinazolin-6-one (0.16 mmol) in dichloromethane (3 mL) was added triethylamine (0.05 mmol) and prop-2-enoyl chloride (0.32 mmol). The mixture was stirred at 0° C. for 30 minutes and the volatiles were removed under reduced pressure to afford a residue that was purified by preparative TLC. Compounds 101a and 101b were isolated as a 1:1 mixture of diastereomers in 94% yield.

Pure diastereomers 101a and 101b were obtained by purification of the aforementioned mixture by SFC (column: Daicel Chiralpak IG 250 mm×30 mm, 10 um using as mobile phase 55% isopropanol/acetonitrile (4:1) in CO2). Each individual diastereomer was analyzed by SFC (Chiralpak AD-3, 50×4.6 mm, 3 um, using as mobile phase 40% isopropanol (containing 0.05% diethylamine) in CO2 at 3 mL/min).

Compound 101a: SFC Rt=1.25 min; MS (ESI) m/z: 676.0 [M+1]+. 1H NMR (400 MHz, CDCl3) δ ppm=1.50-1.61 (m, 6H) 3.16 (br d, J=12.8 Hz, 1H) 3.41 (td, J=12.4, 4.8 Hz, 2H) 3.67-3.95 (m, 2H) 4.12-4.25 (m, 2H) 4.42-5.05 (m, 4H) 5.72-5.90 (m, 1H) 6.43 (dd, J=16.8, 1.6 Hz, 1H) 6.56-6.72 (m, 1H) 7.06 (t, J=8.8 Hz, 1H) 7.31 (t, J=7.6 Hz, 1H) 7.38 (d, J=6.0 Hz, 2H) 8.14 (s, 1H) 8.62 (d, J=6.0 Hz, 2H).

Compound 101b: SFC Rt=2.23 min; MS (ESI) m/z: 676.0 [M+1]+. 1H NMR (400 MHz, CDCl3) δ ppm=1.48-1.61 (m, 6H) 2.99-3.24 (m, 1H) 3.41 (ddd, J=17.6, 13.2, 4.6 Hz, 2H) 3.57-3.89 (m, 2H) 4.19 (br dd, J=13.2, 5.6 Hz, 2H) 4.46-5.00 (m, 4H) 5.80 (dd, J=10.4, 1.6 Hz, 1H) 6.43 (dd, J=16.8, 1.6 Hz, 1H) 6.64 (dd, J=16.8, 10.4 Hz, 1H) 7.10 (t, J=8.8 Hz, 1H) 7.25 (s, 1H) 7.31 (br d, J=5.6 Hz, 2H) 8.15 (s, 1H) 8.52-8.67 (m, 2H).

### Example 102: (3S)-8-((3S,5R)-4-acryloyl-3,5-dimethylpiperazin-1-yl)-11-(5-bromo-2,4-difluorophenyl)-3-(pyridin-4-yl)-10-(trifluoromethyl)-3,4-dihydro-2H,6H-[1,4]thiazepino[2,3,4-ij]quinazolin-6-one

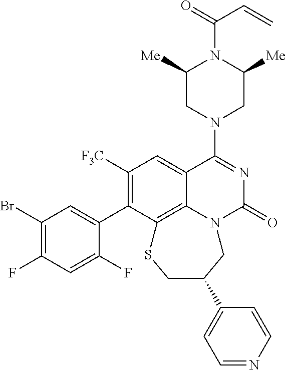

### Step 1: tert-butyl (2S,6R)-4-((3S)-11-(5-amino-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate

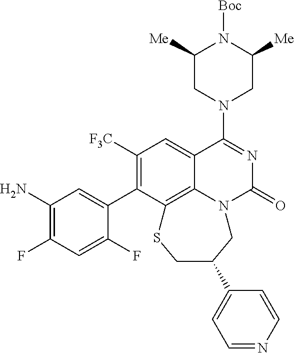

The title compound was prepared analogously to Example 100, step 9, where (5-chloro-2,4-difluoro-phenyl)boronic acid was replaced with 2,4-difluoro-5-(4,4,5,5-tetramethyl-1,3,2-dioxaborolan-2-yl)aniline. The title compound was isolated in 78% yield as a yellow solid. MS (ESI) m/z: 703.2 [M+1]+.

### Step 2: tert-butyl (2S,6R)-4-((3S)-11-(5-bromo-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate

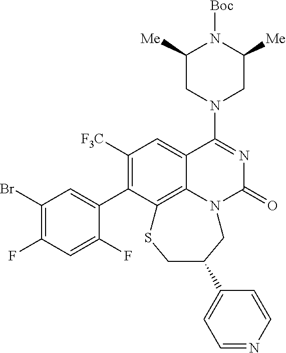

To a solution of copper bromide (0.14 mmol) and tert-butyl nitrite (0.17 mmol) in acetonitrile (1 mL) was added tert-butyl (2R,6S)-4-[(12S)-8-(5-amino-2,4-difluoro-phenyl)-2-oxo-12-(4-pyridyl)-7-(trifluoromethyl)-10-thia-1,3-diazatricyclo[7.4.1.05,14]tetradeca-3,5,7,9(14)-tetraen-4-yl]-2,6-dimethyl-piperazine-1-carboxylate (0.11 mmol) in acetonitrile (1 mL) and the reaction was stirred at 60° C. for 30 minutes. The reaction mixture was filtered and concentrated under reduced pressure to afford a residue that was purified by preparative TLC followed by semi-preparative reverse phase-HPLC. The title compound was isolated in 40% yield as a yellow solid. MS (ESI) m/z: 768.2 [M+1]+.

### Step 3: (3S)-11-(5-Bromo-2,4-difluorophenyl)-8-((3S,5R)-3,5-dimethylpiperazin-1-yl)-3-(pyridin-4-yl)-10-(trifluoromethyl)-3,4-dihydro-2H,6H-[1,4]thiazepino[2,3,4-ij]quinazolin-6-one

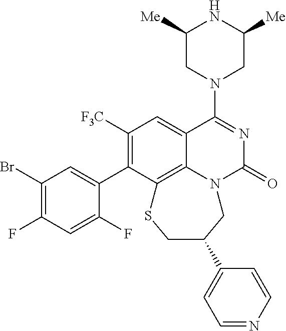 [SMILES: C[C@@H]1CN(c2nc(=O)n3c4c(c(-c5cc(Br)c(F)cc5F)c(C(F)(F)F)cc24)SC[C@@H](c2ccncc2)C3)C[C@H](C)N1]

The title compound was prepared analogously to Example 100, step 10, where tert-butyl (2S,6R)-4-((3S)-11-(5-chloro-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((3S)-11-(5-bromo-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate. The title compound was isolated in 99% yield as a yellow oil. MS (ESI) m/z: 668.1 [M+1]+.

### Step 4: (3S)-8-((3S,5R)-4-Acryloyl-3,5-dimethylpiperazin-1-yl)-11-(5-bromo-2,4-difluorophenyl)-3-(pyridin-4-yl)-10-(trifluoromethyl)-3,4-dihydro-2H,6H-[1,4]thiazepino[2,3,4-ij]quinazolin-6-one

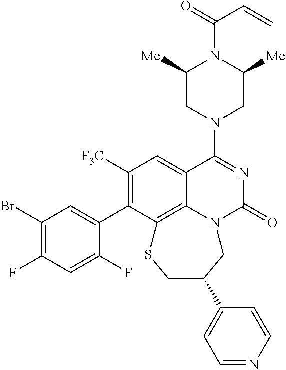

The title compound was prepared analogously to Example 100, step 11, where (3S)-11-(5-chloro-2,4-difluorophenyl)-8-((3S,5R)-3,5-dimethylpiperazin-1-yl)-3-(pyridin-4-yl)-10-(trifluoromethyl)-3,4-dihydro-2H,6H-[1,4]thiazepino[2,3,4-ij]quinazolin-6-one was replaced with (3S)-11-(5-Bromo-2,4-difluorophenyl)-8-((3S,5R)-3,5-dimethylpiperazin-1-yl)-3-(pyridin-4-yl)-10-(trifluoromethyl)-3,4-dihydro-2H,6H-[1,4]thiazepino[2,3,4-ij]quinazolin-6-one. The title compound was isolated in 97% yield as a yellow oil. MS (ESI) m/z: 722.1 [M+1]+. 1H NMR (400 MHz, CDCl3) δ ppm 1.54 (br d, J=6.8 Hz, 6H) 3.04-3.25 (m, 1H) 3.34-3.46 (m, 2H) 3.57-3.89 (m, 2H) 4.19 (br dd, J=13.6, 6.8 Hz, 2H) 4.50-4.93 (m, 4H) 5.80 (dd, J=10.4, 1.6 Hz, 1H) 6.38-6.49 (m, 1H) 6.56-6.71 (m, 1H) 6.99-7.15 (m, 1H) 7.31 (br dd, J=4.4, 2.0 Hz, 2H) 7.43 (dt, J=14.4, 7.2 Hz, 1H) 8.14 (d, J=3.6 Hz, 1H) 8.51-8.68 (m, 2H).

### Example 104a and 104b: (3S,11R)-8-((3S,5R)-4-acryloyl-3,5-dimethylpiperazin-1-yl)-11-(5-chloro-2,4-difluorophenyl)-3-(pyridin-3-yl)-10-(trifluoromethyl)-3,4-dihydro-2H,6H-[1,4]thiazepino[2,3,4-ij]quinazolin-6-one (example 104a) and (3S,11S)-8-((3S,5R)-4-acryloyl-3,5-dimethylpiperazin-1-yl)-11-(5-chloro-2,4-difluorophenyl)-3-(pyridin-3-yl)-10-(trifluoromethyl)-3,4-dihydro-2H,6H-[1,4]thiazepino[2,3,4-ij]quinazolin-6-one (example 104b)

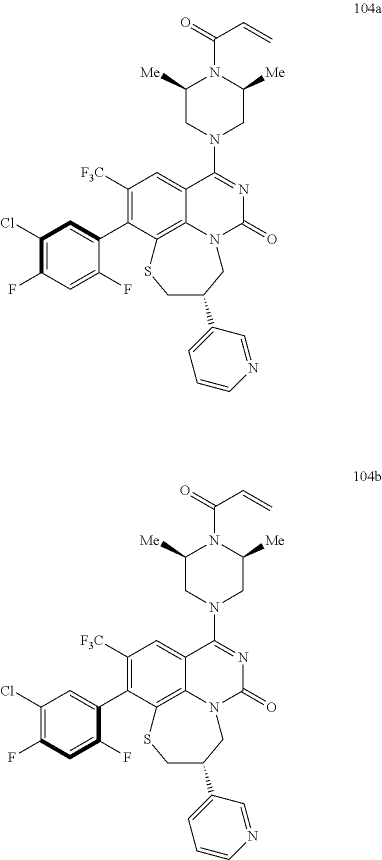

### Step 1: 2-(pyridin-3-yl)prop-2-en-1-ol

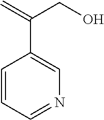

A mixture of pyridin-3-ylboronic acid (41 mmol), 2-bromoprop-2-en-1-ol (49 mmol), potassium phosphate (122 mmol) and XPhosPd G2 (1.0 mmol) in THF (50 mL) and water (50 mL) was stirred at 60° C. for 12 hours. The reaction mixture was diluted with water, extracted with ethyl acetate three times and the combined organic layers were dried over anhydrous sodium sulfate, filtered and concentrated under reduced pressure to afford a residue that was purified by silica gel chromatography (35-80% ethyl acetate in hexanes). The title compound was isolated in 53% yield as colorless oil. MS (ESI) m/z: 136.1 [M+H]+.

### Step 2: 2-(pyridin-3-yl)propane-1,3-diol

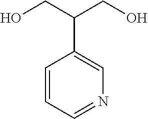 [SMILES: OCC(CO)c1cccnc1]

A 10M THF solution of borane dimethyl sulfide complex (8.6 mL) was added over a 0° C. solution of 2-(pyridin-3-yl)prop-2-en-1-ol (22 mmol) in THF (30 mL). After 5 minutes, a 1M aqueous solution of NaOH (6.5 mL) was added dropwise, followed by a 35% aqueous solution of hydrogen peroxide (86 mmol). After 2 hours, methanol (50 mL) was added and the resulting reaction was stirred at 70° C. for 12 hours. The reaction mixture was diluted with water, extracted with ethyl acetate three times and the combined organic layers were dried over anhydrous sodium sulfate, filtered and concentrated under reduced pressure to afford a residue that was purified by silica gel chromatography (0-20% methanol in ethyl acetate). The title compound was isolated in 73% yield as colorless oil. 1H NMR (400 MHz, CDCl3) δ 8.58-8.48 (m, 2H), 7.68-7.59 (m, 1H), 7.32-7.28 (m, 1H), 4.10-3.96 (m, 4H), 3.16-3.07 (m, 1H).

### Step 3: 3-((tert-butyldiphenylsilyl)oxy)-2-(pyridin-3-yl)propan-1-ol

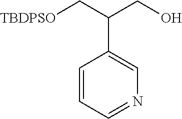

2-(Pyridin-3-yl)propane-1,3-diol (13.0 mmol) was added to a 0° C. suspension of NaH (13.1 mmol) in THF (20 mL). The reaction was stirred at room temperature for 30 minutes, cooled down to 0° C. and a solution of TBDPSCl (10.4 mmol) in THF (20 mL) was added. After 30 minutes, the reaction was stopped by the addition of a saturated aqueous solution of ammonium chloride, extracted with ethyl acetate, washed with brine, dried over sodium sulfate, filtered and concentrated under reduced pressure to afford a residue that was purified by silica gel chromatography (20-50% ethyl acetate in hexanes). The title compound was isolated in 47% yield as a colorless oil. 1H NMR (400 MHz, CDCl3) δ 8.49 (d, J=4.4 Hz, 1H), 8.45 (s, 1H), 7.61 (dd, J=7.6, 11.2 Hz, 4H), 7.54 (d, J=8.0 Hz, 1H), 7.49-7.33 (m, 6H), 7.21 (dd, J=4.8, 7.6 Hz, 1H), 4.11-4.05 (m, 1H), 3.99-3.89 (m, 3H), 3.08 (quin, J=6.0 Hz, 1H), 2.14 (s, 1H), 1.05 (s, 9H).

### Step 4: S-(3-((tert-butyldiphenylsilyl)oxy)-2-(pyridin-3-yl)propyl) ethanethioate

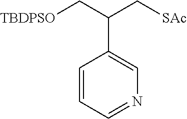

A solution of triphenylphosphine (8.20 mmol) and DIAD (8.17 mmol) in THF (50 mL) was stirred at 0° C. for 30 minutes. 3-((tert-butyldiphenylsilyl)oxy)-2-(pyridin-3-yl)propan-1-ol (4.09 mmol) and ethanethioic S-acid (9.81 mmol) were added and the mixture was stirred at room temperature for one hour. Evaporation of volatiles under reduced pressure afforded a residue that was purified by silica gel chromatography (10-25% ethyl acetate in hexanes). The title compound was isolated in 98% yield as a colorless oil. MS (ESI) m/z: 450.1 [M+H]+.

### Step 5: tert-butyl (2S,6R)-4-(8-((3-((tert-butyldiphenylsilyl)oxy)-2-(pyridin-3-yl)propyl)thio)-7-chloro-2-oxo-6-(trifluoromethyl)-1,2-dihydroquinazolin-4-yl)-2,6-dimethylpiperazine-1-carboxylate

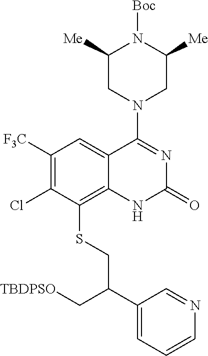

The title compound was prepared analogously to Example 100, step 7 where S-(3-hydroxy-2-(pyridin-4-yl)propyl) ethanethioate was replaced with S-(3-((tert-butyldiphenylsilyl)oxy)-2-(pyridin-3-yl)propyl) ethanethioate. The title compound was isolated in 97% yield as a brown solid. MS (ESI) m/z: 866.3 [M+H]+.

### Step 6: tert-butyl (2S,6R)-4-(7-chloro-8-((3-hydroxy-2-(pyridin-3-yl)propyl)thio)-2-oxo-6-(trifluoromethyl)-1,2-dihydroquinazolin-4-yl)-2,6-dimethylpiperazine-1-carboxylate

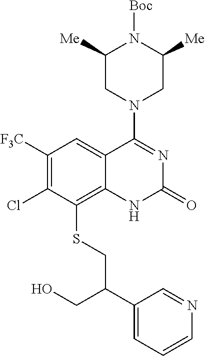 [SMILES: C[C@@H]1CN(c2nc(=O)[nH]c3c(SCC(CO)c4cccnc4)c(Cl)c(C(F)(F)F)cc23)C[C@H](C)N1C(=O)OC(C)(C)C]

A 1M solution of TBAF in THF (4.4 mL) was added over a solution of tert-butyl (2S,6R)-4-(8-((3-((tert-butyldiphenylsilyl)oxy)-2-(pyridin-3-yl)propyl)thio)-7-chloro-2-oxo-6-(trifluoromethyl)-1,2-dihydroquinazolin-4-yl)-2,6-dimethylpiperazine-1-carboxylate (3.69 mmol) in THF (50 mL). After 2 hours the reaction mixture was diluted with water, extracted with ethyl acetate three times and the combined organic layers were dried over anhydrous sodium sulfate, filtered and concentrated under reduced pressure to afford a residue that was purified by silica gel chromatography (20% methanol in ethyl acetate). The title compound was isolated in 80% yield as brown solid. MS (ESI) m/z: 628.2 [M+H]+.

### Step 7: tert-butyl (2S,6R)-4-((S)-11-chloro-6-oxo-3-(pyridin-3-yl)-10-(trifluoromethyl)-3,4-dihydro-2H,6H-[1,4]thiazepino[2,3,4-ij]quinazolin-8-yl)-2,6-dimethylpiperazine-1-carboxylate and tert-butyl (2S,6R)-4-((R)-11-chloro-6-oxo-3-(pyridin-3-yl)-10-(trifluoromethyl)-3,4-dihydro-2H,6H-[1,4]thiazepino[2,3,4-ij]quinazolin-8-yl)-2,6-dimethylpiperazine-1-carboxylate

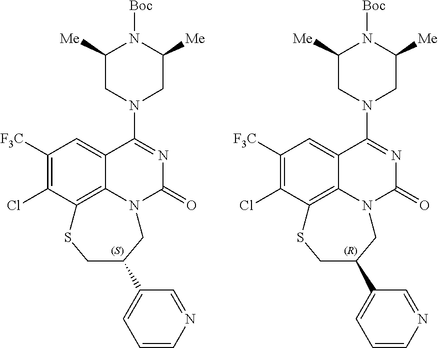

The title compounds were prepared analogously to Example 100, step 8 where tert-butyl (2S,6R)-4-(7-chloro-8-((3-hydroxy-2-(pyridin-4-yl)propyl)thio)-2-oxo-6-(trifluoromethyl)-1,2-dihydroquinazolin-4-yl)-2,6-dimethylpiperazine-1-carboxylate was replaced with tert-butyl (2S,6R)-4-(7-chloro-8-((3-hydroxy-2-(pyridin-3-yl)propyl)thio)-2-oxo-6-(trifluoromethyl)-1,2-dihydroquinazolin-4-yl)-2,6-dimethylpiperazine-1-carboxylate. The title compounds were isolated in 98% yield as a 1:1 mixture of diastereomers. Each individual diastereomer was isolated by purification of the aforementioned mixture by SFC (Chiracel Chiralpak AD 250×30 mm, 10 um using 40% methanol in CO2 as mobile phase). Each individual diastereomer was characterized by analytical SFC (Chiralpak AD-3, 50×4.6 mm, 3 um using as mobile phase 5-40% ethanol (containing 0.05% of diethylamine) in CO2 at 3 mL/min.)

tert-butyl (2S,6R)-4-((S)-11-chloro-6-oxo-3-(pyridin-3-yl)-10-(trifluoromethyl)-3,4-dihydro-2H,6H-[1,4]thiazepino[2,3,4-ij]quinazolin-8-yl)-2,6-dimethylpiperazine-1-carboxylate: Rt=1.94 min. MS (ESI) m/z: 610.1 [M+H]+. 1H NMR (400 MHz, DMSO-d6) δ 9.38-8.16 (m, 2H), 8.01 (s, 1H), 7.86 (s, 1H), 7.63-7.25 (m, 1H), 4.85-4.40 (m, 2H), 4.32-4.16 (m, 2H), 4.04-3.95 (m, 3H), 3.68 (br s, 2H), 3.17 (dt, J=4.0, 12.4 Hz, 2H), 1.44 (s, 9H), 1.42-1.26 (m, 6H).

tert-butyl (2S,6R)-4-((R)-11-chloro-6-oxo-3-(pyridin-3-yl)-10-(trifluoromethyl)-3,4-dihydro-2H,6H-[1,4]thiazepino[2,3,4-ij]quinazolin-8-yl)-2,6-dimethylpiperazine-1-carboxylate: Rt=2.42 min. MS (ESI) m/z: 610.1 [M+H]+. 1H NMR (400 MHz, DMSO-d6) δ 8.63 (d, J=7.6 Hz, 1H), 8.01 (s, 1H), 7.87 (d, J=3.2 Hz, 1H), 7.64-7.29 (m, 1H), 5.01-4.42 (m, 2H), 4.29-4.16 (m, 2H), 4.05-3.99 (m, 3H), 3.94-3.59 (m, 2H), 3.17 (dt, J=4.0, 12.4 Hz, 2H), 1.44 (s, 9H), 1.41-1.26 (m, 6H).

### Step 8: tert-butyl (2S,6R)-4-((3S)-11-(5-chloro-2,4-difluorophenyl)-6-oxo-3-(pyridin-3-yl)-10-(trifluoromethyl)-3,4-dihydro-2H,6H-[1,4]thiazepino[2,3,4-ij]quinazolin-8-yl)-2,6-dimethylpiperazine-1-carboxylate

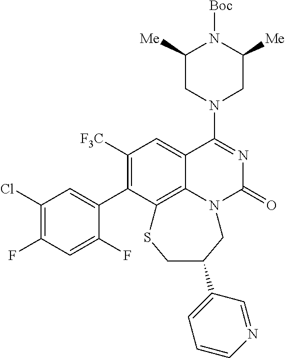 [SMILES: C[C@@H]1CN(c2nc(=O)n3c4c(c(-c5cc(Cl)c(F)cc5F)c(C(F)(F)F)cc24)SC[C@@H](c2cccnc2)C3)C[C@H](C)N1C(=O)OC(C)(C)C]

The title compound was prepared analogously to Example 100, step 9, where tert-butyl (2S,6R)-4-((S)-11-chloro-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((S)-11-chloro-6-oxo-3-(pyridin-3-yl)-10-(trifluoromethyl)-3,4-dihydro-2H,6H-[1,4]thiazepino[2,3,4-ij]quinazolin-8-yl)-2,6-dimethylpiperazine-1-carboxylate. The title compound was isolated in 52% yield as a white solid. MS (ESI) m/z: 722.1 [M+H]+.

### Step 9: (3S)-11-(5-chloro-2,4-difluorophenyl)-8-((3S,5R)-3,5-dimethylpiperazin-1-yl)-3-(pyridin-3-yl)-10-(trifluoromethyl)-3,4-dihydro-2H,6H-[1,4]thiazepino[2,3,4-ij]quinazolin-6-one

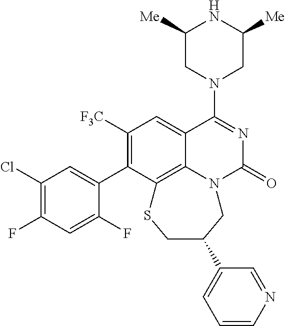 [SMILES: C[C@@H]1CN(c2nc(=O)n3c4c(c(-c5cc(Cl)c(F)cc5F)c(C(F)(F)F)cc24)SC[C@@H](c2cccnc2)C3)C[C@H](C)N1]

The title compound was prepared analogously to Example 100, step 10 where tert-butyl (2S,6R)-4-((3S)-11-(5-chloro-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((3S)-11-(5-chloro-2,4-difluorophenyl)-6-oxo-3-(pyridin-3-yl)-10-(trifluoromethyl)-3,4-dihydro-2H,6H-[1,4]thiazepino[2,3,4-ij]quinazolin-8-yl)-2,6-dimethylpiperazine-1-carboxylate.

The title compound was isolated in 99% yield as a yellow oil. MS (ESI) m/z: 622.1 [M+H]+.

### Step 10: (3S,11R)-8-((3S,5R)-4-acryloyl-3,5-dimethylpiperazin-1-yl)-11-(5-chloro-2,4-difluorophenyl)-3-(pyridin-3-yl)-10-(trifluoromethyl)-3,4-dihydro-2H,6H-[1,4]thiazepino[2,3,4-ij]quinazolin-6-one (example 104a) and (3S,11S)-8-((3S,5R)-4-acryloyl-3,5-dimethylpiperazin-1-yl)-11-(5-chloro-2,4-difluorophenyl)-3-(pyridin-3-yl)-10-(trifluoromethyl)-3,4-dihydro-2H,6H-[1,4]thiazepino[2,3,4-ij]quinazolin-6-one (Example 104b)

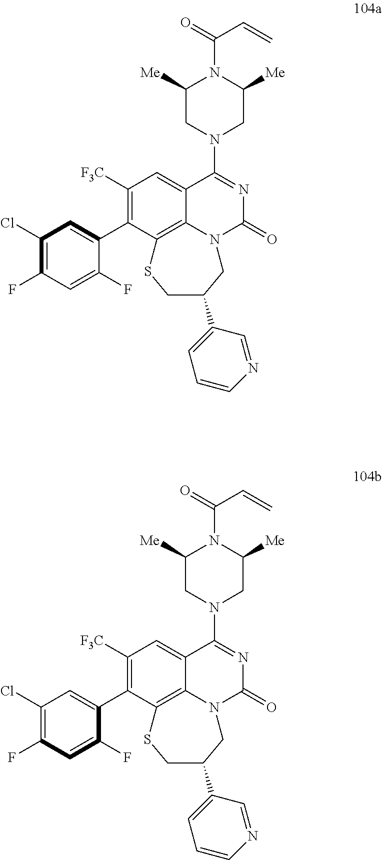

The title compounds were prepared analogously to Example 100, step 11 where (3S)-11-(5-chloro-2,4-difluorophenyl)-8-((3S,5R)-3,5-dimethylpiperazin-1-yl)-3-(pyridin-4-yl)-10-(trifluoromethyl)-3,4-dihydro-2H,6H-[1,4]thiazepino[2,3,4-ij]quinazolin-6-one was replaced with (3S)-11-(5-chloro-2,4-difluorophenyl)-8-((3S,5R)-3,5-dimethylpiperazin-1-yl)-3-(pyridin-3-yl)-10-(trifluoromethyl)-3,4-dihydro-2H,6H-[1,4]thiazepino[2,3,4-ij]quinazolin-6-one. The title compounds were isolated in 66% yield as a white solid and as a 1:1 mixture of diastereomers. Each individual diastereomer was obtained by purification of the aforementioned mixture by SFC (Chiracel Chiralpak IC 250×30 mm, 10 um using 65% methanol in CO2 as mobile phase). Each individual diastereomer was characterized by analytical SFC (Chiralpak IC-3, 50×4.6 mm, 3 um using as mobile phase 60% methanol (containing 0.05% of diethylamine) in CO2 at 3 mL/min.

(3S,11R)-8-((3S,5R)-4-acryloyl-3,5-dimethylpiperazin-1-yl)-11-(5-chloro-2,4-difluorophenyl)-3-(pyridin-3-yl)-10-(trifluoromethyl)-3,4-dihydro-2H,6H-[1,4]thiazepino[2,3,4-ij]quinazolin-6-one (Example 104a): Rt=2.98 min. MS (ESI) m/z: 676.0 [M+H]+. 1H NMR (400 MHz, CDCl3) δ 8.87-8.39 (m, 2H), 8.15 (s, 1H), 7.74 (d, J=6.8 Hz, 1H), 7.40-7.28 (m, 2H), 7.10 (t, J=8.8 Hz, 1H), 6.64 (dd, J=10.4, 16.4 Hz, 1H), 6.43 (dd, J=2.0, 16.4 Hz, 1H), 5.83-5.76 (m, 1H), 5.11-4.48 (m, 4H), 4.19 (d, J=13.2 Hz, 2H), 3.89-3.58 (m, 2H), 3.46-3.36 (m, 2H), 3.27-3.00 (m, 1H), 1.61-1.53 (m, 6H).

(3S,11S)-8-((3S,5R)-4-acryloyl-3,5-dimethylpiperazin-1-yl)-11-(5-chloro-2,4-difluorophenyl)-3-(pyridin-3-yl)-10-(trifluoromethyl)-3,4-dihydro-2H,6H-[1,4]thiazepino[2,3,4-ij]quinazolin-6-one (Example 104b): Rt=3.62 min. MS (ESI) m/z: 676.0 [M+H]+. 1H NMR (400 MHz, CDCl3) δ 8.91-8.30 (m, 2H), 8.14 (s, 1H), 7.75 (d, J=7.6 Hz, 1H), 7.32 (t, J=7.6 Hz, 2H), 7.10-7.02 (m, 1H), 6.70-6.58 (m, 1H), 6.47-6.39 (m, 1H), 5.83-5.76 (m, 1H), 4.91-4.54 (m, 4H), 4.23-4.15 (m, 2H), 3.91-3.72 (m, 2H), 3.40 (dt, J=4.4, 13.2 Hz, 2H), 3.22-3.03 (m, 1H), 1.60 (d, J=6.8 Hz, 3H), 1.54 (d, J=6.8 Hz, 3H).

### Example 106: (R)-8-((3S,5R)-4-acryloyl-3,5-dimethylpiperazin-1-yl)-11-(4-fluorophenyl)-3-(pyridin-2-yl)-10-(trifluoromethyl)-3,4-dihydro-2H,6H-[1,4]thiazepino[2,3,4-ij]quinazolin-6-one

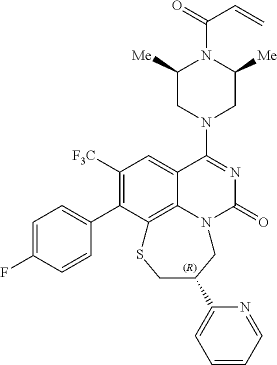

Step 1: tert-butyl (2S,6R)-4-((S)-11-chloro-6-oxo-3-(pyridin-2-yl)-10-(trifluoromethyl)-3,4-dihydro-2H,6H-[1,4]thiazepino[2,3,4-ij]quinazolin-8-yl)-2,6-dimethylpiperazine-1-carboxylate and tert-butyl (2S,6R)-4-((R)-11-chloro-6-oxo-3-(pyridin-2-yl)-10-(trifluoromethyl)-3,4-dihydro-2H,6H-[1,4]thiazepino[2,3,4-ij]quinazolin-8-yl)-2,6-dimethylpiperazine-1-carboxylate

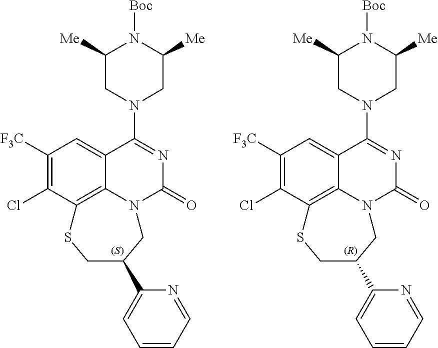

The title compounds were prepared analogously to Example 100, where 4-methylpyridine was replaced with 2-methylpyridine in step 4. The title compounds were isolated as a 1:1 mixture of diastereomers. Each individual diastereomer was obtained by purification of the aforementioned mixture by SFC (Chiracel Chiralpak AS, 250×50 mm, 10 um using 40% methanol in CO2 as mobile phase) and characterized by analytical SFC (Chiralpak IC-3, 50×4.6 mm, 3 um using as mobile phase 5-40% methanol (containing 0.05% of diethylamine) in CO2 at 3 mL/min.)

tert-butyl (2S,6R)-4-((S)-11-chloro-6-oxo-3-(pyridin-2-yl)-10-(trifluoromethyl)-3,4-dihydro-2H,6H-[1,4]thiazepino[2,3,4-ij]quinazolin-8-yl)-2,6-dimethylpiperazine-1-carboxylate: Rt=1.12 minutes. MS (ESI) m/z: 610.2 [M+H]+.

tert-butyl (2S,6R)-4-((R)-11-chloro-6-oxo-3-(pyridin-2-yl)-10-(trifluoromethyl)-3,4-dihydro-2H,6H-[1,4]thiazepino[2,3,4-ij]quinazolin-8-yl)-2,6-dimethylpiperazine-1-carboxylate: Rt=1.39 minutes. MS (ESI) m/z: 610.2 [M+H]+.

### Step 2: tert-butyl (2S,6R)-4-((R)-11-(4-fluorophenyl)-6-oxo-3-(pyridin-2-yl)-10-(trifluoromethyl)-3,4-dihydro-2H,6H-[1,4]thiazepino[2,3,4-ij]quinazolin-8-yl)-2,6-dimethylpiperazine-1-carboxylate

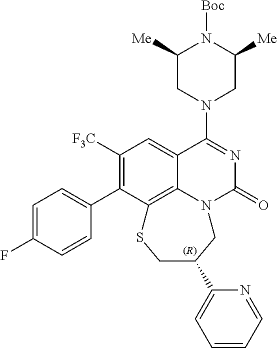

The title compound was prepared analogously to Example 100, step 9, where tert-butyl (2S,6R)-4-((S)-11-chloro-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((R)-11-chloro-6-oxo-3-(pyridin-2-yl)-10-(trifluoromethyl)-3,4-dihydro-2H,6H-[1,4]thiazepino[2,3,4-ij]quinazolin-8-yl)-2,6-dimethylpiperazine-1-carboxylate. The title compound was isolated in 91% yield as a yellow solid. MS (ESI) m/z: 670.2 [M+H]+.

### Step 3: (R)-8-((3S,5R)-3,5-dimethylpiperazin-1-yl)-11-(4-fluorophenyl)-3-(pyridin-2-yl)-10-(trifluoromethyl)-3,4-dihydro-2H,6H-[1,4]thiazepino[2,3,4-ij]quinazolin-6-one

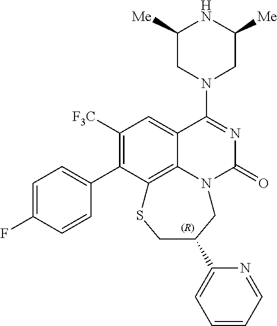

The title compound was prepared analogously to Example 100, step 10 where tert-butyl (2S,6R)-4-((3S)-11-(5-chloro-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((R)-11-(4-fluorophenyl)-6-oxo-3-(pyridin-2-yl)-10-(trifluoromethyl)-3,4-dihydro-2H,6H-[1,4]thiazepino[2,3,4-ij]quinazolin-8-yl)-2,6-dimethylpiperazine-1-carboxylate. The title compound was isolated in 95% yield as a yellow solid. MS (ESI) m/z: 570.2 [M+H]+

### Step 4: (R)-8-((3S,5R)-4-acryloyl-3,5-dimethylpiperazin-1-yl)-11-(4-fluorophenyl)-3-(pyridin-2-yl)-10-(trifluoromethyl)-3,4-dihydro-2H,6H-[1,4]thiazepino[2,3,4-ij]quinazolin-6-one

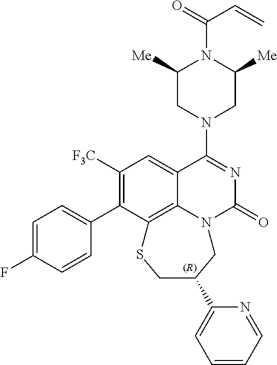

The title compound was prepared analogously to Example 100, step 11 where tert-butyl (2S,6R)-4-((3S)-11-(5-chloro-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with (R)-8-((3S,5R)-3,5-dimethylpiperazin-1-yl)-11-(4-fluorophenyl)-3-(pyridin-2-yl)-10-(trifluoromethyl)-3,4-dihydro-2H,6H-[1,4]thiazepino[2,3,4-ij]quinazolin-6-one. The title compound was isolated in 33% yield as a yellow solid. MS (ESI) m/z: 624.3 [M+H]+ 0.1H NMR (400 MHz, CDCl3-d) δ, 8.46 (br d, J=4.4 Hz, 1H), 8.03 (s, 1H), 7.59 (dt, J=1.6, 7.6 Hz, 1H), 7.23 (d, J=7.6 Hz, 1H), 7.17-7.06 (m, 5H), 6.57 (dd, J=10.4, 16.8 Hz, 1H), 6.35 (dd, J=1.6, 16.8 Hz, 1H), 5.76-5.65 (m, 1H), 4.92 (br d, J=4.4 Hz, 2H), 4.76-4.39 (m, 2H), 4.15-4.07 (m, 2H), 3.92-3.75 (m, 1H), 3.59-3.40 (m, 2H), 3.32-3.23 (m, 2H), 1.46 (br d, J=6.8 Hz, 6H).

### Examples 107a and Example 107b: (3R,11S)-8-((3S,5R)-4-acryloyl-3,5-dimethylpiperazin-1-yl)-11-(5-bromo-2,4-difluorophenyl)-3-(pyridin-2-yl)-10-(trifluoromethyl)-3,4-dihydro-2H,6H-[1,4]thiazepino[2,3,4-ij]quinazolin-6-one and (3R,11R)-8-((3S,5R)-4-acryloyl-3,5-dimethylpiperazin-1-yl)-11-(5-bromo-2,4-difluorophenyl)-3-(pyridin-2-yl)-10-(trifluoromethyl)-3,4-dihydro-2H,6H-[1,4]thiazepino[2,3,4-ij]quinazolin-6-one

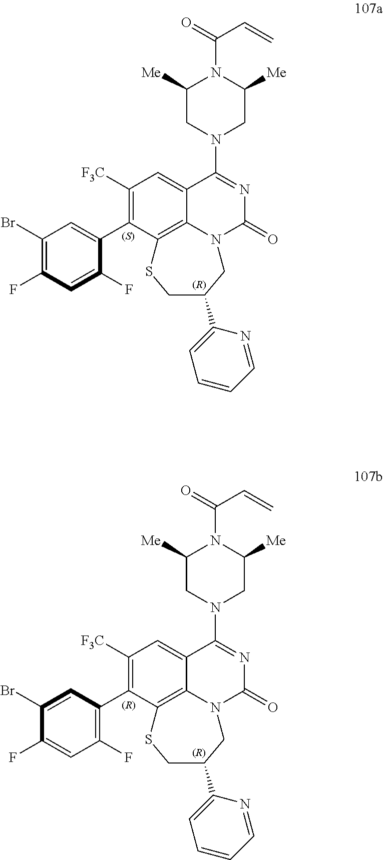

The title compounds were prepared analogously to Example 102, where tert-butyl (2S,6R)-4-((S)-11-chloro-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((R)-11-chloro-6-oxo-3-(pyridin-2-yl)-10-(trifluoromethyl)-3,4-dihydro-2H,6H-[1,4]thiazepino[2,3,4-ij]quinazolin-8-yl)-2,6-dimethylpiperazine-1-carboxylate. The title compounds were obtained as a 1:1 mixture of diastereomers. Each individual diastereomer was obtained by purification of the aforementioned mixture by SFC (Chiracel Chiralpak AD, 250×30 mm, 10 um using 45% isopropanol (containing 0.1% of ammonium hydroxide) in CO2 as mobile phase) and characterized by analytical SFC (Chiralpak AD, 50×4.6 mm, 3 um using as mobile phase 40% isopropanol (containing 0.05% of diethylamine) in CO2 at 3 mL/min.)

Example 107a: Rt=0.46 minutes. MS (ESI) m/z: 721.9 [M+H]+. ¹H NMR ¹H NMR (400 MHz, CDCl₃-d) δ, 8.57 (br d, J=4.4 Hz, 1H), 8.12 (s, 1H), 7.73-7.64 (m, 1H), 7.42 (t, J=7.2 Hz, 1H), 7.36-7.30 (m, 1H), 7.23-7.16 (m, 1H), 7.06-6.97 (m, 1H), 6.64 (dd, J=10.4, 16.8 Hz, 1H), 6.43 (dd, J=2.0, 16.8 Hz, 1H), 5.79 (dd, J=2.0, 10.4 Hz, 1H), 5.08-4.93 (m, 2H), 4.84-4.49 (m, 2H), 4.22-4.13 (m, 2H), 4.01-3.91 (m, 1H), 3.71-3.52 (m, 2H), 3.41-3.32 (m, 2H), 1.55 (br d, J=7.2 Hz, 6H).

Example 107b: Rt=0.83 minutes. MS (ESI) m/z: 721.9 [M+H]+. 1H NMR (400 MHz, CDCl3-d) δ, 8.47 (br s, 1H), 8.11-8.01 (m, 1H), 7.67-7.55 (m, 1H), 7.35 (br t, J=7.2 Hz, 1H), 7.26 (br d, J=7.2 Hz, 1H), 7.15-7.10 (m, 1H), 6.95 (t, J=8.8 Hz, 1H), 6.56 (dd, J=10.4, 16.8 Hz, 1H), 6.35 (dd, J=2.0, 16.8 Hz, 1H), 5.74-5.68 (m, 1H), 4.92 (br d, J=4.4 Hz, 2H), 4.74-4.23 (m, 2H), 4.09 (br d, J=12.8 Hz, 2H), 3.95-3.76 (m, 1H), 3.70-3.35 (m, 2H), 3.34-3.21 (m, 2H), 1.49 (br s, 6H).

### Example 108a and 108b: (3S,11R)-8-((3S,5R)-4-acryloyl-3,5-dimethylpiperazin-1-yl)-11-(5-bromo-2,4-difluorophenyl)-3-(pyridin-3-yl)-10-(trifluoromethyl)-3,4-dihydro-2H,6H-[1,4]thiazepino[2,3,4-ij]quinazolin-6-one and (3S,11S)-8-((3S,5R)-4-acryloyl-3,5-dimethylpiperazin-1-yl)-11-(5-bromo-2,4-difluorophenyl)-3-(pyridin-3-yl)-10-(trifluoromethyl)-3,4-dihydro-2H,6H-[1,4]thiazepino[2,3,4-ij]quinazolin-6-one

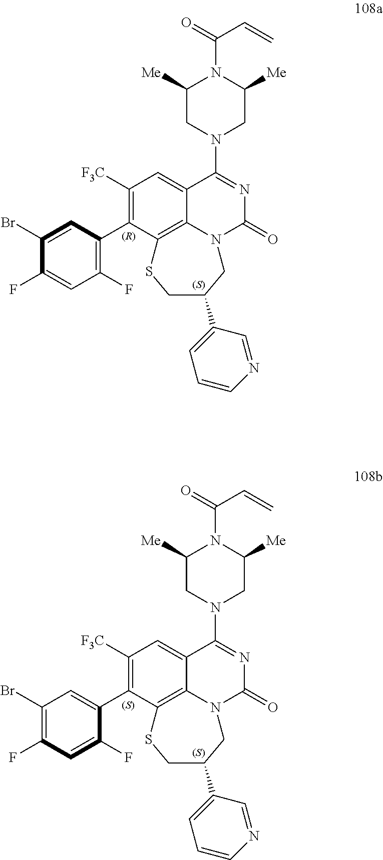

The title compounds were prepared analogously to Example 102, where tert-butyl (2S,6R)-4-((S)-11-chloro-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((S)-11-chloro-6-oxo-3-(pyridin-3-yl)-10-(trifluoromethyl)-3,4-dihydro-2H,6H-[1,4]thiazepino[2,3,4-ij]quinazolin-8-yl)-2,6-dimethylpiperazine-1-carboxylate. The title compounds were obtained as a 1:1 mixture of diastereomers. Each individual diastereomer was obtained by purification of the aforementioned mixture by SFC (Phenomenex cellulose-2, 250×30 mm, 10 um using 70% methanol in CO2 as mobile phase) and characterized by analytical SFC (Unichiral OZ-5H, 50×4.6 mm, 3 um using as mobile phase 60% methanol (containing 0.05% of diethylamine) in CO2 at 3 mL/min).

Example 108a: Rt=1.89 minutes. MS (ESI) m/z: 720.0 [M+H]+. 1H NMR (400 MHz, CDCl3) δ 9.15-8.23 (m, 1H), 8.15 (s, 1H), 7.75 (d, J=6.8 Hz, 1H), 7.42 (t, J=7.2 Hz, 1H), 7.37-7.27 (m, 2H), 7.08 (t, J=8.4 Hz, 1H), 6.70-6.58 (m, 1H), 6.43 (dd, J=2.0, 16.4 Hz, 1H), 5.80 (dd, J=2.0, 10.4 Hz, 1H), 5.09-4.31 (m, 4H), 4.19 (d, J=12.8 Hz, 2H), 3.97-3.54 (m, 2H), 3.46-3.34 (m, 2H), 3.29-2.97 (m, 1H), 1.60 (d, J=6.8 Hz, 3H), 1.53 (d, J=6.8 Hz, 3H).

Example 108b: Rt=3.03 minutes. MS (ESI) m/z: 720.0 [M+H]+. ¹H NMR (400 MHz, CDCl₃) δ 9.10-8.29 (m, 1H), 8.14 (s, 1H), 7.84-7.73 (m, 1H), 7.46 (t, J=7.0 Hz, 1H), 7.38-7.27 (m, 2H), 7.04 (t, J=8.4 Hz, 1H), 6.64 (dd, J=10.4, 16.4 Hz, 1H), 6.48-6.38 (m, 1H), 5.80 (dd, J=1.6, 10.4 Hz, 1H), 5.07-4.42 (m, 4H), 4.19 (dd, J 5.2, 13.6 Hz, 2H), 3.98-3.71 (m, 2H), 3.45-3.36 (m, 2H), 3.17-3.09 (m, 1H), 1.59 (d, J=6.8 Hz, 3H), 1.54 (d, J=6.8 Hz, 3H).

### Example 109:(3S)-8-((3S,5R)-4-acryloyl-3,5-dimethylpiperazin-1-yl)-11-(5-chloro-2,4-difluorophenyl)-3-(5-fluoropyridin-3-yl)-10-(trifluoromethyl)-3,4-dihydro-2H,6H-[1,4]thiazepino[2,3,4-ij]quinazolin-6-one

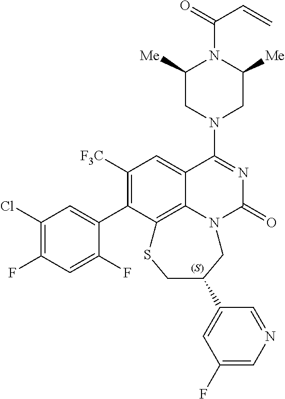

### Step 1: tert-butyl (2S,6R)-4-((R)-11-chloro-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate and tert-butyl (2S,6R)-4-((S)-11-chloro-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate

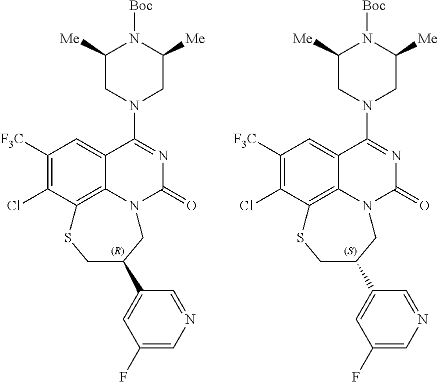

The title compounds were prepared analogously to Examples 104a and 104b, where pyridin-3-ylboronic acid was replaced with (5-fluoropyridin-3-yl)boronic acid in step 1. The title compounds were obtained as a 1:1 mixture of diastereomers and each individual diastereomer was isolated by purification of the aforementioned mixture by SFC (Chiracel Chiralpak IC 250×30 mm, 10 um using 65% ethanol in CO2 as mobile phase) and characterized by analytical SFC (Chiralpak IC-3, 50×4.6 mm, 3 um using as mobile phase 60% ethanol (containing 0.05% of diethylamine) in CO2 at 3 mL/min).

tert-butyl (2S,6R)-4-((R)-11-chloro-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate: Rt=1.58 minutes. MS (ESI) m/z: 628.1 [M+H]+.

tert-butyl (2S,6R)-4-((S)-11-chloro-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate: Rt=2.87 minutes. MS (ESI) m/z: 628.1 [M+H]+.

### Step 2: tert-butyl (2S,6R)-4-((3S)-11-(5-chloro-2,4-difluorophenyl)-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate

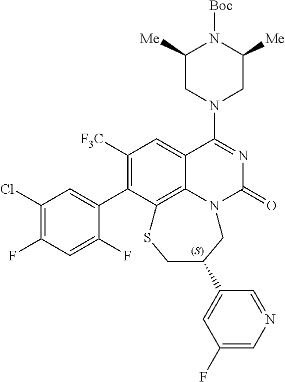

The title compound was prepared analogously to Example 100, step 9, where tert-butyl (2S,6R)-4-((S)-11-chloro-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((S)-11-chloro-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate. The title compound was isolated in 85% yield as a yellow solid. MS (ESI) m/z: 740.0 [M+H]+.

### Step 3: (3S)-11-(5-chloro-2,4-difluorophenyl)-8-((3S,5R)-3,5-dimethylpiperazin-1-yl)-3-(5-fluoropyridin-3-yl)-10-(trifluoromethyl)-3,4-dihydro-2H,6H-[1,4]thiazepino[2,3,4-ij]quinazolin-6-one

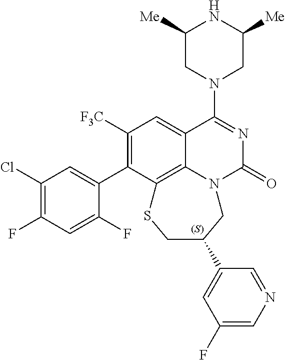

The title compound was prepared analogously to Example 100, step 10 where tert-butyl (2S,6R)-4-((3S)-11-(5-chloro-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((3S)-11-(5-chloro-2,4-difluorophenyl)-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate. The title compound was isolated in 99% yield as a yellow solid. MS (ESI) m/z: 640.0 [M+H]+.

### Step 4: (3S)-8-((3S,5R)-4-acryloyl-3,5-dimethylpiperazin-1-yl)-11-(5-chloro-2,4-difluorophenyl)-3-(5-fluoropyridin-3-yl)-10-(trifluoromethyl)-3,4-dihydro-2H,6H-[1,4]thiazepino[2,3,4-ij]quinazolin-6-one

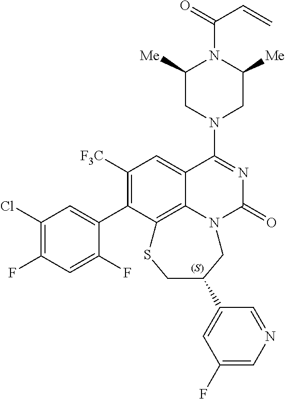

The title compound was prepared analogously to Example 100, step 11, where (3S)-11-(5-chloro-2,4-difluorophenyl)-8-((3S,5R)-3,5-dimethylpiperazin-1-yl)-3-(pyridin-4-yl)-10-(trifluoromethyl)-3,4-dihydro-2H,6H-[1,4]thiazepino[2,3,4-ij]quinazolin-6-one was replaced with (3S)-11-(5-chloro-2,4-difluorophenyl)-8-((3S,5R)-3,5-dimethylpiperazin-1-yl)-3-(5-fluoropyridin-3-yl)-10-(trifluoromethyl)-3,4-dihydro-2H,6H-[1,4]thiazepino[2,3,4-ij]quinazolin-6-one. The title compound was isolated in 21% yield as a white solid. MS (ESI) m/z: 694.4 [M+H]+. 1H NMR (400 MHz, CDCl3) δ 8.48 (s, 1H), 8.43 (d, J=2.4 Hz, 1H), 8.14 (s, 1H), 7.48 (d, J=9.2 Hz, 1H), 7.36-7.27 (m, 1H), 7.14-7.03 (m, 1H), 6.68-6.60 (m, 1H), 6.47-6.40 (m, 1H), 5.80 (d, J=11.6 Hz, 1H), 4.97-4.54 (m, 4H), 4.19 (dd, J=5.2, 12.0 Hz, 2H), 3.96-3.67 (m, 2H), 3.41 (t, J=4.4, 12.4 Hz, 2H), 3.20-3.04 (m, 1H), 1.59 (d, J=6.8 Hz, 3H), 1.54 (d, J=6.8 Hz, 3H).

### Example 112a and Example 112b: (3S,11S)-8-((3S,5R)-4-acryloyl-3,5-dimethylpiperazin-1-yl)-11-(5-chloro-2,4-difluorophenyl)-3-(3-fluoropyridin-4-yl)-10-(trifluoromethyl)-3,4-dihydro-2H,6H-[1,4]thiazepino[2,3,4-ij]quinazolin-6-one (12a) and (3S,11R)-8-((3S,5R)-4-acryloyl-3,5-dimethylpiperazin-1-yl)-11-(5-chloro-2,4-difluorophenyl)-3-(3-fluoropyridin-4-yl)-10-(trifluoromethyl)-3,4-dihydro-2H,6H-[1,4]thiazepino[2,3,4-ij]quinazolin-6-one (12b)

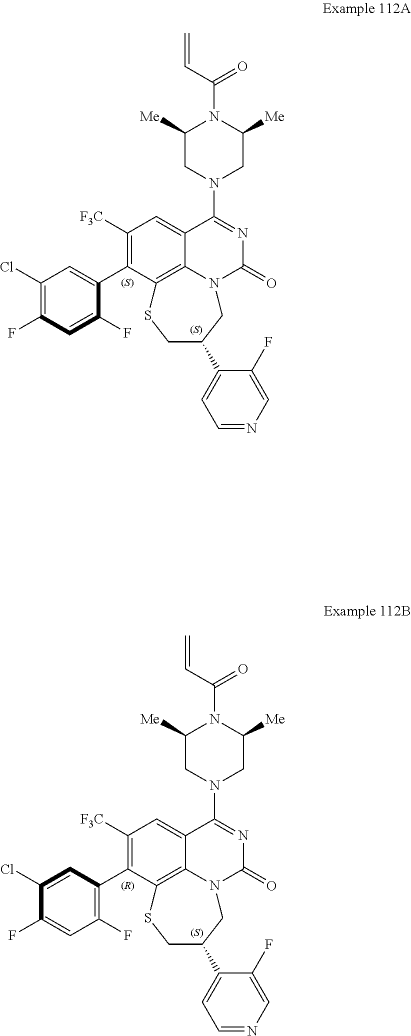

### Step 1: tert-butyl (2S,6R)-4-((S)-11-chloro-3-(3-fluoropyridin-4-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate and tert-butyl (2S,6R)-4-((R)-11-chloro-3-(3-fluoropyridin-4-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate

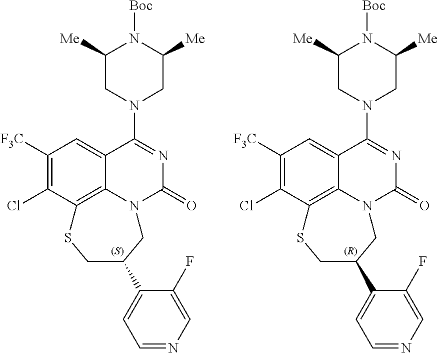

The title compounds were prepared analogously to Example 100, where 4-methylpyridine was replaced with 3-fluoro-4-methyl-pyridine in step 4. These two compounds were obtained as a 1:1 mixture of diastereomers. Each individual diastereomer was isolated by purification of the aforementioned mixture by SFC (Chiracel Chiralpak IC 250×30 mm, 10 um using 60% methanol (containing 0.1% of ammonium hydroxide) in CO2 as mobile phase) and characterized by analytical SFC ((S,S)Whelk-O1, 50×4.6 mm, 1.8 um, with 50% methanol/acetonitrile (4:1) (containing 0.05% of diethylamine) in CO2 as mobile phase at 3 mL/min).

tert-butyl (2S,6R)-4-((S)-11-chloro-3-(3-fluoropyridin-4-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate: Rt=1.41 minutes. MS (ESI) m/z: 628.4 [M+H]+

tert-butyl (2S,6R)-4-((R)-11-chloro-3-(3-fluoropyridin-4-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate: Rt=1.81 minutes MS (ESI) m/z: 628.4 [M+H]+

### Step 2: tert-butyl (2S,6R)-4-((3S)-11-(5-chloro-2,4-difluorophenyl)-3-(3-fluoropyridin-4-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate

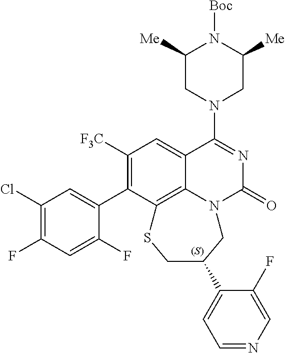

The title compound was prepared analogously to Example 100, step 9, where tert-butyl (2S,6R)-4-((S)-11-chloro-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((S)-11-chloro-3-(3-fluoropyridin-4-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate. The title compound was isolated in 44% yield as a yellow solid. MS (ESI) m/z: 740.5 [M+H]+.

### Step 2: (3S)-11-(5-chloro-2,4-difluorophenyl)-8-((3S,5R)-3,5-dimethylpiperazin-1-yl)-3-(3-fluoropyridin-4-yl)-10-(trifluoromethyl)-3,4-dihydro-2H,6H-[1,4]thiazepino[2,3,4-ij]quinazolin-6-one

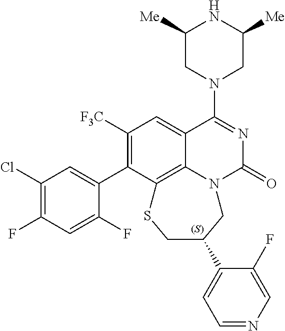

The title compound was prepared analogously to Example 100, step 10 where tert-butyl (2S,6R)-4-((3S)-11-(5-chloro-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((3S)-11-(5-chloro-2,4-difluorophenyl)-3-(3-fluoropyridin-4-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate. The title compound was isolated in 98% yield as a yellow solid. MS (ESI) m/z: 640.5 [M+H]+.

### Step 3: (3S,11S)-8-((3S,5R)-4-acryloyl-3,5-dimethylpiperazin-1-yl)-11-(5-chloro-2,4-difluorophenyl)-3-(3-fluoropyridin-4-yl)-10-(trifluoromethyl)-3,4-dihydro-2H,6H-[1,4]thiazepino[2,3,4-ij]quinazolin-6-one (example 112a) and (3S,11R)-8-((3S,5R)-4-acryloyl-3,5-dimethylpiperazin-1-yl)-11-(5-chloro-2,4-difluorophenyl)-3-(3-fluoropyridin-4-yl)-10-(trifluoromethyl)-3,4-dihydro-2H,6H-[1,4]thiazepino[2,3,4-ij]quinazolin-6-one (Example 112b)

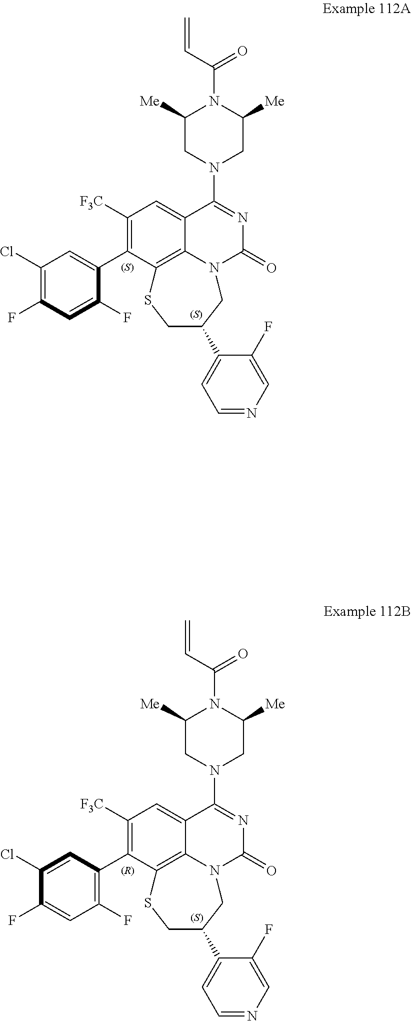

The title compounds were prepared analogously to Example 100, step 11 where (3S)-11-(5-chloro-2,4-difluorophenyl)-8-((3S,5R)-3,5-dimethylpiperazin-1-yl)-3-(pyridin-4-yl)-10-(trifluoromethyl)-3,4-dihydro-2H,6H-[1,4]thiazepino[2,3,4-ij]quinazolin-6-one was replaced with (3S)-11-(5-chloro-2,4-difluorophenyl)-8-((3S,5R)-3,5-dimethylpiperazin-1-yl)-3-(3-fluoropyridin-4-yl)-10-(trifluoromethyl)-3,4-dihydro-2H,6H-[1,4]thiazepino[2,3,4-ij]quinazolin-6-one. These two compounds were isolated in 51% yield as a 1:1 mixture of diastereomers. Each individual diastereomer was isolated by purification of the mixture by SFC (Chiracel OD 250×30 mm, 10 um using 50% methanol in CO2 as mobile phase) and characterized by analytical SFC (Chiralpak IC-3, 50×4.6 mm, 3 um using as mobile phase 60% methanol/acetonitrile (containing 0.05% of diethylamine) in CO2 at 3 mL/min).

(3S,11S)-8-((3S,5R)-4-acryloyl-3,5-dimethylpiperazin-1-yl)-11-(5-chloro-2,4-difluorophenyl)-3-(3-fluoropyridin-4-yl)-10-(trifluoromethyl)-3,4-dihydro-2H,6H-[1,4]thiazepino[2,3,4-ij]quinazolin-6-one (112a) (Example 112a): Rt=0.92 minutes. MS (ESI) m/z: 694.1 [M+H]+. 1H NMR (400 MHz, CDCl3) δ ppm 1.54 (d, J=6.8 Hz, 3H) 1.59 (d, J=6.8 Hz, 3H) 3.02-3.23 (m, 1H) 3.42 (td, J=12.4, 4.4 Hz, 2H) 3.65-3.86 (m, 1H) 4.11-4.24 (m, 3H) 4.54-5.01 (m, 4H) 5.74-5.84 (m, 1H) 6.38-6.48 (m, 1H) 6.58-6.70 (m, 1H) 7.05 (t, J=8.8 Hz, 1H) 7.30 (t, J=7.6 Hz, 2H) 8.14 (s, 1H) 8.32-8.61 (m, 2H).

(3S,11R)-8-((3S,5R)-4-acryloyl-3,5-dimethylpiperazin-1-yl)-11-(5-chloro-2,4-difluorophenyl)-3-(3-fluoropyridin-4-yl)-10-(trifluoromethyl)-3,4-dihydro-2H,6H-[1,4]thiazepino[2,3,4-ij]quinazolin-6-one (example 112b): Rt=1.37 minutes. MS (ESI) m/z: 694.1 [M+H]+. 1H NMR (400 MHz, CDCl3) δ ppm 1.53 (d, J=6.4 Hz, 3H) 1.59 (br s, 3H) 2.97-3.27 (m, 1H) 3.36-3.46 (m, 2H) 3.54-3.83 (m, 1H) 4.05-4.26 (m, 3H) 4.49-5.05 (m, 4H) 5.80 (dd, J=10.4, 2.0 Hz, 1H) 6.43 (dd, J=16.8, 2.0 Hz, 1H) 6.64 (dd, J=16.8, 10.4 Hz, 1H) 7.09 (t, J=8.8 Hz, 1H) 7.25 (br s, 1H) 7.29 (br s, 1H) 8.14 (s, 1H), 8.44 (d, 4.8 Hz), 8.47 (s, 1H).

### Example 113: (S)-8-((3S,5R)-4-acryloyl-3,5-dimethylpiperazin-1-yl)-11-(4-fluorophenyl)-3-(pyridin-4-yl)-10-(trifluoromethyl)-3,4-dihydro-2H,6H-[1,4]thiazepino[2,3,4-ij]quinazolin-6-one

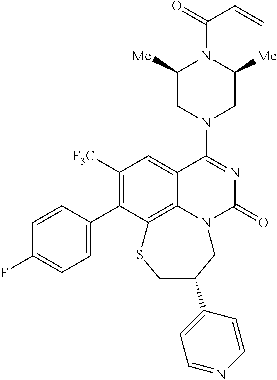

### Step 1: tert-butyl (2S,6R)-4-((S)-11-(4-fluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate

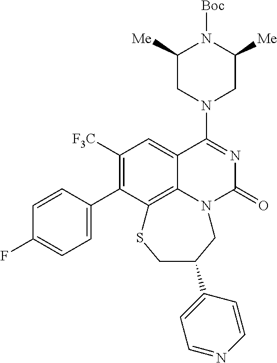

The title compound was prepared analogously to Example 100, step 9, where (5-chloro-2,4-difluoro-phenyl)boronic acid was replaced with (4-fluorophenyl)boronic acid.

The title compound was isolated in 73% yield as a yellow solid. MS (ESI) m/z: 670.2 [M+1]+.

### Step 2: (S)-8-((3S,5R)-3,5-dimethylpiperazin-1-yl)-11-(4-fluorophenyl)-3-(pyridin-4-yl)-10-(trifluoromethyl)-3,4-dihydro-2H,6H-[1,4]thiazepino[2,3,4-ij]quinazolin-6-one

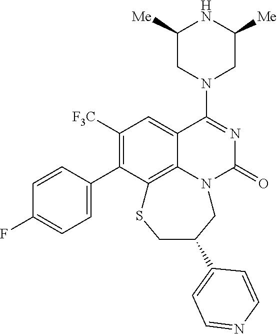 [SMILES: C[C@@H]1CN(c2nc(=O)n3c4c(c(-c5ccc(F)cc5)c(C(F)(F)F)cc24)SC[C@@H](c2ccncc2)C3)C[C@H](C)N1]

The title compound was prepared analogously to Example 100, step 10, where tert-butyl (2S,6R)-4-((3S)-11-(5-chloro-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((S)-11-(4-fluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate. The title compound was isolated in 99% yield as a yellow solid. MS (ESI) m/z: 570.1 [M+1]+

### Step 3: (S)-8-((3S,5R)-4-acryloyl-3,5-dimethylpiperazin-1-yl)-11-(4-fluorophenyl)-3-(pyridin-4-yl)-10-(trifluoromethyl)-3,4-dihydro-2H,6H-[1,4]thiazepino[2,3,4-ij]quinazolin-6-one

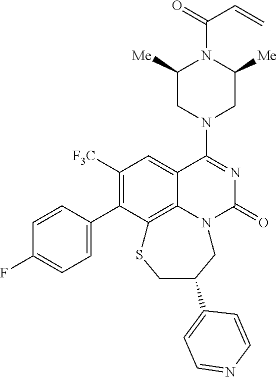

The title compound was prepared analogously to Example 100, step 11, where (3S)-11-(5-chloro-2,4-difluorophenyl)-8-((3S,5R)-3,5-dimethylpiperazin-1-yl)-3-(pyridin-4-yl)-10-(trifluoromethyl)-3,4-dihydro-2H,6H-[1,4]thiazepino[2,3,4-ij]quinazolin-6-one was replaced with (S)-8-((3S,5R)-3,5-dimethylpiperazin-1-yl)-11-(4-fluorophenyl)-3-(pyridin-4-yl)-10-(trifluoromethyl)-3,4-dihydro-2H,6H-[1,4]thiazepino[2,3,4-ij]quinazolin-6-one. The title compound was isolated in 91% yield as a yellow solid. MS (ESI) m/z: 624.2 [M+1]+. ¹H NMR (400 MHz, CDCl₃) δ ppm 1.53 (d, J=7.2 Hz, 3H) 1.61 (d, J=6.8 Hz, 3H) 3.01-3.18 (m, 1H) 3.41 (ddd, J=17.2, 13.2, 4.0 Hz, 2H) 3.60-3.83 (m, 2H) 4.20 (dd, J=12.8, 6.4 Hz, 2H) 4.56-4.95 (m, 4H) 5.74-5.85 (m, 1H) 6.43 (dd, J=16.8, 2.0 Hz, 1H) 6.59-6.71 (m, 1H) 7.17-7.26 (m, 4H) 7.29-7.38 (m, 2H) 8.13 (s, 1H) 8.49-8.71 (m, 2H).

### Example 114: (S)-8-((3S,5R)-4-acryloyl-3,5-dimethylpiperazin-1-yl)-11-(4-fluorophenyl)-3-(3-fluoropyridin-4-yl)-10-(trifluoromethyl)-3,4-dihydro-2H,6H-[1,4]thiazepino[2,3,4-ij]quinazolin-6-one

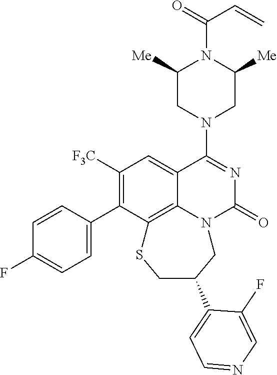

### Step 1: tert-butyl (2S,6R)-4-((S)-11-(4-fluorophenyl)-3-(3-fluoropyridin-4-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate

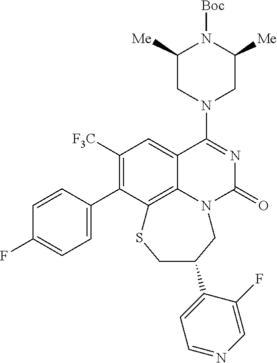

The title compound was prepared analogously to Example 100, step 9, where tert-butyl (2S,6R)-4-((S)-11-chloro-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate and (5-chloro-2,4-difluoro-phenyl)boronic acid were replaced with tert-butyl (2S,6R)-4-((S)-11-chloro-3-(3-fluoropyridin-4-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate and (4-fluorophenyl)boronic acid. The title compound was isolated as a yellow solid. MS (ESI) m/z: 688.2 [M+1]+.

### Step 2: (S)-8-((3S,5R)-3,5-dimethylpiperazin-1-yl)-11-(4-fluorophenyl)-3-(3-fluoropyridin-4-yl)-10-(trifluoromethyl)-3,4-dihydro-2H,6H-[1,4]thiazepino[2,3,4-ij]quinazolin-6-one

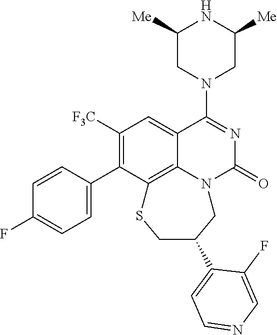 [SMILES: C[C@@H]1CN(c2nc(=O)n3c4c(c(-c5ccc(F)cc5)c(C(F)(F)F)cc24)SC[C@@H](c2ccncc2F)C3)C[C@H](C)N1]

The title compound was prepared analogously to Example 100, step 10, where tert-butyl (2S,6R)-4-((3S)-11-(5-chloro-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((S)-11-(4-fluorophenyl)-3-(3-fluoropyridin-4-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate. The title compound was isolated in 99% yield as a yellow solid. MS (ESI) m/z: 588.2 [M+1]+.

### Step 3: (S)-8-((3S,5R)-4-acryloyl-3,5-dimethylpiperazin-1-yl)-11-(4-fluorophenyl)-3-(3-fluoropyridin-4-yl)-10-(trifluoromethyl)-3,4-dihydro-2H,6H-[1,4]thiazepino[2,3,4-ij]quinazolin-6-one

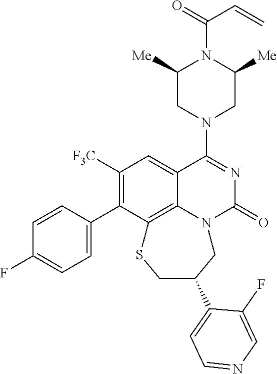

The title compound was prepared analogously to Example 100, step 11, where (3S)-11-(5-chloro-2,4-difluorophenyl)-8-((3S,5R)-3,5-dimethylpiperazin-1-yl)-3-(pyridin-4-yl)-10-(trifluoromethyl)-3,4-dihydro-2H,6H-[1,4]thiazepino[2,3,4-ij]quinazolin-6-one was replaced with (S)-8-((3S,5R)-3,5-dimethylpiperazin-1-yl)-11-(4-fluorophenyl)-3-(3-fluoropyridin-4-yl)-10-(trifluoromethyl)-3,4-dihydro-2H,6H-[1,4]thiazepino[2,3,4-ij]quinazolin-6-one. The title compound was isolated in 61% yield as a yellow solid. MS (ESI) m/z: 642.2 [M+1]+. ¹H NMR (400 MHz, CDCl³) δ ppm 1.53 (d, J=6.8 Hz, 3H) 1.60 (d, J=6.0 Hz, 3H) 2.91-3.20 (m, 1H) 3.34-3.48 (m, 2H) 3.53-3.81 (m, 1H) 3.97-4.26 (m, 3H) 4.45-5.07 (m, 4H) 5.73-5.85 (m, 1H) 6.43 (dd, J=16.8, 2.0 Hz, 1H) 6.58-6.69 (m, 1H) 7.14-7.23 (m, 3H) 7.25 (br s, 1H) 7.30 (br s, 1H) 8.12 (s, 1H) 8.29-8.57 (m, 2H).

### Example 115: 8-((3S,5R)-4-acryloyl-3,5-dimethylpiperazin-1-yl)-3,11-bis(4-fluorophenyl)-10-(trifluoromethyl)-3,4-dihydro-2H,6H-[1,4]thiazepino[2,3,4-iij]quinazolin-6-one

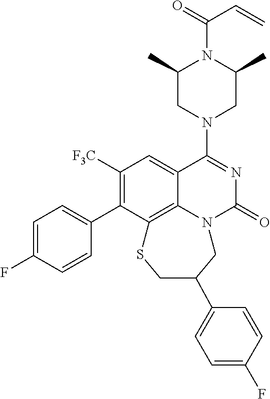

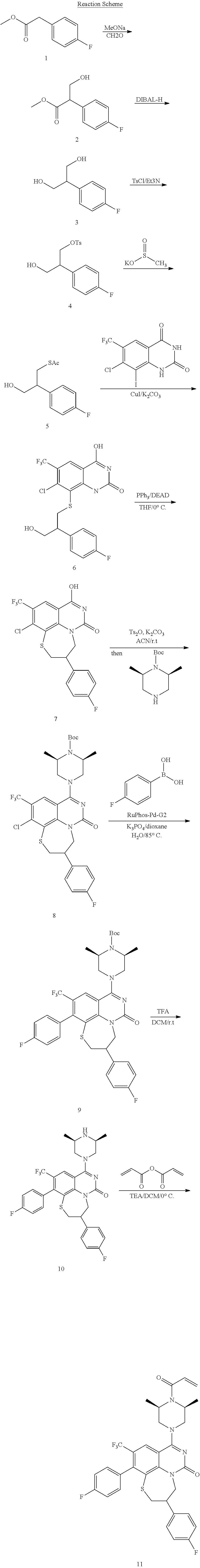

### Methyl 2-(4-fluorophenyl)-3-hydroxypropanoate (2)

To a mixture of methyl 2-(4-fluorophenyl)acetate (5 g, 29.76 mmol) and sodium methanolate (0.08031 g, 1.49 mmol) in DMSO (10 mL) was added polyformaldehyde (0.93 g, 31.25 mmol) at 20° C. for 16 hours. The mixture was quenched with water and neutralized with 2 N hydrochloric acid, and extracted with ethyl acetate (100 mL×3). The combined organic phases was washed with brine (50 mL) and dried over anhydrous sodium sulfate. After filtration and concentration, the residue was purified by silica gel column with petroleum ether/ethyl acetate=5%˜40% to afford methyl 2-(4-fluorophenyl)-3-hydroxypropanoate 2 (4.5 g, yield 76%) as a white solid.

### 2-(4-fluorophenyl)propane-1,3-diol (3)

To a solution of methyl 2-(4-fluorophenyl)-3-hydroxypropanoate (11 g, 55.56 mmol) in THF (100 mL) was added 1.0 M DIBAL-H (166.68 mmol, 166.68 mL) at −78° C. under N2 atmosphere. The mixture was stirred at −78° C. under nitrogen atmosphere for 3 hours. After completion, the mixture was quenched with sodium sulfate decahydrate and filtration. The solution was concentrated and the residue was purified by silica gel column (PE/EA=10%˜100%) to afford 2-(4-fluorophenyl)propane-1,3-diol 3 (5.05 g, yield 53%) as a yellow oil.

### 2-(4-fluorophenyl)-3-hydroxypropyl 4-methylbenzenesulfonate (4)

To a mixture of 2-(4-fluorophenyl)propane-1,3-diol (5.0 g, 29.4 mmol) and TEA (5.94 g, 58.82 mmol) in DCM (50 mL) was added Tosyl chloride (3.35 g, 17.6 mmol) at 0° C. The mixture was stirred at 20° C. for 12 hours. After completion, the mixture was concentrated under reduced pressure and the residue was purified by silica gel column (PE/EA=0˜2:1) to afford 2-(4-fluorophenyl)-3-hydroxypropyl 4-methylbenzenesulfonate 4 (3.58 g, yield 38%) as a white solid.

### S-(2-(4-fluorophenyl)-3-hydroxypropyl) ethanethioate (5)

To a mixture of 2-(4-fluorophenyl)-3-hydroxypropyl 4-methylbenzenesulfonate (3.6 g, 11.11 mmol) in acetone (10 mL) was added potassium methanesulfinate (2.53 g, 22.2 mmol) at 20° C. The mixture was stirred at 50° C. for 1 hour. After completion, the mixture was concentrated under reduced pressure and the residue was purified by silica gel column (DCM/MeOH=0˜20:1) to afford S-(2-(4-fluorophenyl)-3-hydroxypropyl) ethanethioate 5 (1.7 g, yield 67%) as a yellow oil.

### 7-chloro-8-((2-(4-fluorophenyl)-3-hydroxypropyl)thio)-4-hydroxy-6-(trifluoromethyl)quinazolin-2(1H)-one (6)

To a mixture of S-(2-(4-fluorophenyl)-3-hydroxypropyl) ethanethioate (1.65 g, 7.24 mmol) and 7-chloro-8-iodo-6-(trifluoromethyl)quinazoline-2,4(1H,3H)-dione (2.82 g, 7.24 mmol) in ethylene glyol (10 mL) and isopropanol (10 mL) were added CuI (551 mg, 2.90 mmol) and K2CO3 (3 g, 21.72 mmol) at 20° C. under N2 atmosphere. The mixture was stirred at 85° C. for 3 hours. After completion, the mixture was extracted with ethyl acetate (100 mL×3). The combined organic phases was washed with brine (50 mL) and dried over anhydrous sodium sulfate. The organic was concentrated under reduced pressure and the residue was purified by silica gel column with DCM/MeOH=0%˜20% to afford 7-chloro-8-((2-(4-fluorophenyl)-3-hydroxypropyl)thio)-4-hydroxy-6-(trifluoromethyl)quinazolin-2(1H)-one 6 (160 mg) as a yellow solid.

### 11-chloro-3-(4-fluorophenyl)-8-hydroxy-10-(trifluoromethyl)-3,4-dihydro-2H,6H-[1,4]thiazepino[2,3,4-ij]quinazolin-6-one (7)

To a mixture of 7-chloro-8-((2-(4-fluorophenyl)-3-hydroxypropyl)thio)-4-hydroxy-6-(trifluoromethyl)quinazolin-2(1H)-one (160 mg, 0.36 mmol) and triphenylphosphine (189 mg, 0.72 mmol) in tetrahydrofuran (20 mL) was added diethyl azodicarboxylate (125 mg, 0.72 mmol) at 0° C. under N2 atmosphere. The mixture was stirred at 20° C. for 1 hour. After completion, the mixture was concentrated under reduced pressure and the residue was purified by the reversed flash to afford 11-chloro-3-(4-fluorophenyl)-8-hydroxy-10-(trifluoromethyl)-3,4-dihydro-[1,4]thiazepino[2,3,4-ij]quinazolin-6(2H)-one 7 (100 mg, 73% yield) as a yellow solid.

### tert-butyl (2S,6R)-4-(11-chloro-3-(4-fluorophenyl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate (8)

To a mixture of 11-chloro-3-(4-fluorophenyl)-8-hydroxy-10-(trifluoromethyl)-3,4-dihydro-[1,4]thiazepino[2,3,4-ij]quinazolin-6(2H)-one 7 (100 mg, 0.23 mmol) and potassium carbonate (254 mg, 1.84 mmol) in acetonitrile (10 mL) was added 4-methylbenzenesulfonic anhydride (112 mg, 0.345 mmol) at 0° C. The mixture was stirred at 30° C. for 4 hours. Then, (2S,6R)-tert-butyl 2,6-dimethylpiperazine-1-carboxylate (98 mg, 0.46 mmol) was added into the reaction mixture. The reaction mixture was stirred at 30° C. for further 2 hours. After completion, the mixture was concentrated under reduced pressure, and the residue was purified by silica gel column (PE/EA=0%˜100%) to afford tert-butyl (2S,6R)-4-(11-chloro-3-(4-fluorophenyl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate 8 (50 mg, 35% yield)) as a yellow solid.

### tert-butyl (2S,6R)-4-(3,11-bis(4-fluorophenyl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate (9)

To a solution of tert-butyl (2S,6R)-4-(11-chloro-3-(4-fluorophenyl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate 8 (50 mg, 0.08 mmol), (4-fluorophenyl)boronic acid (90 mg, 0.64 mmol) and tripotassium phosphate (64 mg, 0.24 mmol) in 1,4-dioxane (4 mL) and water (1 mL) was added chloro(2-dicyclohexylphosphino-2′, 6′-diisopropoxy-1,1′-biphenyl)[2-(2′-amino-1,1′-biphenyl)]palladium(II) (12 mg, 0.016 mmol) at 20° C. under N2 atmosphere. The mixture was stirred at 80° C. under nitrogen atmosphere for 2 h. After completion, the mixture was concentrated under reduced pressure and the residue was purified by silica gel column (DCM/MeOH=0%˜20%) to afford tert-butyl (2S,6R)-4-(3,11-bis(4-fluorophenyl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate 9 (40 mg, yield: 73%) as a yellow solid.

### 8-((3S,5R)-3,5-dimethylpiperazin-1-yl)-3,11-bis(4-fluorophenyl)-10-(trifluoromethyl)-3,4-dihydro-2H,6H-[1,4]thiazepino[2,3,4-ij]quinazolin-6-one (10)

To a solution of tert-butyl (2S,6R)-4-(3,11-bis(4-fluorophenyl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate 9 (40 mg, 0.058 mmol) in dichloromethane (5 ml) was added trifluoroacetic acid (5 mL) at 0° C. The reaction solution was stirred at room temperature for 1 h. After completion, the mixture was concentrated and the residue was purified by silica gel column (dichloromethane/methanol=0%˜20%) to afford the product 10 (20 mg, yield: 59%) as a yellow solid.

### 8-((3S,5R)-4-acryloyl-3,5-dimethylpiperazin-1-yl)-3,11-bis(4-fluorophenyl)-10-(trifluoromethyl)-3,4-dihydro-2H,6H-[1,4]thiazepino[2,3,4-ij]quinazolin-6-one (11)

To a mixture of 8-((3S,5R)-3,5-dimethylpiperazin-1-yl)-3,11-bis(4-fluorophenyl)-10-(trifluoromethyl)-3,4-dihydro-[1,4]thiazepino[2,3,4-ij]quinazolin-6(2H)-one 10 (20 mg, 0.034 mmol) and triethyl amine (10 mg, 0.102 mmol) in dichloromethane (5 ml) was added acrylic anhydride (9 mg, 0.068 mmol) at 0° C. The mixture was stirred at 0° C. for 1 hour. After completion, the mixture was concentrated and the residue was purified by Prep-HPLC to afford 8-((3S,5R)-4-acryloyl-3,5-dimethylpiperazin-1-yl)-3,11-bis(4-fluorophenyl)-10-(trifluoromethyl)-3,4-dihydro-[1,4]thiazepino[2,3,4-ij]quinazolin-6(2H)-one 11 (7 mg, yield: 32%) as a white solid. ¹H NMR (400 MHz, CDCl₃) δ 8.11 (s, 1H), 7.35-7.29 (m, 3H), 7.25-7.16 (m, 3H), 7.04-6.99 (m, 2H), 6.67-6.60 (m, 1H), 6.42 (dd, J=16.8 Hz, 2.0 Hz, 1H), 5.78 (dd, J=10.8 Hz, 2.4 Hz, 1H), 4.78-4.63 (m, 4H), 4.21-4.15 (m, 2H), 3.75-3.63 (m, 2H), 3.42-3.33 (m, 2H), 3.06-3.03 (m, 1H), 1.60 (d, J=6.8 Hz, 3H), 1.51 (d, J=6.8 Hz, 3H).

### Example 122: (R)-8-((3S,5R)-4-acryloyl-3,5-dimethylpiperazin-1-yl)-11-(4-fluorophenyl)-3-(thiophen-3-yl)-10-(trifluoromethyl)-3,4-dihydro-[1,4]thiazepino[2,3,4-ij]quinazolin-6(2H)-one (P1) and Example 123: (S)-8-((3S,5R)-4-acryloyl-3,5-dimethylpiperazin-1-yl)-11-(4-fluorophenyl)-3-(thiophen-3-yl)-10-(trifluoromethyl)-3,4-dihydro-[1,4]thiazepino[2,3,4-ij]quinazolin-6(2H)-one (P2)

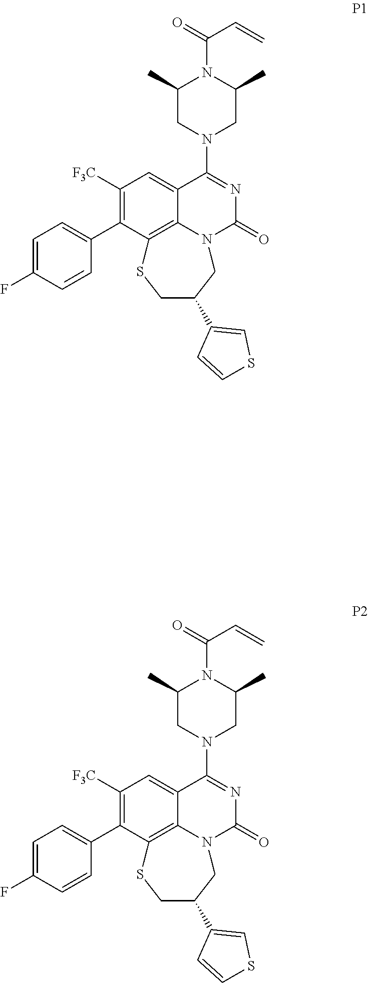

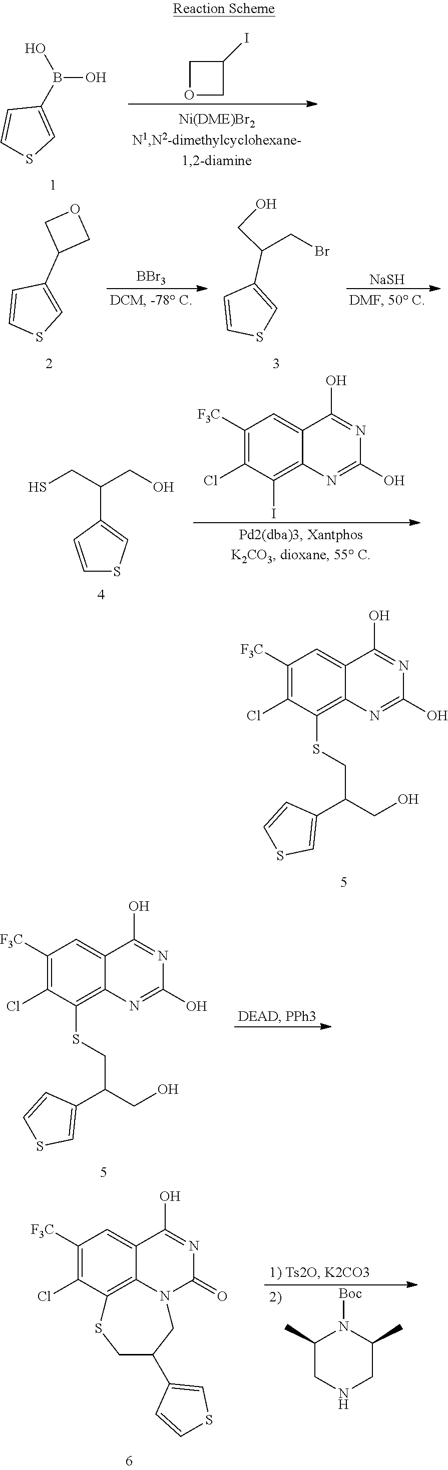

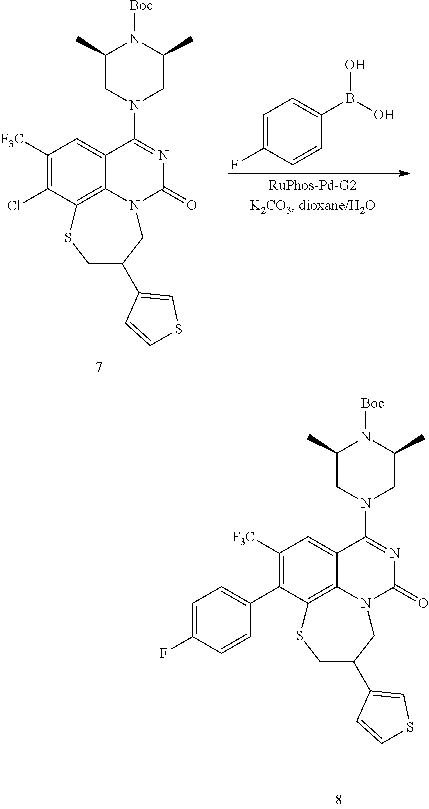 [SMILES: C[C@@H]1CN(c2nc(=O)n3c4c(c(Cl)c(C(F)(F)F)cc24)SCC(c2ccsc2)C3)C[C@H](C)N1C(=O)OC(C)(C)C ; OB(O)c1ccc(F)cc1 ; C[C@@H]1CN(c2nc(=O)n3c4c(c(-c5ccc(F)cc5)c(C(F)(F)F)cc24)SCC(c2ccsc2)C3)C[C@H](C)N1C(=O)OC(C)(C)C]

### 3-(thiophen-3-yl)oxetane (2)

To a mixture of thiophen-3-ylboronic acid (20 g, 101 mmol), Nickel(II) bromide ethylene glycol dimethyl ether complex (3.12 g, 10.1 mol), (1S,2S)—N¹,N²-dimethylcyclohexane-1,2-diamine (2.15 g, 15.15 mmol) and Potassium tert-butoxide (22.62 g, 202 mmol) in 1,4-dioxane (350 mL) was added 3-iodooxetane (27.88 g, 151.5 mmol) at 0° C. under nitrogen atmosphere. The mixture was stirred at 60° C. for 18 hours. After completion, the mixture was poured into ice-water (300 mL) and extracted with ethyl acetate (150 mL×3). After concentration, the residue was purified by silica gel column with petroleum ether/ethyl acetate=20/1 to afford to afford 3-(thiophen-3-yl)oxetane (2) (1.38 g, 10% yield) as a colorless oil. ¹H NMR (400 MHz, CDCl₃) δ 7.35-7.33 (m, 1H), 7.20-7.18 (m, 1H), 7.12-7.11 (m, 1H), 5.04-5.00 (m, 2H), 4.77-4.74 (m, 2H), 4.35-4.27 (m, 1H).

### 3-bromo-2-(thiophen-3-yl)propan-1-ol (3)

To a solution of 3-(thiophen-3-yl)oxetane (2) (1.38 g, 9.86 mmol) in dichloromethane (30 mL) was added Boron tribromide (10 mL, 9.86 mmol, 1M in dichloromethane) at −78° C. under nitrogen atmosphere. The mixture was stirred at 0° C. for 1 hour. After completion, the mixture was poured into ice-water (50 mL) and extracted with ethyl acetate (50 mL×3), the mixture was concentrated to afford the product 3-bromo-2-(thiophen-3-yl)propan-1-ol (3) (2.1 g, crude) as colorless oil. ¹H NMR (400 MHz, CDCl₃) δ 7.26-7.24 (m, 1H), 7.07 (d, J=2.0 Hz, 1H), 6.94 (d, J=4.8 Hz, 1H), 3.88-3.81 (m, 2H), 3.68-3.55 (m, 2H), 3.27-3.21 (m, 1H).

### 3-mercapto-2-(thiophen-3-yl)propan-1-ol (4)

To a solution of 3-bromo-2-(thiophen-3-yl)propan-1-ol (3) (2.1 g 9.54 mmol) in N,N-dimethylformamide (30 mL) was added sodium hydrogensulfide (1.6 g, 28.62 mmol). The mixture was stirred at room temperature for 2 hours. After completion, the mixture was poured into ice-water (50 mL) and extracted with ethyl acetate (50 mL×3). After concentration, the residue was purified by silica gel column with dichloromethane/methanol=30/1 to afford to afford 3-mercapto-2-(thiophen-3-yl)propan-1-ol (4) (1.42 g, 85% yield). ¹H NMR (400 MHz, CDCl₃) δ 7.35-7.33 (m, 1H), 7.11 (d, J=2.0 Hz, 1H), 7.00 (d, J=5.2 Hz, 1H), 3.87 (d, J=6.0 Hz, 2H), 3.16-3.12 (m, 1H), 2.91-2.83 (m, 2H).

### 7-chloro-8-((3-hydroxy-2-(thiophen-3-yl)propyl)thio)-6-(trifluoromethyl)quinazoline-2,4-diol (5)

To a solution of 7-chloro-8-iodo-6-(trifluoromethyl)quinazoline-2,4-diol (2.12 g, 5.44 mmol) in 1,4-dioxane (70 mL), potassium carbonate (1.5 g, 10.88 mmol), 3-mercapto-2-(thiophen-3-yl)propan-1-ol (4) (1.42 g, 8.16 mmol), 4,5-Bis(diphenyl-phosphino)-9,9-dimethylxanthene (472 mg, 0.816 mmol) and Tris(dibenzylideneacetone) dipalladium (498 mg, 0.544 mmol) were added. The mixture was stirred at 60° C. under nitrogen atmosphere for 18 hours. After completion, the mixture was concentrated under reduced pressure, the residue was purified by silica gel column chromatography (dichloromethane/ethyl acetate=4/1) to afford 7-chloro-8-((3-hydroxy-2-(thiophen-3-yl)propyl)thio)-6-(trifluoromethyl)quinazoline-2,4-diol (5) (1.0 g, 42% yield) as pale yellow solid. MS (ESI) m/z 435.1 [M−H]⁺.

### 11-chloro-8-hydroxy-3-(thiophen-3-yl)-10-(trifluoromethyl)-3,4-dihydro-[1,4]thiazepino[2,3,4-ij]quinazolin-6(2H)-one (6)

To a mixture of 7-chloro-8-((3-hydroxy-2-(thiophen-3-yl)propyl)thio)-6-(trifluoromethyl)quinazoline-2,4-diol (5) (1.0 g, 2.29 mmol) and triphenylphosphine (1.2 g, 4.58 mmol) in tetrahydrofuran (40 mL) was added diethyl azodicarboxylate (797 mg, 4.58 mmol) at 0° C. The mixture was stirred at 0° C. for 45 min. After completion, the mixture was poured into ice-water (50 mL) and extracted with ethyl acetate (50 mL×3). Concentrated and the residue was purified by C18 with 30-95% acetonitrile in water to afford 11-chloro-8-hydroxy-3-(thiophen-3-yl)-10-(trifluoromethyl)-3,4-dihydro-[1,4]thiazepino[2,3,4-ij]quinazolin-6(2H)-one (6) (610 mg, 64% yield) as a yellow solid. MS (ESI) m/z 417.1 [M−H]⁺.

### (2S,6R)-tert-butyl 4-(11-chloro-6-oxo-3-(thiophen-3-yl)-10-(trifluoromethyl)-2,3,4,6-tetrahydro-[1,4]thiazepino[2,3,4-ij]quinazolin-8-yl)-2,6-dimethylpiperazine-1-carboxylate (7)

To a mixture of 11-chloro-8-hydroxy-3-(thiophen-3-yl)-10-(trifluoromethyl)-3,4-dihydro-[1,4]thiazepino[2,3,4-ij]quinazolin-6(2H)-one (6) (610 mg, 1.46 mmol) and potassium carbonate (2.01 g, 14.6 mmol) in acetonitrile (50 mL) was added 2,4,6-Tris(prop-2-yl)benzenesulphonyl chloride (675 mg, 2.19 mmol). The mixture was stirred at 35° C. for 4 hours. After completion, (2S,6R)-tert-butyl 2,6-dimethylpiperazine-1-carboxylate (625 mg, 2.92 mmol) was added into the reaction solution. The reaction mixture was stirred at 35° C. for 1 hour. After completion, the mixture was poured into ice-water (50 mL) and extracted with ethyl acetate (50 mL×3). Concentrated and the residue was purified by C18 column with 20-95% acetonitrile in water to afford (2S,6R)-tert-butyl 4-(11-chloro-6-oxo-3-(thiophen-3-yl)-10-(trifluoromethyl)-2,3,4,6-tetrahydro-[1,4]thiazepino[2,3,4-ij]quinazolin-8-yl)-2,6-dimethylpiperazine-1-carboxylate (7) (630 mg, 70% yield) as a pale yellow solid. MS (ESI) m/z 615.2[M+H]⁺.

### (2S,6R)-tert-butyl 4-(11-(4-fluorophenyl)-6-oxo-3-(thiophen-3-yl)-10-(trifluoromethyl)-2,3,4,6-tetrahydro-[1,4]thiazepino[2,3,4-ij]quinazolin-8-yl)-2,6-dimethylpiperazine-1-carboxylate (8)

To a solution of (2S,6R)-tert-butyl 4-(11-chloro-6-oxo-3-(thiophen-3-yl)-10-(trifluoromethyl)-2,3,4,6-tetrahydro-[1,4]thiazepino[2,3,4-ij]quinazolin-8-yl)-2,6-dimethylpiperazine-1-carboxylate (7) (180 mg, 0.29 mmol) in 1,4-dioxane (3 mL) and water (0.5 mL), tripotassium phosphate (120 mg, 0.87 mmol), (4-fluorophenyl)boronic acid (162 mg, 1.16 mmol), and chloro(2-dicyclohexylphosphino-2′,6′-diisopropoxy-1,1′-biphenyl)[2-(2′-amino-1,1′-biphenyl)]palladium(II) (23 mg, 0.03 mmol) were added. The mixture was stirred at 80° C. under nitrogen atmosphere for 2 hours. After completion, the mixture was concentrated under reduced pressure, the residue was purified by silica gel column chromatography (dichloromethane/methanol=50/1) to afford (2S,6R)-tert-butyl 4-(11-(4-fluorophenyl)-6-oxo-3-(thiophen-3-yl)-10-(trifluoromethyl)-2,3,4,6-tetrahydro-[1,4]thiazepino[2,3,4-ij]quinazolin-8-yl)-2,6-dimethylpiperazine-1-carboxylate (8) (210 mg, crude) as a yellow solid. MS (ESI) m/z 675.1 [M+H]⁺.

### 8-((3S,5R)-3,5-dimethylpiperazin-1-yl)-11-(4-fluorophenyl)-3-(thiophen-3-yl)-10-(trifluoromethyl)-3,4-dihydro-[1,4]thiazepino[2,3,4-ij]quinazolin-6(2H)-one (9)

To a mixture of (2S,6R)-tert-butyl 4-(11-(4-fluorophenyl)-6-oxo-3-(thiophen-3-yl)-10-(trifluoromethyl)-2,3,4,6-tetrahydro-[1,4]thiazepino[2,3,4-ij]quinazolin-8-yl)-2,6-dimethylpiperazine-1-carboxylate (8) (210 mg, crude) in dichloromethane (2 mL) was added trifluoroacetic acid (2 mL) at 0° C. The reaction solution was stirred at room temperature for 1 hour. After completion, the mixture was concentrated and the residue was purified by silica gel column chromatography (dichloromethane/methanol=15/1) to afford 8-((3S,5R)-3,5-dimethylpiperazin-1-yl)-11-(4-fluorophenyl)-3-(thiophen-3-yl)-10-(trifluoromethyl)-3,4-dihydro-[1,4]thiazepino[2,3,4-ij]quinazolin-6(2H)-one (9) (160 mg, crude) as a pale yellow solid. MS (ESI) m/z 575.1[M+H]⁺.

### 8-((3S,5R)-4-acryloyl-3,5-dimethylpiperazin-1-yl)-11-(4-fluorophenyl)-3-(thiophen-3-yl)-10-(trifluoromethyl)-3,4-dihydro-[1,4]thiazepino[2,3,4-ij]quinazolin-6(2H)-one (10)

To a mixture of 8-((3S,5R)-3,5-dimethylpiperazin-1-yl)-11-(4-fluorophenyl)-3-(thiophen-3-yl)-10-(trifluoromethyl)-3,4-dihydro-[1,4]thiazepino[2,3,4-ij]quinazolin-6(2H)-one (9) (160 mg, 0.28 mmol) and triethyl amine (57 mg, 0.56 mmol) in dichloromethane (4 ml) was added acrylic anhydride (53 mg, 0.42 mmol) at 0° C. The mixture was stirred at 0° C. for 1 hour. After completion, the mixture was poured into ice-water (10 mL) and extracted with dichloromethane (10 mL×3). Concentrated and the residue was purified by preparative High Performance Liquid Chromatography (20% to 95% acetonitrile in water) to afford 8-((3S,5R)-4-acryloyl-3,5-dimethylpiperazin-1-yl)-11-(4-fluorophenyl)-3-(thiophen-3-yl)-10-(trifluoromethyl)-3,4-dihydro-[1,4]thiazepino[2,3,4-ij]quinazolin-6(2H)-one (10) (70 mg, 40% yield) as a white solid. MS (ESI) m/z 629.2 [M+H]⁺.

The above racemate (70 mg) was dissolved in ethanol (5 mL) and separated by chiral supercritical fluid chromatography (separation condition: Column: IF 5 μm 20×250 mm; Mobile Phase: MeOH/DCM=90/10 at 25 mL/min; Temp: 30° C.; Wavelength: 254 nm) to afford the title compounds P1 (20 mg, yield: 29%, 100% ee) and P2 (22 mg, yield: 31%, 99.6% ee); Chiral HPLC Analytical: on IF was using 5 μm 4.6×250 mm column, Mobile Phase: MeOH/DCM=90/10 at 1 mL/min; temperature: 30° C.; Wavelength: 254 nm).

### (R)-8-((3S,5R)-4-acryloyl-3,5-dimethylpiperazin-1-yl)-11-(4-fluorophenyl)-3-(thiophen-3-yl)-10-(trifluoromethyl)-3,4-dihydro-[1,4]thiazepino[2,3,4-ij]quinazolin-6(2H)-one (P1)

¹H NMR (400 MHz, CDCl₃) δ 8.10 (s, 1H), 7.31-7.29 (m, 1H), 7.24 (s, 1H), 7.20-7.15 (m, 4H), 7.10 (d, J=4.8 Hz, 1H), 6.63 (dd, J=16.4 Hz, 10.8 Hz, 1H), 6.42 (dd, J=16.8 Hz, 1.6 Hz, 1H), 5.78 (dd, J=10.4 Hz, 1.6 Hz, 1H), 4.87-4.60 (m, 4H), 4.17 (t, J=12.0 Hz, 2H), 3.89-3.85 (m, 1H), 3.62 (d, J=10.4 Hz, 1H), 3.41-3.32 (m, 2H), 3.07 (d, J=14.8 Hz, 1H), 1.60 (d, J=7.2 Hz, 3H), 1.51 (d, J=6.8 Hz, 3H); Chiral HPLC fraction 1: e.e. =100%, Rt=7.610 min.

### (S)-8-((3S,5R)-4-acryloyl-3,5-dimethylpiperazin-1-yl)-11-(4-fluorophenyl)-3-(thiophen-3-yl)-10-(trifluoromethyl)-3,4-dihydro-[1,4]thiazepino[2,3,4-ij]quinazolin-6(2H)-one (P2)

¹H NMR (400 MHz, CDCl₃) δ 8.10 (s, 1H), 7.31-7.29 (m, 1H), 7.24 (s, 1H), 7.20-7.15 (m, 4H), 7.10 (d, J=4.0 Hz, 1H), 6.63 (dd, J=16.0 Hz, 10.0 Hz, 1H), 6.42 (dd, J=16.4 Hz, 1.6 Hz, 1H), 5.78 (dd, J=10.4 Hz, 1.6 Hz, 1H), 4.85-4.60 (m, 4H), 4.18 (t, J=11.6 Hz, 2H), 3.88-3.84 (m, 1H), 3.62 (d, J=8.8 Hz, 1H), 3.41-3.32 (m, 2H), 3.07 (d, J=16.4 Hz, 1H), 1.60 (d, J=7.2 Hz, 3H), 1.51 (d, J=6.8 Hz, 3H); Chiral HPLC fraction 1: e.e. =99.6%, Rt=9.489 min.

### Example 124: (R)-8-((3S,5R)-4-acryloyl-3,5-dimethylpiperazin-1-yl)-11-(4-fluorophenyl)-3-(thiophen-2-yl)-10-(trifluoromethyl)-3,4-dihydro-2H,6H-[1,4]thiazepino[2,3,4-ij]quinazolin-6-one (P1)

### Example 125: (S)-8-((3S,5R)-4-acryloyl-3,5-dimethylpiperazin-1-yl)-11-(4-fluorophenyl)-3-(thiophen-2-yl)-10-(trifluoromethyl)-3,4-dihydro-2H,6H-[1,4]thiazepino[2,3,4-ij]quinazolin-6-one (P2)

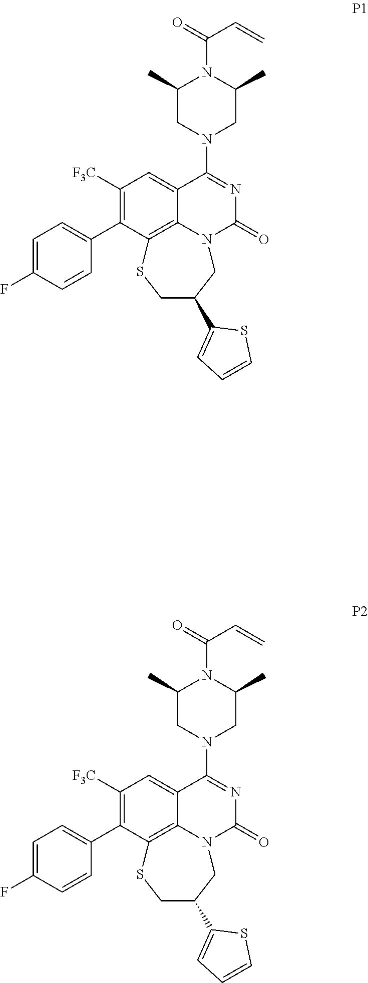

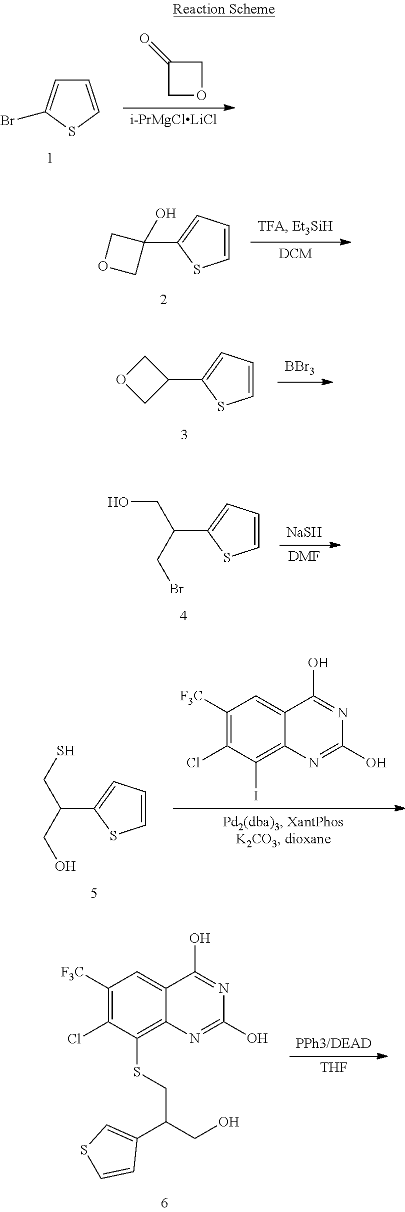

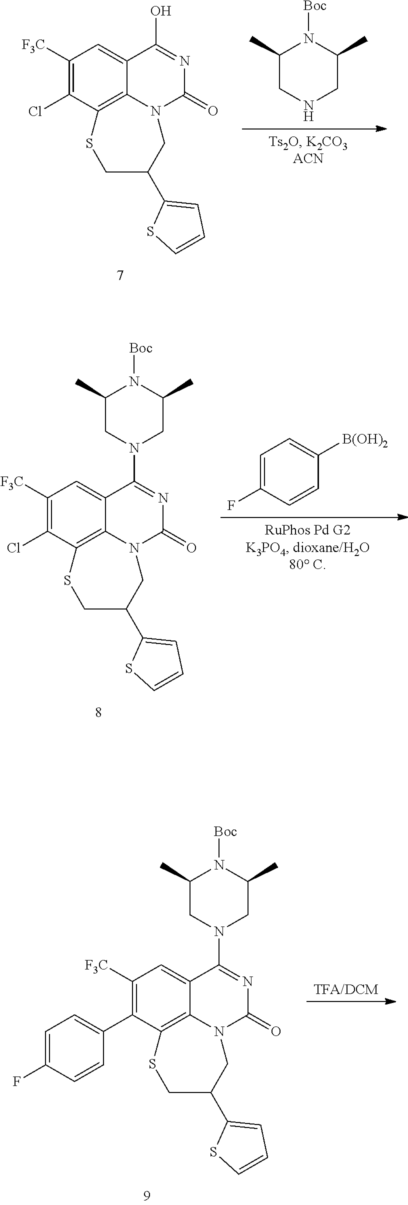

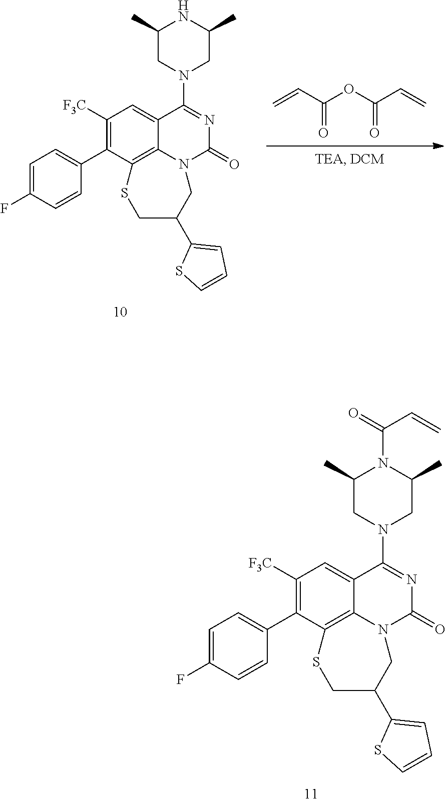

### 3-(thiophen-2-yl)oxetan-3-ol (2)

To a solution of 2-bromothiophene (10 g, 61.35 mmol) in tetrahydrofuran (205 mL) was added isopropyl magnesium chloride lithium chloride solution (49.6 mL, 64.42 mmol, 1.3M tetrahydrofuran) at −78° C. under nitrogen atmosphere. The mixture was stirred at −78° C. for 1 hour. Then oxetan-3-one (5.3 g, 73.62 mmol) was added slowly. The mixture was slowly warmed up to room temperature and stirred for 2 hours. Saturated ammonium chloride aqueous solution (200 mL) was added slowly and extracted with ethyl acetate (300 mL×3). After concentration, the residue was purified by silica gel column (with petroleum ether/ethyl acetate=3/1) to afford 3-(thiophen-2-yl)oxetan-3-ol (2) (6.9 g, 72% yield) as a pale yellow oil. ¹H NMR (400 MHz, CDCl₃) δ 7.31-7.30 (m, 1H), 7.19-7.18 (m, 1H), 7.05-7.04 (m, 1H), 4.93-2.89 (m, 4H).

### 3-(thiophen-2-yl)oxetane (3)

To a mixture of 3-(thiophen-2-yl)oxetan-3-ol (2) (6.9 g, 44.17 mmol) and trifluoroacetic acid (15.4 mL, 200.85 mmol) in dichloromethane (75 mL) was added triethylsilane (32.1 mL, 200.85 mmol) at 0° C. The mixture was stirred at 30° C. for 3 hours. After completion, the mixture was poured into ice-water (150 mL) and extracted with dichloromethane (150 mL×3). After concentration, the residue was purified by silica gel column (with petroleum ether/ethyl acetate=10/1) to afford 3-(thiophen-2-yl)oxetane (3) (5.8 g, 94% yield) as a colorless oil. ¹H NMR (400 MHz, CDCl₃) δ 7.23-7.21 (m, 1H), 7.00-6.96 (m, 2H), 5.06-5.02 (m, 2H), 4.82-2.79 (m, 2H), 4.56-4.48 (m, 1H).

### 3-bromo-2-(thiophen-2-yl)propan-1-ol (4)

To a solution of 3-(thiophen-2-yl)oxetane (3) (5.8 g, 41.37 mmol) in dichloromethane (140 mL) was added slowly boron tribromide (41.4 mL, 41.4 mmol, 1.0 M in dichloromethane) at −78° C. under nitrogen atmosphere. The mixture was stirred at −78° C. for 2 hours. After completion, the mixture was poured into ice-water (150 mL) and extracted with dichloromethane (150 mL×3). After concentration, the residue was purified by silica gel column (with petroleum ether/ethyl acetate=2/1) to afford 3-bromo-2-(thiophen-2-yl)propan-1-ol (4) (5.3 g, 58% yield). ¹H NMR (400 MHz, CDCl₃) δ 7.25-7.23 (m, 1H), 7.01-6.99 (m, 1H), 6.97-6.96 (m, 1H), 3.97-3.96 (m, 2H), 3.74-3.64 (m, 2H), 3.51-3.48 (m, 1H).

### 3-mercapto-2-(thiophen-2-yl)propan-1-ol (5)

To a solution of 3-bromo-2-(thiophen-2-yl)propan-1-ol (4) (5.3 g, 23.97 mmol) in N,N-dimethylformamide (48 mL), sodium bisulfide (5.76 g, 71.91 mmol, 70%) was added at 0° C. The mixture was stirred at 30° C. under nitrogen atmosphere for 4 hours.

After completion, the mixture was poured into ice-water (200 mL) and extracted with ethyl acetate (200 mL×3). After concentration, the residue was purified by silica gel column (with petroleum ether/ethyl acetate=2/1) to afford 3-mercapto-2-(thiophen-2-yl)propan-1-ol (5) (2.5 g, 60% yield) as pale yellow oil. ¹H NMR (400 MHz, CDCl₃) δ 7.23-7.22 (m, 1H), 7.00-6.98 (m, 1H), 6.93-6.92 (m, 1H), 3.89-3.87 (m, 2H), 3.32-3.26 (m, 1H), 2.95-2.82 (m, 2H), 1.44-1.40 (m, 1H).

### 7-chloro-8-((3-hydroxy-2-(thiophen-3-yl)propyl)thio)-6-(trifluoromethyl)quinazoline-2,4-diol (6)

To a solution of 7-chloro-8-iodo-6-(trifluoromethyl)quinazoline-2,4-diol (2.24 g, 5.74 mmol) in 1,4-dioxane (60 mL), potassium carbonate (2.38 g, 17.22 mmol), 3-mercapto-2-(thiophen-2-yl)propan-1-ol (5) (2.5 g, 14.34 mmol), 4,5-Bis(diphenyl-phosphino)-9,9-dimethylxanthene (498 mg, 0.86 mmol) and Tris(dibenzylideneacetone) dipalladium (522 mg, 0.57 mmol) were added. The mixture was stirred at 60° C. under nitrogen atmosphere for 18 hours. After completion, the mixture was concentrated under reduced pressure, the residue was purified by silica gel column chromatography (dichloromethane/methanol=30/1) to afford 7-chloro-8-((3-hydroxy-2-(thiophen-3-yl)propyl)thio)-6-(trifluoromethyl)quinazoline-2,4-diol (6) (2.2 g, crude) as pale yellow solid. MS (ESI) m/z 435.0 [M−H]⁻.

### 11-chloro-8-hydroxy-3-(thiophen-2-yl)-10-(trifluoromethyl)-3,4-dihydro-2H,6H-[1,4]thiazepino[2,3,4-ij]quinazolin-6-one (7)

To a mixture of 7-chloro-8-((3-hydroxy-2-(thiophen-3-yl)propyl)thio)-6-(trifluoromethyl)quinazoline-2,4-diol (6) (2.2 g, 5.04 mmol) and triphenylphosphine (2.64 g, 10.08 mmol) in tetrahydrofuran (100 mL) was added diethyl azodicarboxylate (1.76 g, 10.08 mmol) at 0° C. under nitrogen atmosphere. The mixture was stirred at room temperature for 3 hours. After completion, the mixture was poured into ice-water (100 mL) and extracted with ethyl acetate (100 mL×3). The organic phase was concentrated and the residue was purified by C18 with 30-95% acetonitrile in water to afford 11-chloro-8-hydroxy-3-(thiophen-2-yl)-10-(trifluoromethyl)-3,4-dihydro-2H,6H-[1,4]thiazepino[2,3,4-ij]quinazolin-6-one (7) (665 mg, 28% yield for two steps) as a yellow solid. MS (ESI): m/z 419.2 [M+H]⁺.

### tert-butyl (2S,6R)-4-(11-chloro-6-oxo-3-(thiophen-2-yl)-10-(trifluoromethyl)-3,4-dihydro-2H,6H-[1,4]thiazepino[2,3,4-ij]quinazolin-8-yl)-2,6-dimethylpiperazine-1-carboxylate (8)

To a mixture of 11-chloro-8-hydroxy-3-(thiophen-2-yl)-10-(trifluoromethyl)-3,4-dihydro-2H,6H-[1,4]thiazepino[2,3,4-ij]quinazolin-6-one (7) (665 mg, 1.59 mmol) and potassium carbonate (1.10 g, 7.95 mmol) in acetonitrile (60 mL) was added 4-methylbenzenesulfonic anhydride (1.30 g, 3.98 mmol). The mixture was stirred at 35° C. for 8 hours. After completion, (2S,6R)-tert-butyl 2,6-dimethylpiperazine-1-carboxylate (1.02 g, 4.77 mmol) was added into the reaction solution. The reaction mixture was stirred at 35° C. overnight. After completion, the mixture was poured into ice-water (200 mL) and extracted with ethyl acetate (100 mL×3). Concentrated and the residue was purified by C18 column with 20-95% acetonitrile in water to afford tert-butyl (2S,6R)-4-(11-chloro-6-oxo-3-(thiophen-2-yl)-10-(trifluoromethyl)-3,4-dihydro-2H,6H-[1,4]thiazepino[2,3,4-ij]quinazolin-8-yl)-2,6-dimethylpiperazine-1-carboxylate (8) (720 mg, 74% yield) as a pale yellow solid. MS (ESI) m/z 615.2 [M+H]⁺.

### tert-butyl (2S,6R)-4-(11-(4-fluorophenyl)-6-oxo-3-(thiophen-2-yl)-10-(trifluoromethyl)-3,4-dihydro-2H,6H-[1,4]thiazepino[2,3,4-ij]quinazolin-8-yl)-2,6-dimethylpiperazine-1-carboxylate (9)

To a mixture of (2S,6R)-4-(11-chloro-6-oxo-3-(thiophen-2-yl)-10-(trifluoromethyl)-3,4-dihydro-2H,6H-[1,4]thiazepino[2,3,4-ij]quinazolin-8-yl)-2,6-dimethylpiperazine-1-carboxylate (8) (150 mg, 0.24 mmol) in 1,4-dioxane and water was added [2′-(Amino)[1,1′-biphenyl]-2-yl][[2′,6′-bis(1-methylethoxy)[1,1′-biphenyl]-2-yl]dicyclohexylphosphine]chloropalladium (38 mg, 0.048 mmol), potassium phosphate trihydrate (320 mg, 1.2 mmol) and (4-fluorophenyl)boronic acid (101 mg, 0.72 mmol) at 25° C. The mixture was stirred at 80° C. for 2 hours. After completion, the mixture was concentrated and the residue was purified by silica gel column chromatography (dichloromethane/methanol=50/1) to afford tert-butyl (2S,6R)-4-(11-(4-fluorophenyl)-6-oxo-3-(thiophen-2-yl)-10-(trifluoromethyl)-3,4-dihydro-2H,6H-[1,4]thiazepino[2,3,4-ij]quinazolin-8-yl)-2,6-dimethylpiperazine-1-carboxylate (9) (220 mg, crude) as a yellow solid. (ESI) m/z 675.2 [M+H]⁺.

### 8-((3S,5R)-3,5-dimethylpiperazin-1-yl)-11-(4-fluorophenyl)-3-(thiophen-2-yl)-10-(trifluoromethyl)-3,4-dihydro-2H,6H-[1,4]thiazepino[2,3,4-ij]quinazolin-6-one (10)

To a mixture of tert-butyl (2S,6R)-4-(11-(4-fluorophenyl)-6-oxo-3-(thiophen-2-yl)-10-(trifluoromethyl)-3,4-dihydro-2H,6H-[1,4]thiazepino[2,3,4-ij]quinazolin-8-yl)-2,6-dimethylpiperazine-1-carboxylate (9) (220 mg, crude) in dichloromethane (4 mL) was added trifluoroacetic acid (2 mL) at 25° C. The mixture was stirred at 25° C. for 1 hour. After completion, the mixture was concentrated and adjusted PH to 8 with ammonia in methanol at 0° C. The mixture was concentrated and purified by silica gel column chromatography (dichloromethane/methanol=20/1) to afford 8-((3S,5R)-3,5-dimethylpiperazin-1-yl)-11-(4-fluorophenyl)-3-(thiophen-2-yl)-10-(trifluoromethyl)-3,4-dihydro-2H,6H-[1,4]thiazepino[2,3,4-ij]quinazolin-6-one (10) (130 mg, crude) as yellow solid. MS (ESI) m/z 575.2 [M+H]⁺.

### 8-((3S,5R)-4-acryloyl-3,5-dimethylpiperazin-1-yl)-11-(4-fluorophenyl)-3-(thiophen-2-yl)-10-(trifluoromethyl)-3,4-dihydro-2H,6H-[1,4]thiazepino[2,3,4-ij]quinazolin-6-one (11)

To a mixture of 8-((3S,5R)-3,5-dimethylpiperazin-1-yl)-11-(4-fluorophenyl)-3-(thiophen-2-yl)-10-(trifluoromethyl)-3,4-dihydro-2H,6H-[1,4]thiazepino[2,3,4-ij]quinazolin-6-one (10) (130 mg, 0.23 mmol) in dichloromethane (6 mL) was added triethylamine (46 mg, 0.46 mmol) and acrylic anhydride (44 mg, 0.35 mmol) at 0° C. The mixture was stirred at 25° C. for 1 hour. After completion, the mixture was added methanol at 25° C. and concentrated. The mixture was purified by preparative high performance liquid chromatography (20% to 95% acetonitrile in water) to afford 8-((3S,5R)-4-acryloyl-3,5-dimethylpiperazin-1-yl)-11-(4-fluorophenyl)-3-(thiophen-2-yl)-10-(trifluoromethyl)-3,4-dihydro-2H,6H-[1,4]thiazepino[2,3,4-ij]quinazolin-6-one (11) (50 mg, 33% yield for three steps) as a pale yellow powder. MS (ESI) m/z 629.0 [M+H]⁺.

The above racemate (50 mg) was dissolved in ethanol (5 mL) and separated by chiral supercritical fluid chromatography (separation condition: Column: IC 5 μm 20×250 mm; Mobile Phase: MeOH/DCM=90/10 at 20 mL/min; Temp: 30° C.; Wavelength: 254 nm) to afford the title compounds CA-5578-F1 (18 mg, yield: 36%, 100% ee) and CA-5578-F2 (16 mg, yield: 32%, 99.8% ee); Chiral HPLC Analytical: on IC was using 5 μm 4.6×250 mm column, Mobile Phase: MeOH/DCM=90/10 at 1 mL/min; temperature: 30° C.; Wavelength: 254 nm).

### (R)-8-((3S,5R)-4-acryloyl-3,5-dimethylpiperazin-1-yl)-11-(4-fluorophenyl)-3-(thiophen-2-yl)-10-(trifluoromethyl)-3,4-dihydro-2H,6H-[1,4]thiazepino[2,3,4-ij]quinazolin-6-one (P1)

¹H NMR (400 MHz, CDCl₃) δ 8.09 (s, 1H), 7.28-7.27 (m, 1H), 7.21-7.15 (m, 4H), 7.00-6.99 (m, 1H), 6.97-6.95 (m, 1H), 6.63 (dd, J=16.4 Hz, 10.4 Hz, 1H), 6.41 (dd, J=16.8 Hz, 2.0 Hz, 1H), 5.78 (dd, J=10.4 Hz, 2.0 Hz, 1H), 4.87-4.84 (m, 1H), 4.76-4.53 (m, 3H), 4.21-4.15 (m, 2H), 4.11-4.03 (m, 1H), 3.71-3.61 (m, 1H), 3.41-3.32 (m, 2H), 3.13-3.02 (m, 1H), 1.60 (d, J=7.2 Hz, 3H), 1.52 (d, J=6.8 Hz, 3H); Chiral HPLC fraction 1: e.e. =100%, Rt=10.566 min.

### (S)-8-((3S,5R)-4-acryloyl-3,5-dimethylpiperazin-1-yl)-11-(4-fluorophenyl)-3-(thiophen-2-yl)-10-(trifluoromethyl)-3,4-dihydro-2H,6H-[1,4]thiazepino[2,3,4-ij]quinazolin-6-one (P2)

¹H NMR (400 MHz, CDCl₃) δ 8.09 (s, 1H), 7.31-7.29 (m, 1H), 7.20-7.17 (m, 4H), 7.00-6.99 (m, 1H), 6.97-6.95 (m, 1H), 6.63 (dd, J=16.8 Hz, 10.4 Hz, 1H), 6.41 (dd, J=16.8 Hz, 2.0 Hz, 1H), 5.78 (dd, J=10.4 Hz, 1.6 Hz, 1H), 4.94-4.83 (m, 1H), 4.78-4.54 (m, 3H), 4.21-4.15 (m, 2H), 4.11-4.02 (m, 1H), 3.72-3.60 (m, 1H), 3.41-3.33 (m, 2H), 3.10-3.03 (m, 1H), 1.60-1.57 (m, 3H), 1.52 (d, J=6.8 Hz, 3H); Chiral HPLC fraction 2: ee.=99.8%, Rt=12.223 min.

### Example 126: (S)-8-((3S,5R)-4-acryloyl-3,5-dimethylpiperazin-1-yl)-11-(4-fluorophenyl)-3-(5-fluoropyridin-3-yl)-10-(trifluoromethyl)-3,4-dihydro-2H,6H-[1,4]thiazepino[2,3,4-ij]quinazolin-6-one

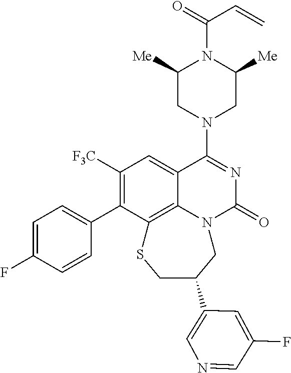

### Step 1: tert-butyl (2S,6R)-4-((R)-11-chloro-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate and tert-butyl (2S,6R)-4-((S)-11-chloro-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate

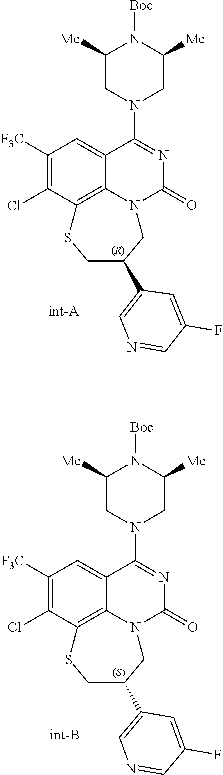

The title compounds were prepared analogously to Example 104, where pyridin-3-ylboronic acid was replaced with (5-fluoropyridin-3-yl)boronic acid in step 1. The title compounds were isolated as a 1:1 mixture of diastereomers. tert-Butyl (2S,6R)-4-((R)-11-chloro-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate and tert-butyl (2S,6R)-4-((S)-11-chloro-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate were obtained as single diastereomers by purification of the mixture by SFC (column=Daicel Chiralpak IC 250 mm×30 mm, 10 um; phase A: ethanol, phase B: CO2; gradient: 65% A in B for 7.45 minutes runtime) and characterized by SFC (Column: Chiralpak IC-3, 50×4.6 mm, I.D.=3 um, stabilized at 35° C. with a back pressure of 100 bar. Mobile phase: 60% ethanol containing 0.05% diethylamine in CO2, flow rate=3 mL/min. Detector: Photodiode-array). Int-a: SFC Rt=1.58 min; MS (ESI) m/z: 628.2 [M+H]+. Int-b: SFC Rt=2.87 min; MS (ESI) m/z: 628.2 [M+H]+.

### Step 2: tert-butyl (2S,6R)-4-((S)-11-(4-fluorophenyl)-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate

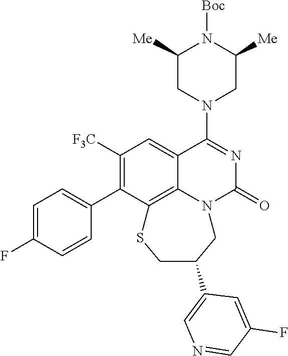

The title compound was prepared analogously to Example 100, step 9, where tert-butyl (2S,6R)-4-((S)-11-chloro-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((S)-11-chloro-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate. The title compound was isolated in 36% yield as a yellow solid. MS (ESI) m/z: 688.2 [M+H]+.

### Step 3: (S)-8-((3S,5R)-3,5-dimethylpiperazin-1-yl)-11-(4-fluorophenyl)-3-(5-fluoropyridin-3-yl)-10-(trifluoromethyl)-3,4-dihydro-2H,6H-[1,4]thiazepino[2,3,4-ij]quinazolin-6-one

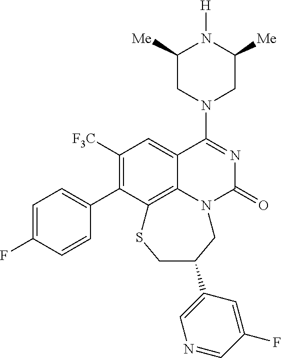

The title compound was prepared analogously to Example 100, step 10, where tert-butyl (2S,6R)-4-((3S)-11-(5-chloro-2,4-difluorophenyl)-6-oxo-3-(pyridin-4-yl)-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((S)-11-(4-fluorophenyl)-3-(5-fluoropyridin-3-yl)-6-oxo-10-(trifluoromethyl)-3,4-dihydro-2H,6H-[1,4]thiazepino[2,3,4-ij]quinazolin-8-yl)-2,6-dimethylpiperazine-1-carboxylate. The title compound was isolated in 99% yield as a yellow solid. MS (ESI) m/z: 588.2 [M+H]+.

### Step 4: (S)-8-((3S,5R)-4-acryloyl-3,5-dimethylpiperazin-1-yl)-11-(4-fluorophenyl)-3-(5-fluoropyridin-3-yl)-10-(trifluoromethyl)-3,4-dihydro-2H,6H-[1,4]thiazepino[2,3,4-ij]quinazolin-6-one

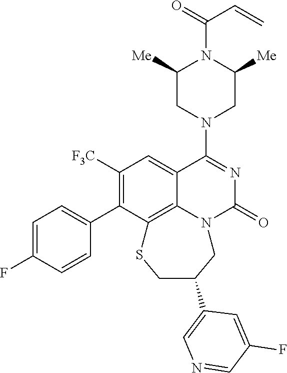

The title compound was prepared analogously to Example 100, step 11, where (3S)-11-(5-chloro-2,4-difluorophenyl)-8-((3S,5R)-3,5-dimethylpiperazin-1-yl)-3-(pyridin-4-yl)-10-(trifluoromethyl)-3,4-dihydro-2H,6H-[1,4]thiazepino[2,3,4-ij]quinazolin-6-one was replaced with (S)-8-((3S,5R)-3,5-dimethylpiperazin-1-yl)-11-(4-fluorophenyl)-3-(5-fluoropyridin-3-yl)-10-(trifluoromethyl)-3,4-dihydro-2H,6H-[1,4]thiazepino[2,3,4-ij]quinazolin-6-one. The title compound was isolated in 84% yield as a yellow solid. MS (ESI) m/z: 642.2 [M+1]+.

1H-NMR (400 MHz, CDCl3)=8.47 (s, 1H), 8.42 (s, 1H), 8.13 (S, 1H), 7.46 (m, i1H), 7.25-7.19 (m, 4H), 6.61 (dd, J=16 Hz, J=6 Hz, 1H), 6.44 (d, J=16 Hz, 1H), 5.80 (d, J=6 Hz, 1H), 4.80 (m, 4H), 4.21 (m, 2H), 3.75 (m, 2H), 3.40 (m, 2H), 3.06 (m, 1H), 1.57 (m, 6H).

It will be appreciated by those skilled in the art that the compounds above possess chiral centers as well potential axial asymmetry, i.e., atropisomers. Each of the compounds may be provided as a mixture of diastereomers or in any diasteromerically pure form.

Additional embodiments are provided herein as follows:

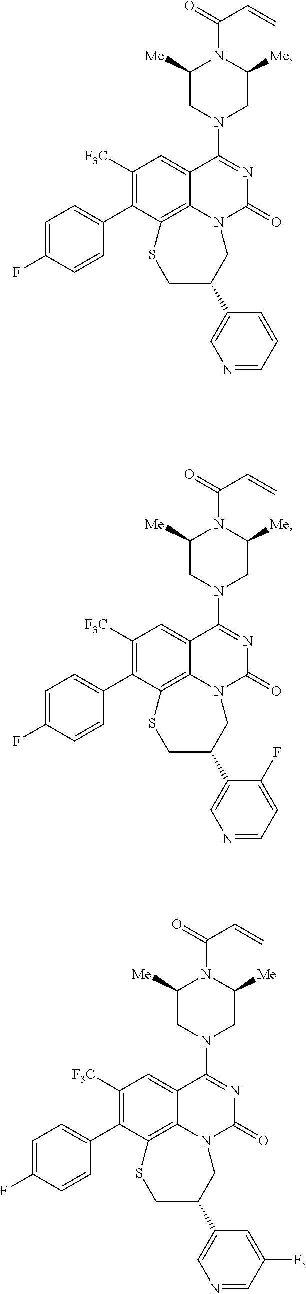 [SMILES: C=CC(=O)N1[C@H](C)CN(c2nc(=O)n3c4c(c(-c5ccc(F)cc5)c(C(F)(F)F)cc24)SC[C@@H](c2cccnc2)C3)C[C@@H]1C ; C=CC(=O)N1[C@H](C)CN(c2nc(=O)n3c4c(c(-c5ccc(F)cc5)c(C(F)(F)F)cc24)SC[C@@H](c2cnccc2F)C3)C[C@@H]1C ; C=CC(=O)N1[C@H](C)CN(c2nc(=O)n3c4c(c(-c5ccc(F)cc5)c(C(F)(F)F)cc24)SC[C@@H](c2cncc(F)c2)C3)C[C@@H]1C]

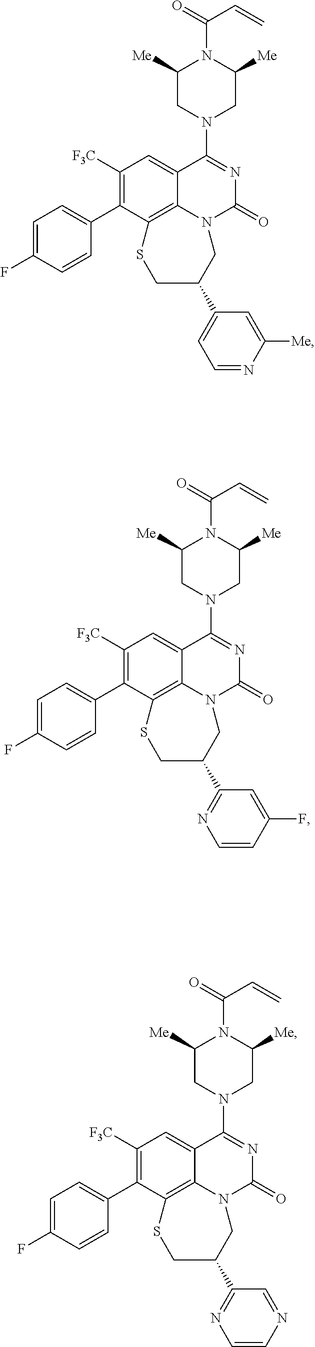 [SMILES: C=CC(=O)N1[C@H](C)CN(c2nc(=O)n3c4c(c(-c5ccc(F)cc5)c(C(F)(F)F)cc24)SC[C@@H](c2ccnc(C)c2)C3)C[C@@H]1C ; C=CC(=O)N1[C@H](C)CN(c2nc(=O)n3c4c(c(-c5ccc(F)cc5)c(C(F)(F)F)cc24)SC[C@@H](c2cc(F)ccn2)C3)C[C@@H]1C ; C=CC(=O)N1[C@H](C)CN(c2nc(=O)n3c4c(c(-c5ccc(F)cc5)c(C(F)(F)F)cc24)SC[C@@H](c2cnccn2)C3)C[C@@H]1C]

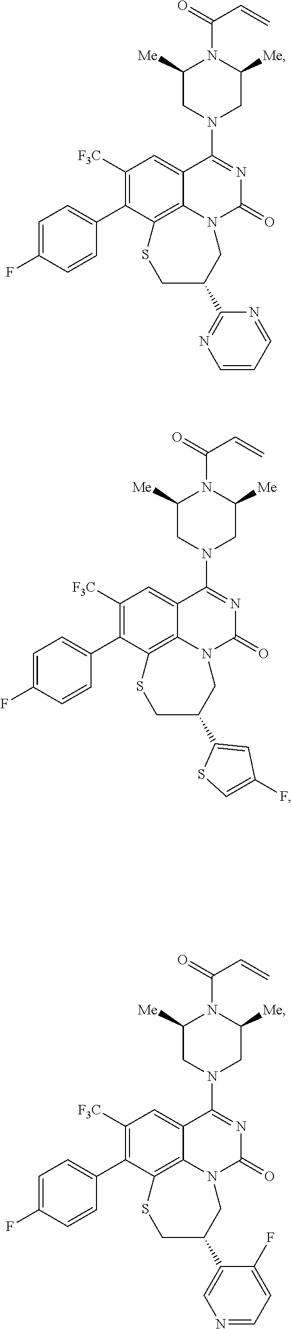 [SMILES: C=CC(=O)N1[C@H](C)CN(c2nc(=O)n3c4c(c(-c5ccc(F)cc5)c(C(F)(F)F)cc24)SC[C@@H](c2ncccn2)C3)C[C@@H]1C ; C=CC(=O)N1[C@H](C)CN(c2nc(=O)n3c4c(c(-c5ccc(F)cc5)c(C(F)(F)F)cc24)SC[C@@H](c2cc(F)cs2)C3)C[C@@H]1C ; C=CC(=O)N1[C@H](C)CN(c2nc(=O)n3c4c(c(-c5ccc(F)cc5)c(C(F)(F)F)cc24)SC[C@@H](c2cnccc2F)C3)C[C@@H]1C]

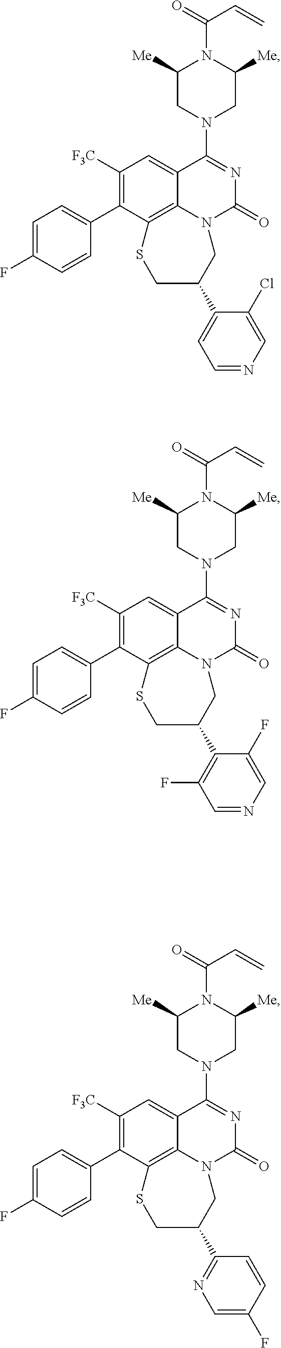 [SMILES: C=CC(=O)N1[C@H](C)CN(c2nc(=O)n3c4c(c(-c5ccc(F)cc5)c(C(F)(F)F)cc24)SC[C@@H](c2ccncc2Cl)C3)C[C@@H]1C ; C=CC(=O)N1[C@H](C)CN(c2nc(=O)n3c4c(c(-c5ccc(F)cc5)c(C(F)(F)F)cc24)SC[C@@H](c2c(F)cncc2F)C3)C[C@@H]1C ; C=CC(=O)N1[C@H](C)CN(c2nc(=O)n3c4c(c(-c5ccc(F)cc5)c(C(F)(F)F)cc24)SC[C@@H](c2ccc(F)cn2)C3)C[C@@H]1C]

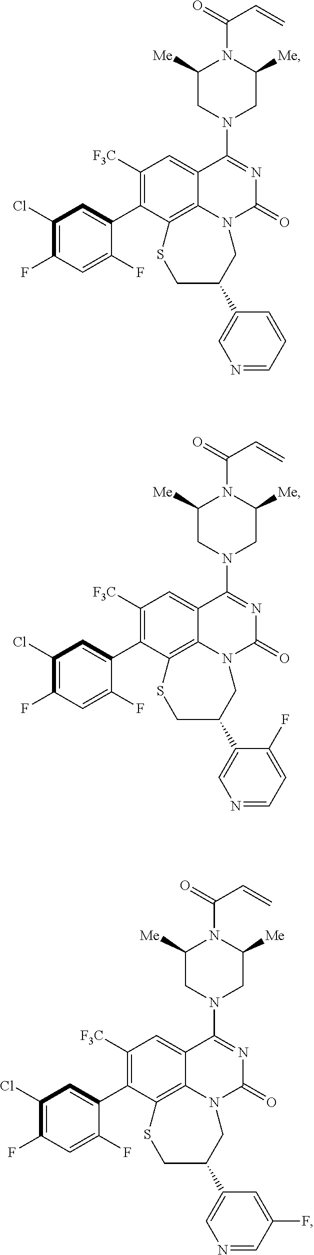

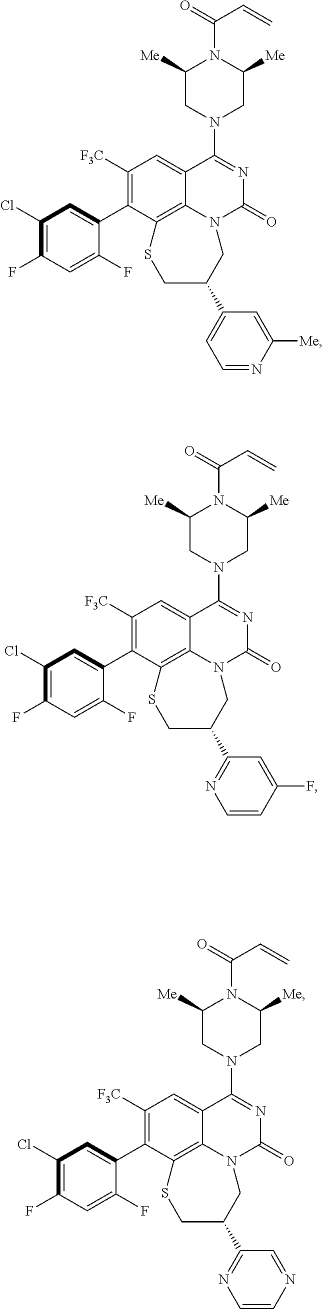

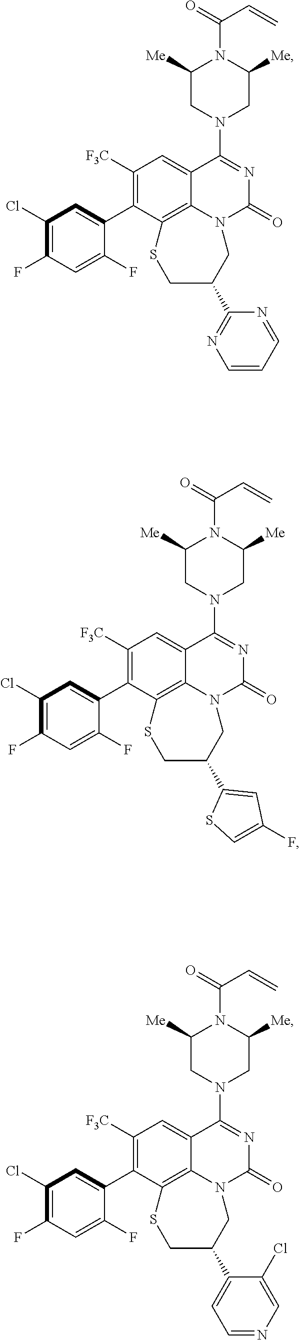

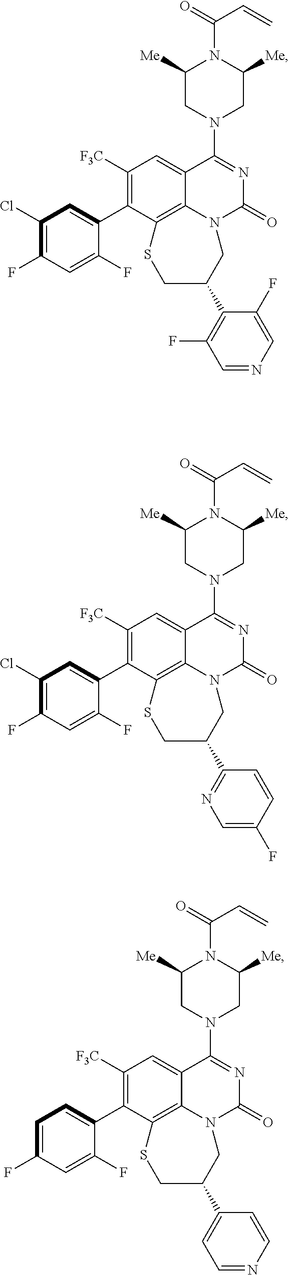

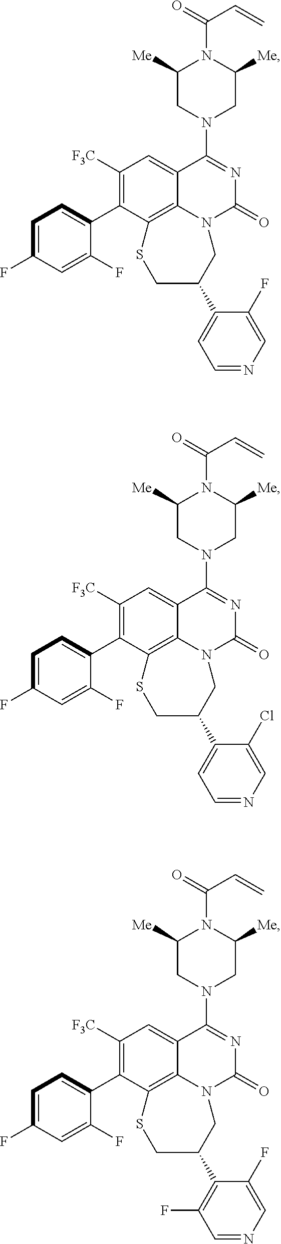

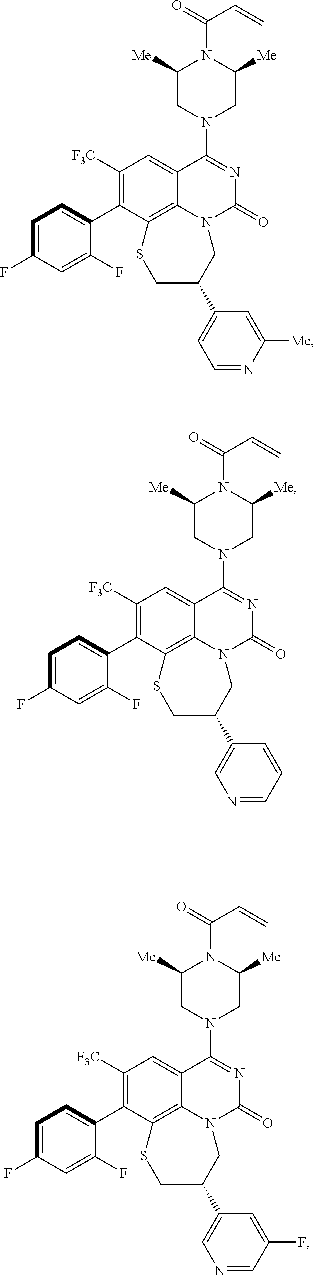

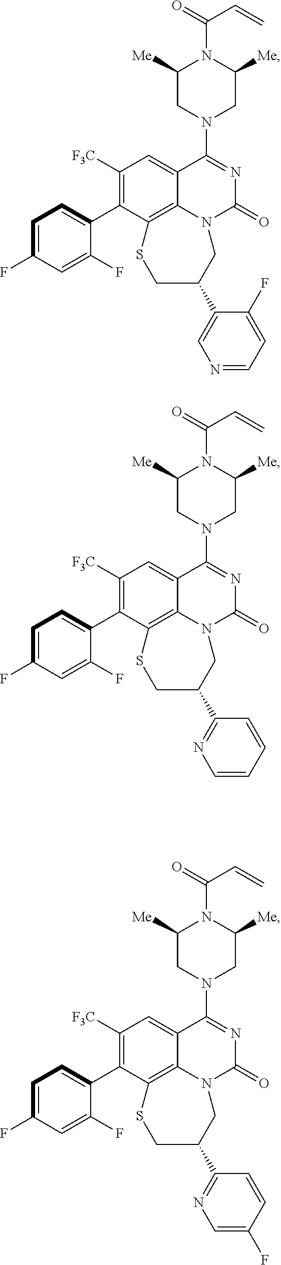

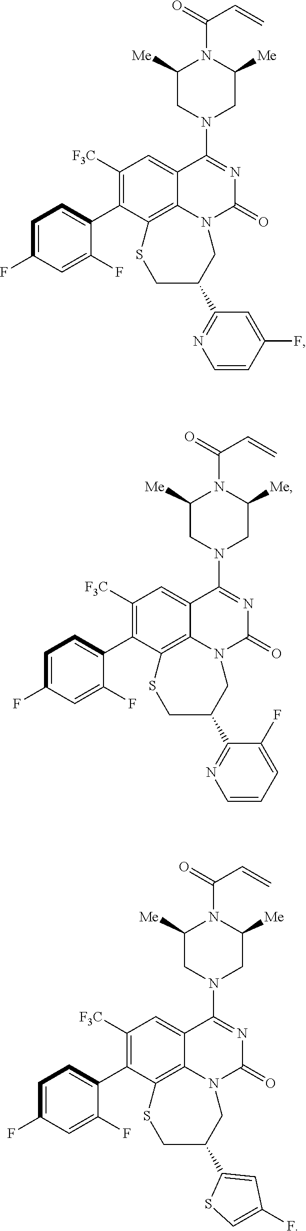

### Inhibition of KRAS G12C-Mediates Phospho-ERK1/2 Inhibition by Compounds of Formula I

This Example illustrates that various compounds of the present disclosure inhibit KRAS G12C thereby mediating the downstream inhibition of phospho-ERK1/2.

KRAS G12C mutant cell lines, NCI H358 (ATCC, CRL-5807), and Ras Initiative (R1) KRAS G12C were cultured according to published protocols and maintained at 37° C. in 5% CO2. The phospho-ERK HTRF assay was executed following provider's protocol (CisBio #64AERPEH). NCI-H358 or RI KRAS G12C cells were plated at a density of 50,000 cells per well in a 96-well plate (Corning #3903) in respective medias (for NCI-H358, RPMI+10% FBS+1% Pen/Strep, and for RI KRAS G12C, DMEM+10% FBS+1% Pen/Strep+4 ug/ml Blasticidin) and maintained at 37° C. in 5% CO₂. Cells were allowed to adhere overnight and treated the following day with a Tecan D300e Digital Dispenser (Tecan Group Ltd., Switzerland) using an 11-point dose response starting at 2,500 nM of exemplified compounds followed by sequential 1:3 dilutions for either 4 hours or 16 hours. Following compound treatment, the cells were washed once with ice-cold PBS. Cells were lysed by adding 50 μl of lysis buffer (1×) supplemented with 1× Pierce Halt Protease and Phosphatase inhibitor and incubated for 30 minutes at 4° C. with shaking. After lysis, 16 μL of cell lysate from the 96-well cell-culture plate was transferred to a 384-well plate (Perkin Elmer #6007290). The premixed antibody solution was prepared by mixing (vol/vol) advanced phospho-ERK1/2 d2 antibody and advanced phospho-ERK1/2 Eu Cryptate antibody. The premixed antibody solution (4 L) was added to the detection plate containing cell lysate. The detection plate was incubated overnight at 4° C., the HTRF signal was read the next day by using either a Spectramax M5 or Spectramax i3 microplate reader (Molecular Devices, San Jose, CA, USA), and data was processed according to manufacturer's protocol.

Compounds prepared according to the preceding procedures and characterized for inhibition of p-ERK according to the protocol provided herein are summarized in Table 3:

TABLE 3

| | | MS | | pERK |
|---|---|---|---|---|
| Ex. | | (ESI) | | (RI) |
| No. | Structure | [M + H] | ¹H NMR | nM |
| 100A | 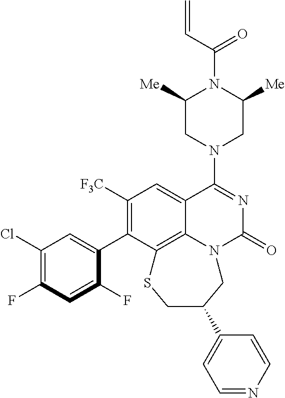 | 676 | ¹H NMR (400 MHz, CDCl₃) δ ppm 1.51 − 1.59 (m, 6 H) 3.15 (br d, J = 13.6 Hz, 1 H) 3.41 (td, J = 12.8, 4.8 Hz, 2 H) 3.70 − 3.87 (m, 2 H) 4.12 − 4.27 (m, 2 H) 4.53 − 4.91 (m, 4 H) 5.80 (dd, J = 10.4, 2.0 Hz, 1 H) 6.43 (dd, J = 16.8, 2.0 Hz, 1 H) 6.64 (dd, J = 16.4, 10.4 Hz, 1 H) 7.06 (t, J = 8.8 Hz, 1 H) 7.28 − 7.34 (m, 3 H) 8.14 (s, 1 H) 8.49 − 8.67 (m, 2 H) | +++ |
| 100B | 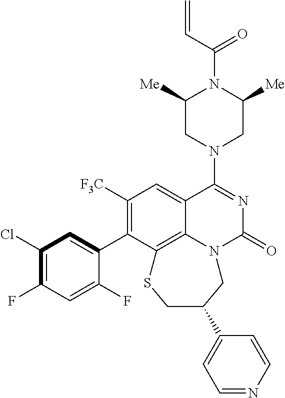 | 676 | ¹H NMR (400 MHz, CDCl₃) δ ppm 1.53 (br d, J = 6.8Hz, 6 H) 3.06 − 3.27 (m, 1 H) 3.41 (ddd, J = 17.6, 13.2, 4.4 Hz, 2 H) 3.61 − 3.83 (m, 2 H) 4.14 − 4.23 (m, 2 H) 4.53 − 4.90 (m, 4 H) 5.80 (dd, J = 10.4, 2.0 Hz, 1 H) 6.43 (dd, J = 16.8, 1.6 Hz, 1 H) 6.64 (dd, J = 16.8, 10.4 Hz, 1 H) 7.10 (t, J = 8.8 Hz, 1 H) 7.23 − 7.27 (m, 1 H) 7.30 (br d, J = 5.2 Hz, 2 H) 8.15 (s, 1 H) 8.59 (d, J = 6.0 Hz, 2 H) | ++++ |
| 101A | 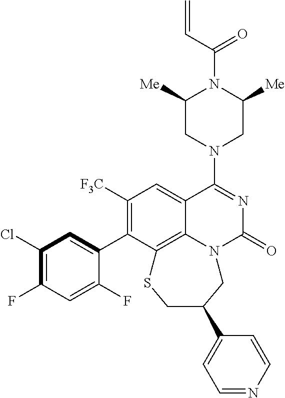 | 676 | ¹H NMR (400 MHz, CDCl₃) δ ppm = 1.50 − 1.61 (m, 6 H) 3.16 (br d, J = 12.8 Hz, 1 H) 3.41 (td, J = 12.4, 4.8 Hz, 2 H) 3.67 − 3.95 (m, 2 H) 4.12 − 4.25 (m, 2 H) 4.42 − 5.05 (m, 4 H) 5.72 − 5.90 (m, 1 H) 6.43 (dd, J = 16.8, 1.6 Hz, 1 H) 6.56 − 6.72 (m, 1 H) 7.06 (t, J = 8.8 Hz, 1 H) 7.31 (t, J = 7.6 Hz, 1 H) 7.38 (d, J = 6.0 Hz, 2 H) 8.14 (s, 1 H) 8.62 (d, J = 6.0 Hz, 2 H) | +++ |
| 101B | 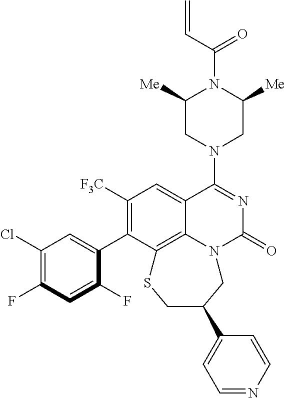 | 676 | ¹H NMR (400 MHz, CDCl₃) δ ppm = 1.48 − 1.61 (m, 6 H) 2.99 − 3.24 (m, 1 H) 3.41 (ddd, J = 17.6, 13.2, 4.6 Hz, 2 H) 3.57 − 3.89 (m, 2 H) 4.19 (br dd, J = 13.2, 5.6 Hz, 2 H) 4.46 − 5.00 (m, 4 H) 5.80 (dd, J = 10.4, 1.6 Hz, 1 H) 6.43 (dd, J = 16.8, 1.6 Hz, 1 H) 6.64 (dd, J = 16.8, 10.4 Hz, 1 H) 7.10 (t, J = 8.8 Hz, 1 H) 7.25 (s, 1 H) 7.31 (br d, J = 5.6 Hz, 2 H) 8.15 (s, 1 H) 8.52 − 8.67 (m, 2 H) | ++ |
| 102 | 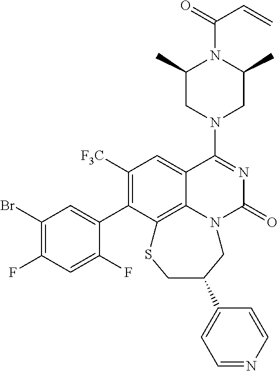 | 722.1 | ¹H NMR (400 MHz, CDCl₃) δ ppm 1.54 (br d, J = 6.8 Hz, 6 H) 3.04 − 3.25 (m, 1 H) 3.34 − 3.46 (m, 2 H) 3.57 − 3.89 (m, 2 H) 4.19 (br dd, J = 13.6, 6.8 Hz, 2 H) 4.50 − 4.93 (m, 4 H) 5.80 (dd, J = 10.4, 1.6 Hz, 1 H) 6.38 − 6.49 (m, 1 H) 6.56 − 6.71 (m, 1 H) 6.99 − 7.15 (m, 1 H) 7.31 (br dd, J = 4.4, 2.0 Hz, 2 H) 7.43 (dt, J = 14.4, 7.2 Hz, 1 H) 8.14 (d, J = 3.6 Hz, 1 H) 8.51 − 8.68 (m, 2 H) | ++++ |
| 103 | 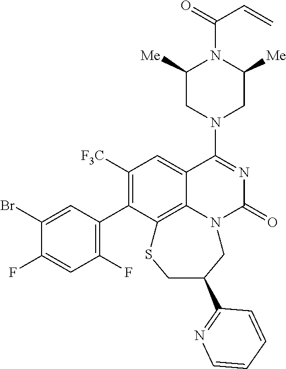 | 722.1 | ¹H NMR (400 MHz, DMSO-d₆) δ, 8.58 − 8.48 (m, 1H), 8.09 (s, 1H), 7.88 − 7.72 (m, 3H), 7.43 (d, J = 8.0 Hz, 1H), 7.28 (br dd, J = 5.2, 7.6 Hz, 1H), 6.83 (dd, J = 10.4, 16.4 Hz, 1H), 6.21 (dd, J = 2.4, 16.8 Hz, 1H), 5.78 − 5.73 (m, 1H), 4.87 − 4.75 (m, 2H), 4.67 − 4.53 (m, 2H), 4.13 − 4.05 (m, 2H), 3.96 − 3.80 (m, 1H), 3.80 − 3.68 (m, 2H), 3.29 − 3.23 (m, 2H), 1.46 − 1.39 (m, 6H) | + |
| 104A | 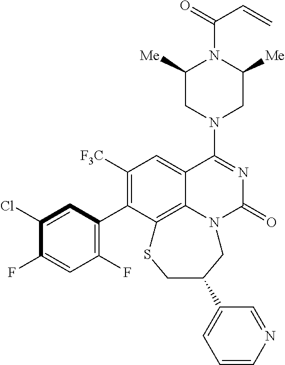 | 676 | ¹H NMR (400 MHz, CDCl₃) δ 8.87 − 8.39 (m, 2H), 8.15 (s, 1H), 7.74 (d, J = 6.8 Hz, 1H), 7.40 − 7.28 (m, 2H), 7.10 (t, J = 8.8 Hz, 1H), 6.64 (dd, J = 10.4, 16.4 Hz, 1H), 6.43 (dd, J = 2.0, 16.4 Hz, 1H), 5.83 − 5.76 (m, 1H), 5.11 − 4.48 (m, 4H), 4.19 (d, J = 13.2 Hz, 2H), 3.89 − 3.58 (m, 2H), 3.46 − 3.36 (m, 2H), 3.27 − 3.00 (m, 1H), 1.61 − 1.53 (m, 6H) | ++++ |
| 104B | 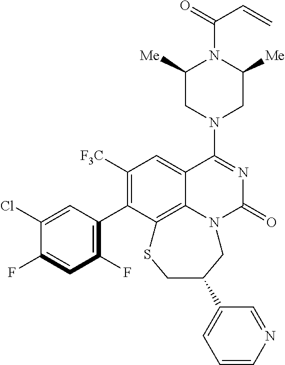 | 676 | ¹H NMR (400 MHz, CDCl₃) δ 8.91 − 8.30 (m, 2H), 8.14 (s, 1H), 7.75 (d, J = 7.6 Hz, 1H), 7.32 (t, J = 7.6 Hz, 2H), 7.10 − 7.02 (m, 1H), 6.70 − 6.58 (m, 1H), 6.47 − 6.39 (m, 1H), 5.83 − 5.76 (m, 1H), 4.91 − 4.54 (m, 4H), 4.23 − 4.15 (m, 2H), 3.91 − 3.72 (m, 2H), 3.40 (dt, J = 4.4, 13.2 Hz, 2H), 3.22 − 3.03 (m, 1H), 1.60 (d, J = 6.8 Hz, 3H), 1.54 (d, J = 6.8 Hz, 3H) | +++ |
| 105A | 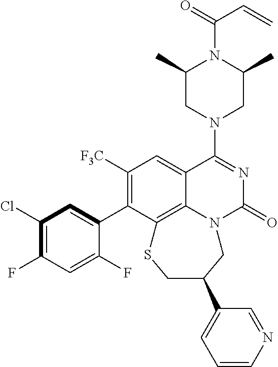 | 676 | ¹H NMR (400 MHz, DMSO-d₆) δ 8.59 (d, J = 1.6 Hz, 1H), 8.49 (dd, J = 1.2, 4.8 Hz, 1H), 8.10 (s, 1H), 7.88 − 7.68 (m, 3H), 7.38 (dd, J = 5.2, 7.6 Hz, 1H), 6.82 (dd, J = 10.4, 16.4 Hz, 1H), 6.20 (dd, J = 2.4, 16.4 Hz, 1H), 5.80 − 5.70 (m, 1H), 4.84 − 4.72 (m, 1H), 4.71 − 4.42 (m, 3H), 4.09 (d, J = 13.2 Hz, 2H), 4.03 − 3.73 (m, 1H), 3.72 − 3.32 (m, 2H), 3.28 − 3.21 (m, 2H), 1.48 − 1.32 (m, 6H) | ++ |
| 105B | 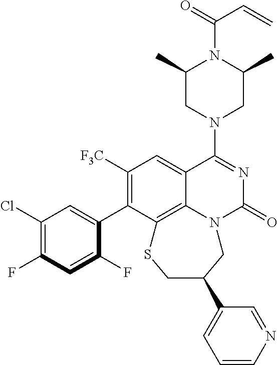 | 676 | ¹H NMR (400 MHz, DMSO-d₆) δ 8.56 (s, 1H), 8.46 (d, J = 4.4 Hz, 1H), 8.09 (s, 1H), 7.89 − 7.62 (m, 3H), 7.42 − 7.26 (m, 1H), 6.82 (dd, J = 10.4, 16.4 Hz, 1H), 6.20 (dd, J = 2.4, 16.4 Hz, 1H), 5.81 − 5.67 (m, 1H), 4.98 − 4.38 (m, 4H), 4.09 (d, J = 13.2 Hz, 2H), 3.98 − 3.35 (m, 3H), 3.26 (s, 2H), 1.54 − 1.29 (m, 6H) | + |
| 106 | 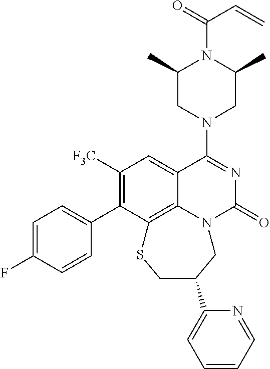 | 624.3 | ¹H NMR (400 MHz, CDCl₃) δ, 8.46 (br d, J = 4.4 Hz, 1H), 8.03 (s, 1H), 7.59 (dt, J = 1.6, 7.6 Hz, 1H), 7.23 (d, J = 7.6 Hz, 1H), 7.17 − 7.06 (m, 5H), 6.57 (dd, J = 10.4, 16.8 Hz, 1H), 6.35 (dd, J = 1.6, 16.8 Hz, 1H), 5.76 − 5.65 (m, 1H), 4.92 (br d, J = 4.4 Hz, 2H), 4.76 − 4.39 (m, 2H), 4.15 − 4.07 (m, 2H), 3.92 − 3.75 (m, 1H), 3.59 − 3.40 (m, 2H), 3.32 − 3.23 (m, 2H), 1.46 (br d, J = 6.8 Hz, 6H) | ++++ |
| 107A | 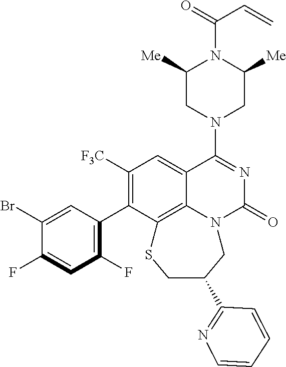 | 720 | ¹H NMR (400 MHz, CDCl₃) δ, 8.57 (br d, J = 4.4 Hz, 1H), 8.12 (s, 1H), 7.73 − 7.64 (m, 1H), 7.42 (t, J = 7.2 Hz, 1H), 7.36 − 7.30 (m, 1H), 7.23 − 7.16 (m, 1H), 7.06 − 6.97 (m, 1H), 6.64 (dd, J = 10.4, 16.8 Hz, 1H), 6.43 (dd, J = 2.0, 16.8 Hz, 1H), 5.79 (dd, J = 2.0, 10.4 Hz, 1H), 5.08 − 4.93 (m, 2H), 4.84 − 4.49 (m, 2H), 4.22 − 4.13 (m, 2H), 4.01 − 3.91 (m, 1H), 3.71 − 3.52 (m, 2H), 3.41 − 3.32 (m, 2H), 1.55 (br d, J = 7.2 Hz, 6H) | +++ |
| 107B | 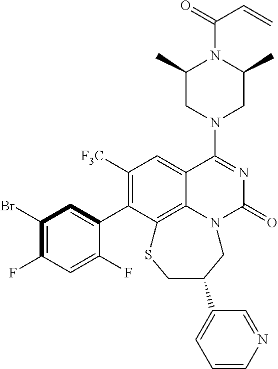 | 720 | ¹H NMR (400 MHz, CDCl₃) δ, 8.47 (br s, 1H), 8.11 − 8.01 (m, 1H), 7.67 − 7.55 (m, 1H), 7.35 (br t, J = 7.2 Hz, 1H), 7.26 (br d, J = 7.2 Hz, 1H), 7.15 − 7.10 (m, 1H), 6.95 (t, J = 8.8 Hz, 1H), 6.56 (dd, J = 10.4, 16.8 Hz, 1H), 6.35 (dd, J = 2.0, 16.8 Hz, 1H), 5.74 − 5.68 (m, 1H), 4.92 (br d, J = 4.4 Hz, 2H), 4.74 − 4.23 (m, 2H), 4.09 (br d, J = 12.8 Hz, 2H), 3.95 − 3.76 (m, 1H), 3.70 − 3.35 (m, 2H), 3.34 − 3.21 (m, 2H), 1.49 (br s, 6H) | ++++ |
| 108A | 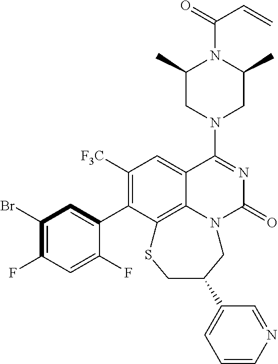 | 720 | ¹H NMR (400 MHz, CDCl₃) δ 9.15 − 8.23 (m, 1H), 8.15 (s, 1H), 7.75 (d, J = 6.8 Hz, 1H), 7.42 (t, J = 7.2 Hz, 1H), 7.37 − 7.27 (m, 2H), 7.08 (t, J = 8.4 Hz, 1H), 6.70 − 6.58 (m, 1H), 6.43 (dd, J = 2.0, 16.4 Hz, 1H), 5.80 (dd, J = 2.0, 10.4 Hz, 1H), 5.09 − 4.31 (m, 4H), 4.19 (d, J = 12.8 Hz, 2H), 3.97 − 3.54 (m, 2H), 3.46 − 3.34 (m, 2H), 3.29 − 2.97 (m, 1H), 1.60 (d, J = 6.8 Hz, 3H), 1.53 (d, J = 6.8 Hz, 3H) | ++++ |
| 108B | 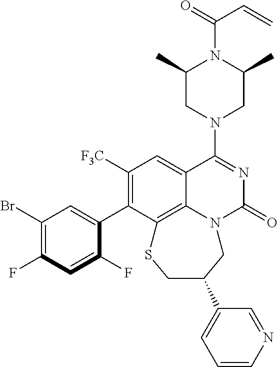 | 720 | ¹H NMR (400 MHz, CDCl₃) δ 9.10 − 8.29 (m, 1H), 8.14 (s, 1H), 7.84 − 7.73 (m, 1H), 7.46 (t, J = 7.0 Hz, 1H), 7.38 − 7.27 (m, 2H), 7.04 (t, J = 8.4 Hz, 1H), 6.64 (dd, J = 10.4, 16.4 Hz, 1H), 6.48 − 6.38 (m, 1H), 5.80 (dd, J = 1.6, 10.4 Hz, 1H), 5.07 − 4.42 (m, 4H), 4.19 (dd, J = 5.2, 13.6 Hz, 2H), 3.98 − 3.71 (m, 2H), 3.45 − 3.36 (m, 2H), 3.17 − 3.09 (m, 1H), 1.59 (d, J = 6.8 Hz, 3H), 1.54 (d, J = 6.8 Hz, 3H) | +++ |
| 109 | 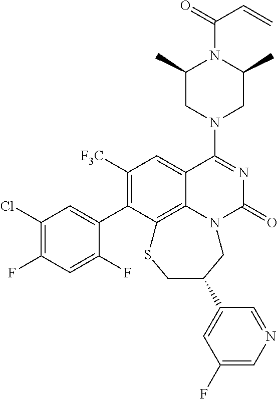 | 694.4 | ¹H NMR (400 MHz, CDCl₃) δ 8.48 (s, 1H), 8.43 (d, J = 2.4 Hz, 1H), 8.14 (s, 1H), 7.48 (d, J = 9.2 Hz, 1H), 7.36 − 7.27 (m, 1H), 7.14 − 7.03 (m, 1H), 6.68 − 6.60 (m, 1H), 6.47 − 6.40 (m, 1H), 5.80 (d, J = 11.6 Hz, 1H), 4.97 − 4.54 (m, 4H), 4.19 (dd, J = 5.2, 12.0 Hz, 2H), 3.96 − 3.67 (m, 2H), 3.41 (t, J = 4.4, 12.4 Hz, 2H), 3.20 − 3.04 (m, 1H), 1.59 (d, J = 6.8 Hz, 3H), 1.54 (d, J = 6.8 Hz, 3H) | +++ |
| 110 | 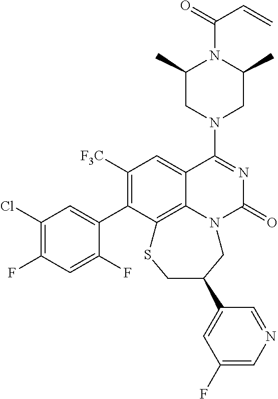 | 694 | ¹H NMR (400 MHz, CDCl₃) δ 8.48 (s, 1H), 8.43 (d, J = 2.4 Hz, 1H), 8.19 − 8.12 (m, 1H), 7.48 (d, J = 9.6 Hz, 1H), 7.35 − 7.27 (m, 1H), 7.15 − 7.02 (m, 1H), 6.70 − 6.59 (m, 1H), 6.43 (dd, J = 2.0, 16.4 Hz, 1H), 5.86 − 5.75 (m, 1H), 4.99 − 4.48 (m, 4H), 4.24 − 4.14 (m, 2H), 3.97 − 3.67 (m, 2H), 3.47 − 3.37 (m, 2H), 3.21 − 3.00 (m, 1H), 1.59 (d, J = 6.8 Hz, 3H), 1.54 (d, J = 6.8 Hz, 3H) | ++ |
| 111 | 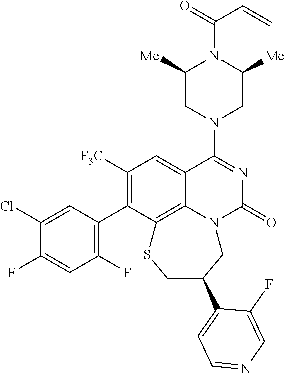 | 694 | ¹H NMR (400 MHz, CDCl₃) δ ppm 1.50 − 1.62 (m, 6 H) 2.97 − 3.27 (m, 1 H) 3.36 − 3.49 (m, 2 H) 3.56 − 3.88 (m, 1 H) 4.18 (d, J = 10.8 Hz, 3 H) 4.45 − 5.06 (m, 4 H) 5.80 (dd, J = 10.4, 2.0 Hz, 1 H) 6.37 − 6.47 (m, 1 H) 6.55 − 6.71 (m, 1 H) 7.07 (dt, J = 14.8, 8.8 Hz, 1 H) 7.22 − 7.27 (m, 1 H) 7.29 − 7.34 (m, 1 H) 8.14 (s, 1 H) 8.36 − 8.53 (m, 2 H) | ++ |
| 112A | 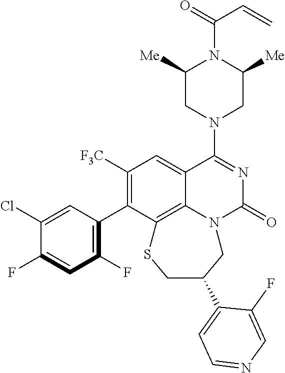 | 694.1 | ¹H NMR (400 MHz, CDCl₃) δ ppm 1.54 (d, J = 6.8 Hz, 3 H) 1.59 (d, J = 6.8 Hz, 3 H) 3.02 − 3.23 (m, 1 H) 3.42 (td, J = 12.4, 4.4 Hz, 2 H) 3.65 − 3.86 (m, 1 H) 4.11 − 4.24 (m, 3 H) 4.54 − 5.01 (m, 4 H) 5.74 − 5.84 (m, 1 H) 6.38 − 6.48 (m, 1 H) 6.58 − 6.70 (m, 1 H) 7.05 (t, J = 8.8 Hz, 1 H) 7.30 (t, J = 7.6 Hz, 2 H) 8.14 (s, 1 H) 8.32 − 8.61 (m, 2 H). | +++ |
| 112B | 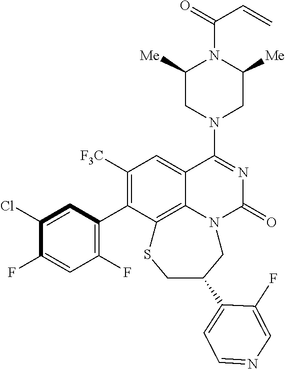 | 694.1 | ¹H NMR (400 MHz, CDCl₃) δ ppm 1.53 (d, J = 6.4 Hz, 3 H) 1.59 (br s, 3 H) 2.97 − 3.27 (m, 1 H) 3.36 − 3.46 (m, 2 H) 3.54 − 3.83 (m, 1 H) 4.05 − 4.26 (m, 3 H) 4.49 − 5.05 (m, 4 H) 5.80 (dd, J = 10.4, 2.0 Hz, 1 H) 6.43 (dd, J = 16.8, 2.0 Hz, 1 H) 6.64 (dd, J = 16.8, 10.4 Hz, 1 H) 7.09 (t, J = 8.8 Hz, 1 H) 7.25 (br s, 1 H) 7.29 (br s, 1 H) 8.14 (s, 1 H), 8.44 (d, 4.8 Hz), 8.47 (s, 1H) | ++++ |
| 113 | 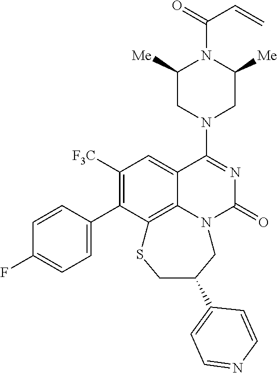 | 624.2 | ¹H NMR (400 MHz, CDCl₃) δ ppm 1.53 (d, J = 7.2 Hz, 3 H) 1.61 (d, J = 6.8 Hz, 3 H) 3.01 − 3.18 (m, 1 H) 3.41 (ddd, J = 17.2, 13.2, 4.0 Hz, 2 H) 3.60 − 3.83 (m, 2 H) 4.20 (dd, J = 12.8, 6.4 Hz, 2 H) 4.56 − 4.95 (m, 4 H) 5.74 − 5.85 (m, 1 H) 6.43 (dd, J = 16.8, 2.0 Hz, 1 H) 6.59 − 6.71 (m, 1 H) 7.17 − 7.26 (m, 4 H) 7.29 − 7.38 (m, 2 H) 8.13 (s, 1 H) 8.49 − 8.71 (m, 2 H) | ++++ |
| 114 | 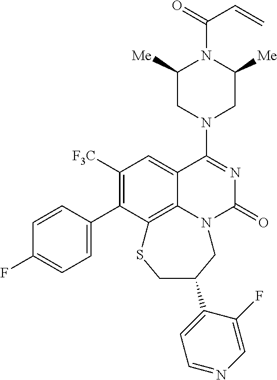 | 642.2 | ¹H NMR (400 MHz, CDCl₃) δ ppm 1.53 (d, J = 6.8 Hz, 3 H) 1.60 (d, J = 6.0 Hz, 3 H) 2.91 − 3.20 (m, 1 H) 3.34 − 3.48 (m, 2 H) 3.53 − 3.81 (m, 1 H) 3.97 − 4.26 (m, 3 H) 4.45 − 5.07 (m, 4 H) 5.73 − 5.85 (m, 1 H) 6.43 (dd, J = 16.8, 2.0 Hz, 1 H) 6.58 − 6.69 (m, 1 H) 7.14 − 7.23 (m, 3 H) 7.25 (br s, 1 H) 7.30 (br s, 1 H) 8.12 (s, 1 H) 8.29 − 8.57 (m, 2 H) | ++++ |
| 115 | 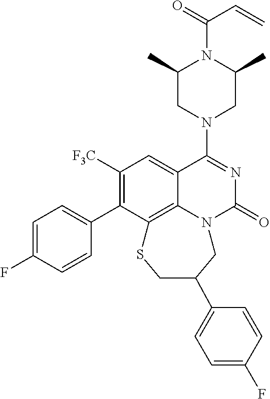 | 641.67 | ¹H NMR (400 MHz, CDCl₃) δ 8.11 (s, 1H), 7.35-7.29 (m, 3H), 7.25-7.16 (m, 3H), 7.04-6.99 (m, 2H), 6.67-6.60 (m, 1H), 6.42 (dd, J = 16.8 Hz, 2.0 Hz, 1H), 5.78 (dd, J = 10.8 Hz, 2.4 Hz, 1H), 4.78-4.63 (m, 4H), 4.21-4.15 (m, 2H), 3.75-3.63 (m, 2H), 3.42-3.33 (m, 2H), 3.06-3.03 (m, 1H), 1.60 (d, J = 6.8 Hz, 3H), 1.51 (d, J = 6.8 Hz, 3H). | ++ |
| 116 | 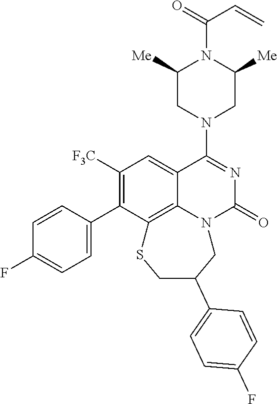 | 641.67 | ¹H NMR (400 MHz, CDCl₃) δ 8.16 (s, 1H), 7.40-7.35 (m, 3H), 7.27-7.19 (m, 3H), 7.06 (t, J = 11.6 Hz, 2H), 6.69 (dd, J = 22.0 Hz, 13.6 Hz, 1H), 6.47 (dd, J = 22.0 Hz, 2.4 Hz, 1H), 5.83 (dd, J = 13.6 Hz, 2.0 Hz, 1H), 4.87-4.63 (m, 4H), 4.26-4.18 (m, 2H), 3.80-3.67 (m, 2H), 3.47-3.36 (m, 2H), 3.11-3.06 (m, 1H), 1.70-1.62 (m, 3H), 1.57-1.52 (m, 3H). | +++ |
| 117 | 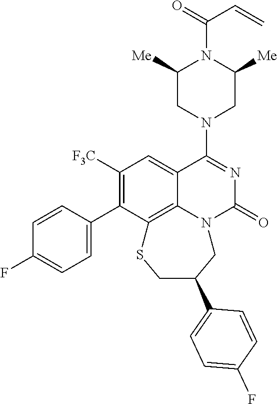 | 641.67 | ¹H NMR (400 MHz, CDCl₃) δ 8.16 (s, 1H), 7.40-7.35 (m, 3H), 7.27-7.20 (m, 3H), 7.06 (t, J = 11.6 Hz, 2H), 6.69 (dd, J = 22.4 Hz,13.6 Hz, 1H), 6.47 (dd, J = 22.0 Hz, 2.0 Hz, 1H), 5.83 (dd, J = 13.6 Hz, 2.0 Hz, 1H), 4.87-4.59 (m, 4H), 4.26-4.20 (m, 2H), 3.84-3.60 (m, 2H), 3.47-3.36 (m, 2H), 3.11-3.07 (m, 1H), 1.80-1.60 (m, 3H), 1.58-1.30 (m, 3H). | ++ |
| 118 | 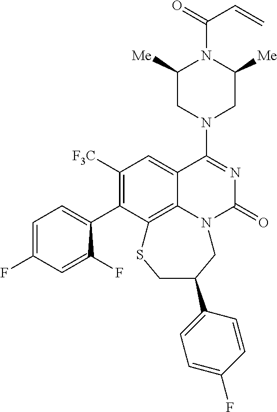 | 659.1 | ¹H NMR (400 MHz, CDCl₃) δ 8.14 (s, 1H), 7.35-7.32 (m, 2H), 7.20-7.14 (m, 1H), 7.05-6.96 (m, 4H), 6.67-6.60 (m, 1H), 6.42(dd, J = 16.4 Hz, 1.2 Hz, 1H), 5.78(dd, J = 10.4 Hz, 2.0 Hz, 1H), 4,78- 4.61 (m, 4H), 4.21- 4.16 (m, 2H), 3.76- 3.70 (m, 2H), 3.42- 3.33 (m, 2H), 3.08- 3.03 (m, 1H), 1.60 (d, J = 6.8 Hz, 3H), 1.52 (d, J = 6.8 Hz, 3H). | + |
| 119 | 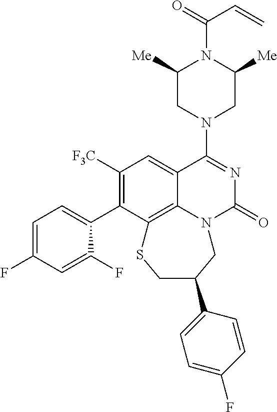 | 659.1 | ¹H NMR (400 MHz, CDCl₃) δ 8.12 (s, 1H), 7.32-7.21 (m, 3H), 7.04-6.94 (m, 4H), 6.64-6.60 (m, 1H), 6.42(d, J = 16.4 Hz, 1H), 5.78(d, J = 10.4 Hz, 1H), 4,76- 4.66 (m, 4H), 4.20- 4.15 (m, 2H), 3.75- 3.70 (m, 2H), 3.39- 3.33 (m, 2H), 3.09- 3.06 (m, 1H), 1.59- 1.38 (m, 6H). | ++ |
| 120 | 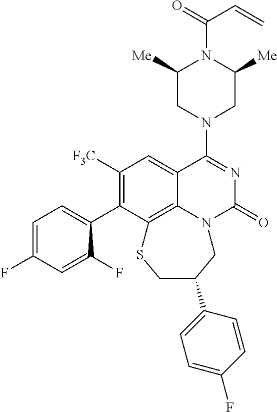 | 659.1 | ¹H NMR (400 MHz, CDCl₃) δ 8.12 (s, 1H), 7.35-7.31 (m, 2H), 7.23-7.20 (m, 1H), 7.06-6.98 (m, 3H), 6.95-6.92 (m, 1H), 6.67-6.60 (m, 1H), 6.39 (dd, J = 16.4 Hz, 2.0 Hz, 1H), 5.78 (dd, J = 10.4 Hz, 2.0 Hz, 1H), 4.78- 4.61 (m, 4H), 4.18 (t, J = 12.4 Hz, 2H), 3.80-3.69 (m, 2H), 3.40-3.33 (m, 2H), 3.09 (d, J = 13.6 Hz, 1H), 1.59 (d, J = 6.8 Hz, 3H), 1.51 (d, J = 6.8 Hz, 3H). | ++ |
| 121 | 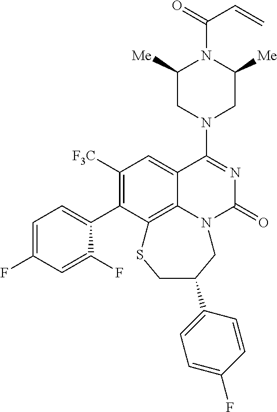 | 659.1 | ¹H NMR (400 MHz, CDCl₃) δ 8.14 (s, 1H), 7.40-7.30 (m, 2H), 7.20-7.14 (m, 1H), 7.08-6.96 (m, 4H), 6.67-6.60 (m, 1H), 6.42 (dd, J = 16.4 Hz, 2.0 Hz, 1H), 5.78 (dd, J = 10.4 Hz, 2.4 Hz, 1H), 4.81- 4.58 (m, 4H), 4.21- 4.16 (m, 2H), 3.85- 3.50 (m, 2H), 3.45- 3.30 (m, 2H), 3.20- 3.00 (m, 1H), 1.59 (d, J = 7.6 Hz, 3H), 1.52 (d, J = 6.4 Hz, 3H). | +++ |
| 122 | 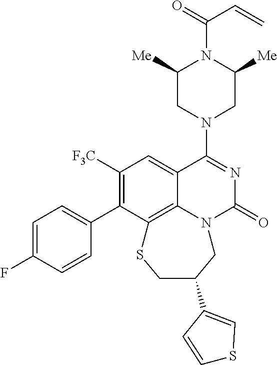 | 629.0 | ¹H NMR (400 MHz, CDCl₃) δ 8.10 (s, 1H), 7.31-7.29 (m, 1H), 7.24 (s, 1H), 7.20-7.15 (m, 4H), 7.10 (d, J = 4.8 Hz, 1H), 6.63 (dd, J = 16.4 Hz, 10.8 Hz, 1H), 6.42 (dd, J = 16.8 Hz, 1.6 Hz, 1H), 5.78 (dd, J = 10.4 Hz, 1.6 Hz, 1H), 4.87- 4.60 (m, 4H), 4.17 (t, J = 12.0 Hz, 2H), 3.89-3.85 (m, 1H), 3.62 (d, J = 10.4 Hz, 1H), 3.41-3.32 (m, 2H), 3.07 (d, J = 14.8 Hz, 1H), 1.60 (d, J = 7.2 Hz, 3H), 1.51 (d, J = 6.8 Hz, 3H). | +++ |
| 123 | 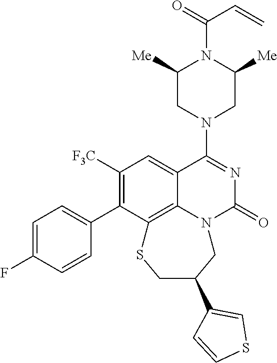 | 629.0 | ¹H NMR (400 MHz, CDCl₃) δ 8.10 (s, 1H), 7.31-7.29 (m, 1H), 7.24 (s, 1H), 7.20-7.15 (m, 4H), 7.10 (d, J = 4.0 Hz, 1H), 6.63 (dd, J = 16.0 Hz, 10.0 Hz, 1H), 6.42 (dd, J = 16.4 Hz, 1.6 Hz, 1H), 5.78 (dd, J = 10.4 Hz, 1.6 Hz, 1H), 4.85- 4.60 (m, 4H), 4.18 (t, J = 11.6 Hz, 2H), 3.88-3.84 (m, 1H), 3.62 (d, J = 8.8 Hz, 1H), 3.41-3.32 (m, 2H), 3.07 (d, J = 16.4 Hz, 1H), 1.60 (d, J = 7.2 Hz, 3H), 1.51 (d, J = 6.8 Hz, 3H). | ++ |
| 124 | 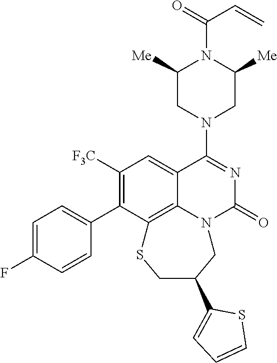 | 629.0 | ¹H NMR (400 MHz, CDCl₃) δ 8.09 (s, 1H), 7.28-7.27 (m, 1H), 7.21-7.15 (m, 4H), 7.00-6.99 (m, 1H), 6.97-6.95 (m, 1H), 6.63 (dd, J = 16.4 Hz, 10.4 Hz, 1H), 6.41 (dd, J = 16.8 Hz, 2.0 Hz, 1H), 5.78 (dd, J = 10.4 Hz, 2.0 Hz, 1H), 4.87- 4.84 (m, 1H), 4.76- 4.53 (m, 3H), 4.21- 4.15 (m, 2H), 4.11- 4.03 (m, 1H), 3.71- 3.61 (m, 1H), 3.41- 3.32 (m, 2H), 3.13- 3.02 (m, 1H), 1.60 (d, J = 7.2 Hz, 3H), 1.52 (d, J = 6.8 Hz, 3H). | ++ |
| 125 | 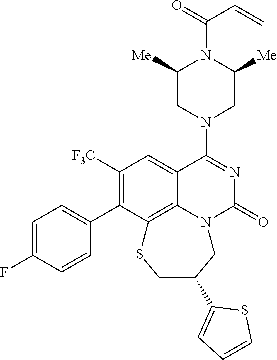 | 629.0 | ¹H NMR (400 MHz, CDCl₃) δ 8.09 (s, 1H), 7.31-7.29 (m, 1H), 7.20-7.17 (m, 4H), 7.00-6.99 (m, 1H), 6.97-6.95 (m, 1H), 6.63 (dd, J = 16.8 Hz, 10.4 Hz, 1H), 6.41 (dd, J = 16.8 Hz, 2.0 Hz, 1H), 5.78 (dd, J = 10.4 Hz, 1.6 Hz, 1H), 4.94- 4.83 (m, 1H), 4.78- 4.54 (m, 3H), 4.21- 4.15 (m, 2H), 4.11- 4.02 (m, 1H), 3.72- 3.60 (m, 1H), 3.41- 3.33 (m, 2H), 3.10- 3.03 (m, 1H), 1.60- 1.57 (m, 3H), 1.52 (d, J = 6.8 Hz, 3H). | +++ |
| 126 | 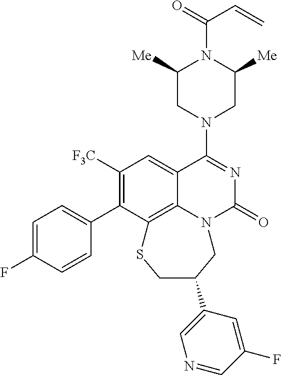 | 642.2 | ¹H-NMR (400 MHz, CDCl₃) δ 8.47 (s, 1H), 8.42 (s, 1H), 8.13 (S, 1H), 7.46 (m, 1H), 7.25-7.19 (m, 4 H), 6.61 (dd, J = 16 Hz, J = 6 Hz, 1H), 6.44 (d, J = 16 Hz, 1H), 5.80 (d, J = 6 Hz, 1H), 4.80 (m, 4H), 4.21 (m, 2H), 3.75 (m, 2H), 3.40 (m, 2H), 3.06 (m, 1H), 1.57 (m, 6H) | ++++ |

0.5-2.0 nM = ++++;
2.0 nM-20 nM = +++;
20 nM-250 nM = ++;
>250 nM = +

Although the foregoing embodiments have been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compound of Formula (I) or pharmaceutically acceptable salt thereof: 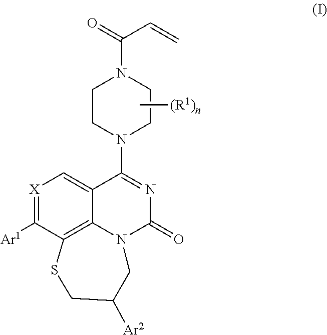
wherein:
each R¹ is independently methyl or cyanomethyl;
n is an integer from 0 to 2;
X is —CCF₃ or —C-halogen;
Ar¹ is aryl or heteroaryl, optionally substituted with one or more alkyl, halogen, hydroxy or amino; and
Ar² is a C-linked aryl or heteroaryl, optionally substituted with alkyl or halogen.

2. The compound of claim 1, wherein Ar¹ is phenyl optionally substituted with one or more halogens.

3. The compound of claim 1 or 2, wherein X is —CCl or —CCF₃.

4. The compound of any one of claims 1 to 3, wherein Ar¹ is a phenyl substituted with one or more halogens.

5. The compound of any one of claims 1 to 4, wherein n is 2 and each R¹ is methyl.

6. The compound of any one of claims 1 to 5, wherein Ar² is selected from: 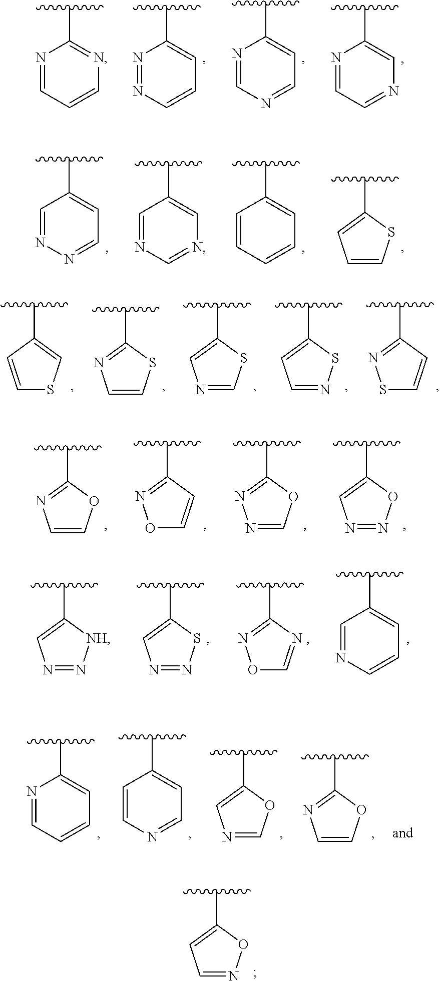
any of which are optionally substituted with halogen.

7. The compound of any one of claims 1 to 5, wherein the compounds is selected from: 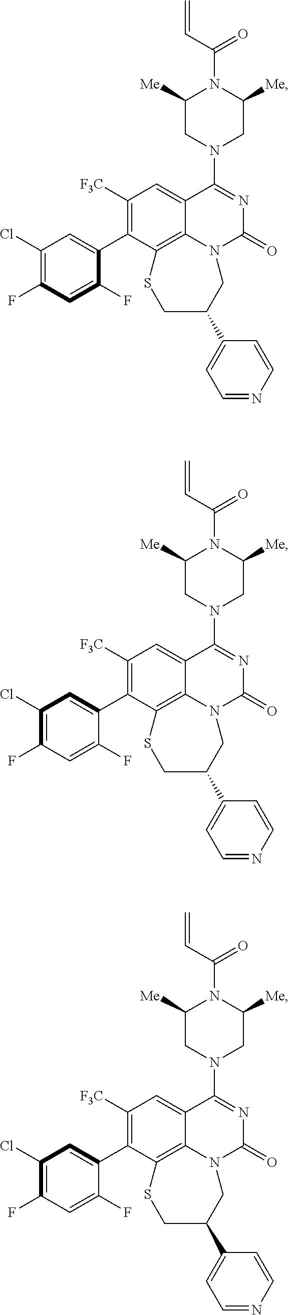 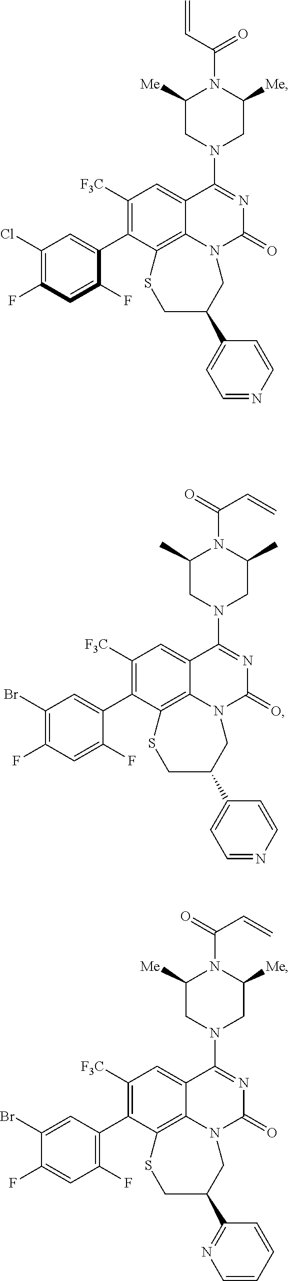 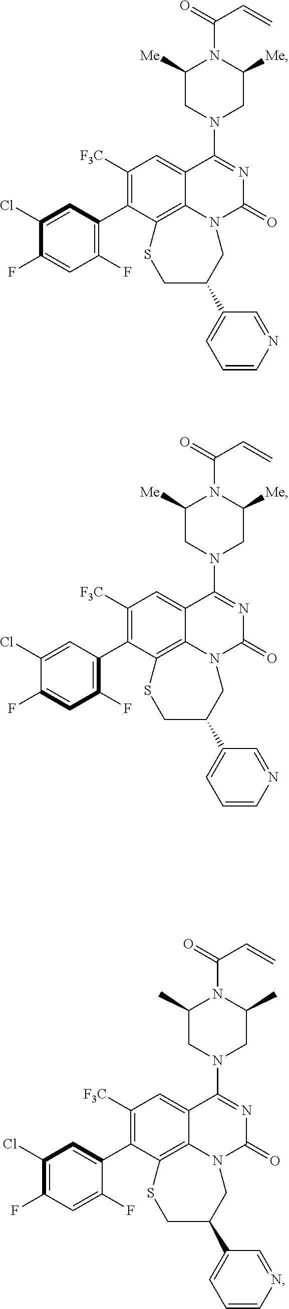 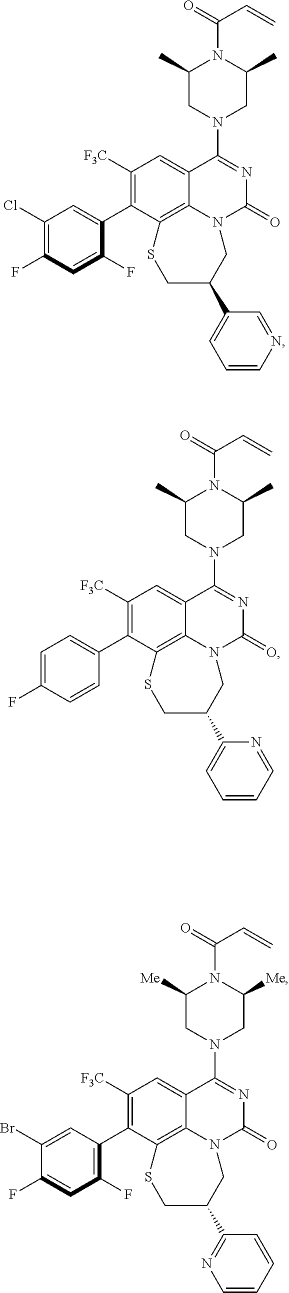 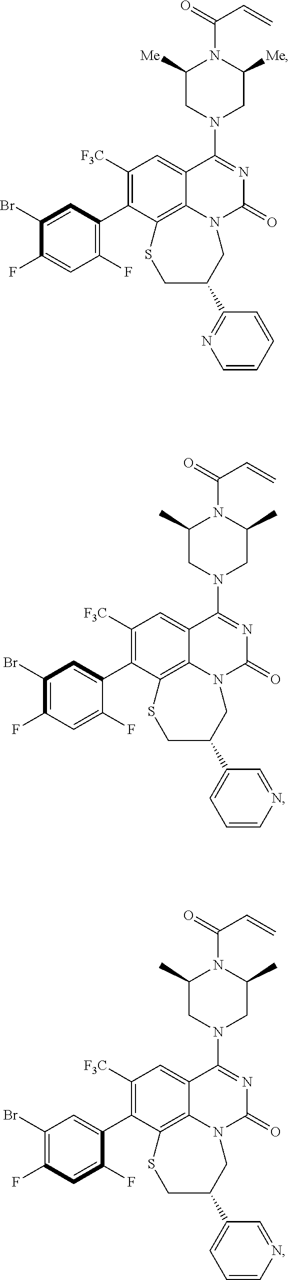 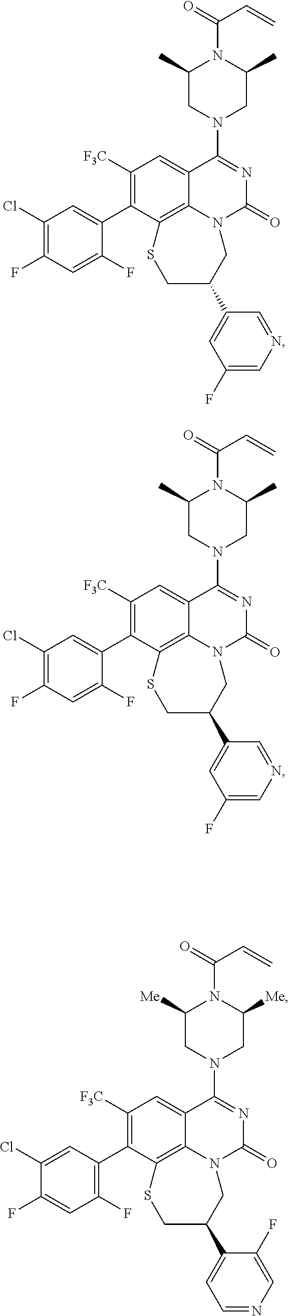 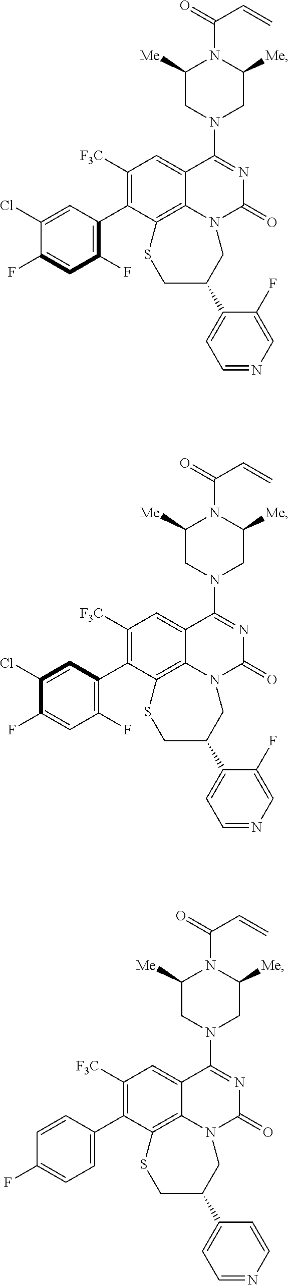 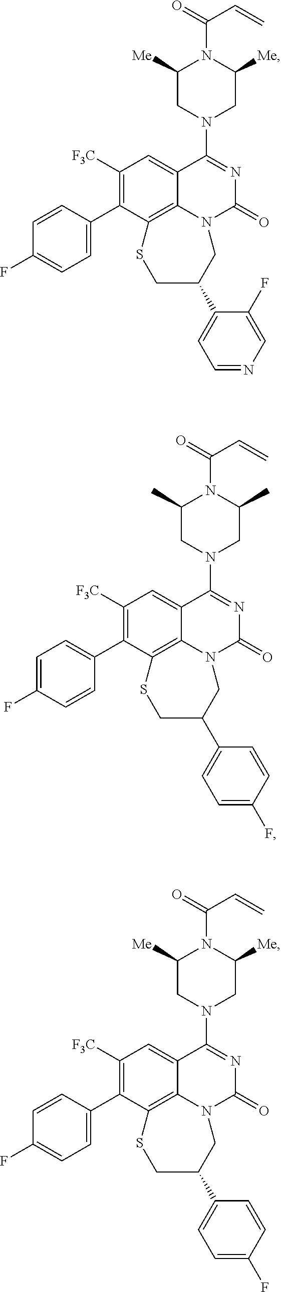 [SMILES: C=CC(=O)N1[C@H](C)CN(c2nc(=O)n3c4c(c(-c5ccc(F)cc5)c(C(F)(F)F)cc24)SC[C@@H](c2ccncc2F)C3)C[C@@H]1C ; C=CC(=O)N1[C@H](C)CN(c2nc(=O)n3c4c(c(-c5ccc(F)cc5)c(C(F)(F)F)cc24)SCC(c2ccc(F)cc2)C3)C[C@@H]1C ; C=CC(=O)N1[C@H](C)CN(c2nc(=O)n3c4c(c(-c5ccc(F)cc5)c(C(F)(F)F)cc24)SC[C@@H](c2ccc(F)cc2)C3)C[C@@H]1C] 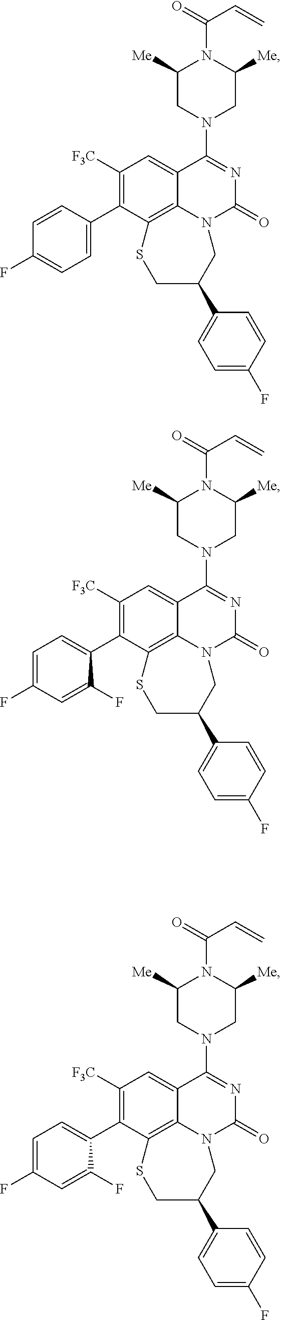 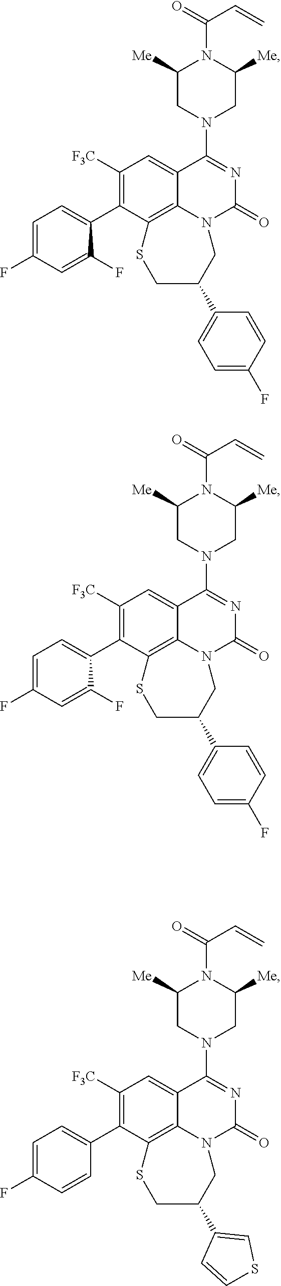 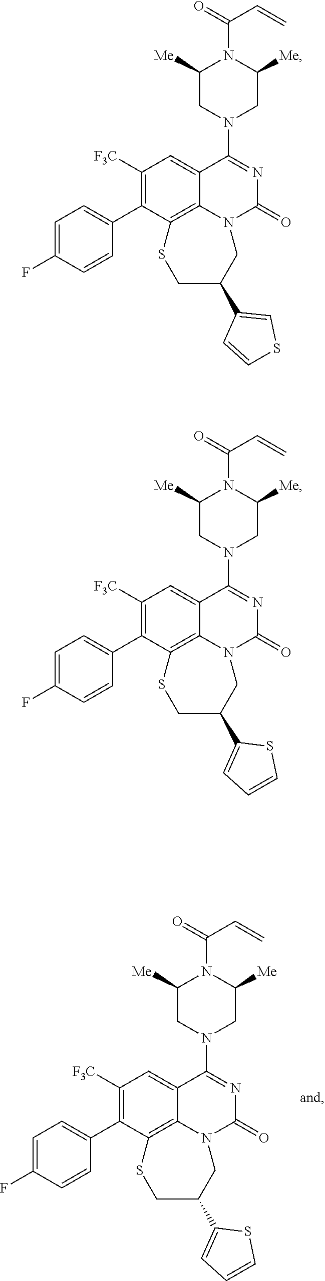 [SMILES: C=CC(=O)N1[C@H](C)CN(c2nc(=O)n3c4c(c(-c5ccc(F)cc5)c(C(F)(F)F)cc24)SC[C@H](c2ccsc2)C3)C[C@@H]1C ; C=CC(=O)N1[C@H](C)CN(c2nc(=O)n3c4c(c(-c5ccc(F)cc5)c(C(F)(F)F)cc24)SC[C@H](c2cccs2)C3)C[C@@H]1C ; C=CC(=O)N1[C@H](C)CN(c2nc(=O)n3c4c(c(-c5ccc(F)cc5)c(C(F)(F)F)cc24)SC[C@@H](c2cccs2)C3)C[C@@H]1C] 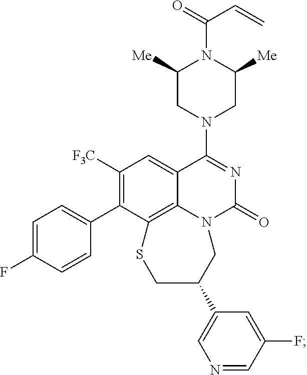 [SMILES: C=CC(=O)N1[C@H](C)CN(c2nc(=O)n3c4c(c(-c5ccc(F)cc5)c(C(F)(F)F)cc24)SC[C@@H](c2cncc(F)c2)C3)C[C@@H]1C]
or a pharmaceutically acceptable salt thereof.

8. A pharmaceutical composition comprising a compound, or pharmaceutically acceptable salt thereof, of any one of claims 1 to 7 in a pharmaceutically acceptable carrier.

9. A method of treating a subject with cancer comprising administering to the subject a compound, or pharmaceutically acceptable salt thereof, according to any one of claims 1 to 7, or a pharmaceutical composition thereof.

10. The method of claim 9, wherein the cancer comprises a K-Ras G12 mutation.

11. The method of claim 10, wherein the G12 mutation is G12C.

12. The method of any one of claims 9 to 11, wherein the cancer is one or more of pancreatic, lung, and colorectal cancer.

13. The method of claim 12, wherein the pancreatic cancer is pancreatic ductal adenocarcinoma (PDAC).

14. The method of any one of claims 9 to 13, wherein the cancer is a CNS cancer.

15. The method of claim 14, wherein the CNS cancer is a primary cancer.

16. The method of claim 15, wherein the primary cancer comprises one or more of a glioma, meningioma, medulloblastoma, ganblioglioma, schwannoma, and craniopharyngioma.

17. The method of claim 16, wherein the glioma comprises one or more of an astrocytoma, a glioblastoma, an oligodendroglioma, and a ependymoma.

18. The method of claim 14, wherein the CNS cancer comprises a metastatic or secondary cancer.

19. The method of claim 18, wherein the CNS cancer comprises a cancer metastasized from one or more of melanoma, breast cancer, colon cancer, kidney cancer, nasopharyngeal cancer, leukemia, lymphoma, myeloma, and other of unknown primary site.

20. Use of a compound, or pharmaceutically acceptable salt thereof, according to any one of claims 1 to 7 in the treatment of cancer.

21. The use of claim 20, wherein the cancer is one or more of pancreatic, lung and colorectal cancer.

22. The use of claim 20, wherein the cancer is a CNS cancer.

23. The use of claim 22, wherein the CNS cancer comprises a primary cancer.

24. The use of claim 23, wherein the primary cancer comprises one or more of a glioma, meningioma, medulloblastoma, ganblioglioma, schwannoma, and craniopharyngioma.

25. The use of claim 24, wherein the glioma comprises one or more of an astrocytoma, a glioblastoma, an oligodendroglioma, and an ependymoma.

26. The use of claim 22, wherein the CNS cancer comprises a metastatic or secondary cancer.

27. The use of claim 26, wherein the CNS cancer comprises a cancer metastasized from one or more of melanoma, breast cancer, colon cancer, kidney cancer, nasopharyngeal cancer, leukemia, lymphoma, myeloma, and other of unknown primary site.

28. The use of claim 22, wherein the CNS cancer comprises a RAS associated cancer.

29. A method of preventing or reducing the spread of cancer via CNS pathways comprising administering to the subject a compound according to any one of claims 1 to 7, a pharmaceutically acceptable salt, or a pharmaceutical composition thereof.

30. Use of a compound, or pharmaceutically acceptable salt thereof, of any one of claims 1 to 7 or pharmaceutical composition thereof in the manufacture of a medicament for preventing or reducing the spread of cancer via CNS pathways.


SEQUENCE LISTING (3 sequences)

SEQ ID NO: 1 - protein, 169 aa, synthetic construct
  REGION 1..169: note = Synthetic Polypeptide
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 2 - protein, 188 aa, synthetic construct [sequence omitted]

SEQ ID NO: 3 - protein, 188 aa, synthetic construct [sequence omitted]